(19) United States
(12) Patent Application Publication
(10) Pub. No.: US 2019/0203240 A1
(43) Pub. Date: Jul. 4, 2019

(54) METHODS FOR THE PRODUCTION OF RHAMNOSYLATED FLAVONOIDS

(71) Applicant: Universitaet Hamburg, Hamburg (DE)

(72) Inventors: Ulrich Rabausch, Hamburg (DE); Henning Rosenfeld, Hamburg (DE); Nele Ilmberger, Hamburg (DE); Tanja Plambeck, Norderstedt (DE); Constantin Ruprecht, Hamburg (DE); Friederike Boenisch, Hamburg (DE)

(21) Appl. No.: 16/069,302

(22) PCT Filed: Jan. 13, 2017
(86) PCT No.: PCT/EP2017/050691

§ 371 Date: Jul. 11, 2018

(30) Foreign Application Priority Data
Jan. 15, 2016 (DE) 16151612.5

(51) Int. Cl. C12P 19/18; C12P 19/60

(52) U.S. Cl. CPC C12P 19/18; C12Y 204/01; C12P 19/60

(57) ABSTRACT

A method for the production of rhamnosylated flavonoids comprising the steps of contacting/incubating a glycosyl transferase with a flavonoid and obtaining a rhamnosylated flavonoid. In addition, glycosyl transferases suitable for use in such methods and kits comprising said glycosyl transferases.

The present invention relates to methods for the production of rhamnosylated flavonoids comprising the steps of contacting/incubating a glycosyl transferase with a flavonoid and obtaining a rhamnosylated flavonoid. In addition, the invention relates to glycosyl transferases suitable for use in such methods and kits comprising said glycosyl transferases.

Flavonoids are a class of polyphenol compounds which are commonly found in a large variety of plants. Flavonoids comprise a subclass of compounds such as anthoxanthins, flavanones, flavanonols, flavans and anthocyanidins. Flavonoids are known to possess a multitude of beneficial properties which make these compounds suitable for use as antioxidants, anti-inflammatory agents, anti-cancer agents, antibacterials, antivirals, antifungals, antiallergenes, and agents for preventing or treating cardiovascular diseases. Furthermore, some flavonoids have been reported to be useful as flavor enhancing or modulating agents.

Due to this wide variety of possible applications, flavonoids are compounds of high importance as ingredients in cosmetics, food, drinks, nutritional and dietary supplements, pharmaceuticals and animal feed. However, use of these compounds has often been limited due to the low water solubility, low stability and limited availability. A further factor which has severely limited use of these compounds is the fact that only a few flavonoids occur in significant amounts in nature while the abundance of other flavonoids is nearly negligible. As a result, many flavonoids and their derivatives are not available in amounts necessary for large-scale industrial use.

Glycosylation is one of the most abundant modifications of flavonoids, which has been reported to significantly modulate the properties of these compounds. For example, glycosylation may lead to higher solubility and increased stability, such as higher stability against radiation or temperature. Furthermore, glycosylation may modulate pharmacological activity and bioavailability of these compounds.

Glycosylated derivatives of flavonoids occur in nature as O-glycosides or C-glycosides, while the latter are much less abundant. Such derivatives may be formed by the action of glycosyl transferases (GTs) starting from the corresponding aglycones.

Examples of naturally occurring O-glycosides are quercetin-3-O-β-D-glucoside (Isoquercitrin) and genistein-7-O-β-glucoside (Genistin).

However, flavonoids constitute the biggest class of polyphenols in nature (Ververidis (2007) Biotech. J. 2(10):1214-1234). The high variety of flavonoids originates from addition of various functional groups to the ring structure. Herein, glycosylation is the most abundant form and the diversity of sugar moieties even more leads to a plethora of glycones.

But in nature only some flavonoid glycones prevail. As described above, among these are the 3-O-β-D-glucosides, e.g. isoquercitrin, the flavonoid-7-β-D-glucosides, e.g. genistin, and the 3- and 7-rhamnoglucosides, e.g. rutin and naringin. Generally, glucosides are the most frequent glycosidic forms with 3- and 7-O-β-D-glucosides dominating. In contrast, glycosides concerning other sugar moieties, e.g. rhamnose, and other glycosylation positions than C3 and C7 rarely occur and are only present in scarce quantities in specific plant organs. This prevents any industrial uses of such compounds. For example, De Bruyn (2015) Microb Cell Fact 14:138 describes methods for producing rhamnosylated flavonoids at the 3-O position. Also, 3-O rhamnosylated versions of naringenin and quercetin are described by Ohashi (2016) Appl Microbiol Biotechnol 100:687-696. Metabolic engineering of the 3-O rhamnoside pathway in E. coli with kaempferol as an example is described by Yang (2014) J Ind Microbiol Biotech 41:1311-18. Finally, the in vitro production of 3-O rhamnosylated quercetin and kaempferol is described by Jones (2003) J Biol Chem 278:43910-18. None of these documents describes or suggests the production of 5-O rhamnosylated flavonoids.

In fact, very few examples of 5-O rhamnosylated flavonoids are known in the art. The few examples are quercetin-5-O-β-D-glucoside, luteolin-5-O-glucoside, and chrysin-5-O-β-D-xyloside (Hedin (1990) J Agric Food Chem 38(8):1755-7; Hirayama (2008) Photochemistry 69(5):1141-1149; Jung (2012) Food Chem Toxicol 50(6):2171-2179; Chauhan (1984) Phytochemistry 23(10):2404-2405). Up to now, only four flavonoid-5-O-rhamnosides were described. Taxifolin-3,5-di-O-α-L-rhamnoside was extracted from the Indian plant Cordia obliqua which also contains low amounts of Hesperetin-7-O-α-L-rhamnoside (Chauhan (1978) Phytochemistry 17:334; Srivastava (1979) Phytochemistry 18:2058-2059). Eriodictyol-5-rhamnoside was isolated from Cleome viscosa (Srivastava (1979) Indian J Chem Sect B 18:86-87). Another flavanone, Naringenin-5-O-α-L-rhamnoside (N5R) was isolated from Himalayan cherry (Prunus cerasoides) seeds (Shrivastava (1982) Indian J Chem Sect B 21 (6):406-407). Extraction from 2 kg of air dried powdered seeds resulted in 800 mg N5R. The absolute rare occurrence inhibits the commercial use also of other flavanone rhamnosides like naringenin-4′-O-α-L-rhamnoside that was isolated from the stem of a tropical Fabaceae plant (Yadava (1997) J Indian Chem Soc 74(5):426-427).

WO 2014/191524 relates to enzymes catalyzing the glycosylation of polyphenols, in particular flavonoids, benzoic acid derivatives, stilbenoids, chalconoids, chromones, and coumarin derivatives. In addition, WO 2014/191524 discloses methods for preparing a glycoside of polyphenols. However, glycosylation is limited to C3, C3′, C4′ and C7 of polyphenols. Moreover, the disclosure is silent with regard to the possibility of rhamnosylating polyphenols.

Accordingly, there is an urgent need for reliable methods for the large-scale production of 5-O rhamnosylated flavonoids to allow commercial use.

Thus, the technical problem underlying the present invention is the provision of reliable means and methods for efficient rhamnosylation of flavonoids at C5, corresponding to the R³ position of Formula I.

The technical problem is solved by provision of the embodiments characterized in the claims.

Accordingly, the present invention relates to methods for the production of rhamnosylated flavonoids comprising contacting/incubating a glycosyl transferase with a flavonoid and obtaining a rhamnosylated flavonoid. In this regard, it has been surprisingly and unexpectedly found that glycosyl transferases are able to rhamnosylate flavonoids at the C5-OH, i.e. R³ position, in particular where the flavonoid is represented by the following formula (I):

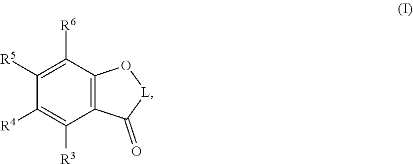

In contrast to what could have been expected based on the prior art, glycosyl transferases are able to rhamnosylate compounds of formula I at the R³ position, corresponding to C5 of polyphenols as described in WO 2014/191524. Accordingly, as illustrated in the appended Examples, the methods of the present invention allow the production of 5-O rhamnosides, in particular at large-scale to allow the commercial use of the produced 5-O rhamnosides. In this regard, it was surprisingly found that most efficient production of rhamnosylated flavonoids can be observed in experiments using concentrations of the reactant, i.e. the flavonoid, above its solubility in aqueous solutions. That is, the present invention relates to methods for the production of rhamnosylated flavonoids comprising contacting/incubating a glycosyl transferase with a flavonoid, wherein the flavonoid is contacted/incubated with said glycosyl transferase at a final concentration above its solubility in aqueous solutions, preferably above about 200 μM, more preferably above about 500 μM, and even more preferably above about 1 mM, and subsequently obtaining a rhamnosylated flavonoid. The skilled person will appreciate that the solubility varies depending on the flavonoid used as educt in the methods of the present invention. Thus, the above values can be altered depending on the used flavonoid.

In the methods of the present invention, a glycosyl transferase is used for efficient production of 5-O rhamnosylated flavonoids. In principle, any glycosyltransferase may be used, as is evidenced by the appended Examples; see e.g. Example A3, in particular Tables A7 and A8. However, it is preferred that a glycosyl transferase belonging to family GT1 is used. In this regard, the glycosyl transferases GTC, GTD, GTF, and GTS belong to the glycosyltransferase family GT1 (EC 2.4.1.x) (Coutinho (2003) J Mol Biol 328(2):307-317). This family comprises enzymes that mediate sugar transfer to small lipophilic acceptors. Family GT1 members uniquely possess a GT-B fold. They catalyze an inverting reaction mechanism concerning the glycosidic linkage in the sugar donor and the formed one in the acceptor conjugate, creating natural β-D- or α-L-glycosides.

Within the GT-B fold the enzymes form two major domains, one N-terminal and a C-terminal, with a linker region in between. Generally, the N-terminus constitutes the AA-residues responsible for acceptor binding and the residues determining donor binding are mainly located in the C-terminus. In family GT1 the C-terminus contains a highly conserved motif possessing the AA residues that take part in nucleoside-diphosphate (NDP)-sugar binding. This motif was also termed the plant secondary product glycosyltransferase (PSPG) box (Hughes (1994) Mit DNA 5(1):41-49.

Flavonoid-GTs belong to family GT1. Due to the natural biosynthesis of flavonoids in plants most of the enzymes are also known from plants. However, several enzymes from the other eukaryotic kingdoms fungi and animals and also from the domain of bacteria are described. In eucarya, sugar donors of GT1 enzymes are generally uridinyl-diphosphate (UDP)-activated. Of these so called UGTs or UDPGTs, most enzymes transfer glucose residues from UDP-glucose to the flavonoid acceptors. Other biological relevant sugars from UDP-galactose, -rhamnose, -xylose, -arabinose, and -glucuronic acid are less often transferred.

Also several bacterial GT1s were discovered that are able to glycosylate also flavonoid acceptors. These enzymes all belong to the GT1 subfamily of antibiotic macrolide GTs (MGT). In bacteria GT1 enzymes use UDP-glucose or galactose but also deoxythymidinyl-diphosphate (dfDP)-activated sugars as donor substrates. However, all the bacterial flavonoid active GT1 enzymes have UDP-glucose as the native donor. There is only one known exception with the metagenome derived enzyme GtfC that was the first bacterial GT1 reported to transfer rhamnose to flavonoids (Rabausch (2013) Appl Environ Microbiol 79(15):4551-4563). However, until the present invention was made, it was established that this activity is limited to C3-OH or the C7-OH groups of flavonoids. Transfer to the C3′-OH and the C4′-OH of the flavonoid C-ring was already less commonly observed. Other positions are rarely glycosylated, if at all. Specifically, there are only few examples concerning the glycosylation of the C5-OH group, which is based on the fact that this group is sterically protected if a keto group at C4 is present. Therefore, the only examples relate to anthocyanidins (Janvary (2009) J Agric Food Chem 57(9):3512-3518; Lorenc-Kukala (2005) J Agric Food Chem 53(2):272-281; Tohge (2005) The Plant J 42(2):218-235). This class of flavonoids lacks the C4 keto group which facilitates nucleophilic attack. The C5-OH group of (iso)flavones and (iso)flavanones is protected through hydrogen bridges with the neighbored carbonyl group at C4. This was thought to even hinder chemical glycosylation approaches at C5 of these classes.

Today, there are only three GT1 enzymes characterized that create 5-O-β-D-glucosides of flavones. One is UGT71G1 from Medicago truncatula which was proven to be not regio-selective and showed a slight side activity in glucosylation of C5-OH on quercetin (He (2006) JBC 281(45):34441-7. An exceptional UGT was identified in the silkworm Bombyx mori capable of specifically forming quercetin-5-O-β-D-glucoside (Daimon (2010) PNAS 107(25):11471-11476; Xu (2013) Mol Biol Rep 40(5):3631-3639) Finally, a mutated variant of MGT from Streptomyces lividans presented low activity at C5-OH of 5-hydroxyflavone after single AA exchange (Xie (2013) Biochemistry (Mosc) 78(5):536-541). However, the wild type MGT did not possess this ability nor did other MGTs.

Flavanol-5-O-α-D-glucosides were synthesized through transglucosylation activity of hydrolases, i.e. α-amylases (EC 3.2.1.x) (Noguchi (2008) J Agric Food Chem 56(24):12016-12024; Shimoda (2010) Nutrients 2(2):171-180). However, the flavanols also lack the C4=O-group and the enzymes create a “non-natural” α-D-glucosidic linkage.

It is noteworthy that all so far known 5-O-GTs mediated only glucosylation. The prior art is entirely silent with regard to rhamnosylation of flavonoids, much less using the method of the present invention.

Thus, GTC from Elbe river sediment metagenome, GTD from Dyadobacter fermentans, GTF from Fibrosoma limi, and GTS from Segetibacter koreensis and chimeras 1, 3, and 4 are the first experimentally proved flavonoid-5-O-rhamnosyltransferases (FRTs). This is evidenced by the appended Examples. In particular, Example A3 provides results for all chimeras in Tables A7 and A8. Further production examples are shown in the further Examples, in particular using GTC. Furthermore, related enzymes from, Flavihumibacter solisilvae, Cesiribacter andamanensis, Niabella aurantiaca, Spirosoma radiotolerans, Fibrella aestuarina, Flavisolibacter sp. LCS9 and Aquimarina macrocephali, present the same functionality as they share important amino acid sequence features. In contrast to all other GT1 enzymes that use NDP-sugars FRTs possess several unique amino acid patterns.

Accordingly, the present invention relates to a method for the production of 5-O, i.e. R³ in formula I, rhamnosylated flavonoids using a glycosyl transferase comprising said conserved amino acids. These conserved amino acid sequences, which were surprisingly and unexpectedly identified by the present inventors, comprise the following motifs (all amino acid positions are given with respect to the wild-type GTC amino acid sequence): (1) strictly conserved amino acids Asp (D³⁰) and aromatic Phe (F³³) in the motif ²¹K/R ILFAXXPXDGHF N/S PLTX L/I A⁴⁰ both located around His³², i.e. the active site residue of GT1 enzymes, wherein the amino acid at position 30 is preferably a polar amino acid; (2) the motif ⁴⁷GXDVRW Y/F⁵³ comprising the loop before Nβ2 and strand Nβ2; (3) strictly conserved amino acid Arg (R⁸⁸) of motif ⁸⁵F/Y/L P E/D R⁸⁸ where Pro⁸⁶ and Glu⁸⁷ are reported for substrate binding in GT1 enzymes and neighboring Arg (R⁸⁸) is unique to Rhamnosyl-GTs; (4) strictly conserved amino acids Phe (F¹⁰⁰), Asp (D¹⁰¹), Phe (F¹⁰⁶), Arg (R¹⁰⁹) and Asp (D¹¹⁶) of the motif ¹⁰⁰FDXXXXFXXRXXE Y/F XXD¹¹⁶ forming the long N-terminal helix Nα3, wherein the amino acids at positions 103 and 108 preferably are non-polar amino acids; (5) the motif ¹²⁴F/W PFXXXXX D/E XXFXXXXF¹⁴⁰ comprising the loop before Nβ4, strand Nβ4, and the loop to the downstream N-α-helix, wherein amino acids at positions 128 to 130 are preferably non-polar amino acids; (6) the motif ¹⁵⁶PLXEXXXXL P/A PXGXGXXPXXXXXG K/R¹⁸⁰ comprising conserved amino acid Gly (G¹⁷⁰); (7) the motif ²³⁰LQXGXXGFEYXR²⁴¹ before the linker region of the N-terminal domain with the C-terminal domain; (8) the motif ²⁸¹TQGTXE K/R XXXKXXXPTLEAF R/K³⁰¹ comprising the loop before Cα1 and helix Cα1 and strictly conserved amino acids Thr (T²⁸⁴) and Glu (G²⁸⁶) where Thr is involved in substrate binding and wherein the amino acid at position 285 preferably is a non-polar amino acid and amino acids at positions 292 to 294 preferably are non-polar amino acids; (9) the motif ³⁰⁶LVXXTTGG³¹³ forming strand Cβ2, wherein amino acids at positions 308 and 309 preferably are non-polar amino acids; and (10) the motif ³³⁰I E/D DFIPFXX V/I MPXXDV Y/F I/V T/S NGG Y/F GGV M/L LXIX N/H XLPXVXAGXH EGKNE³⁷⁶ comprising conserved acidic amino acids Glu/Asp (E/D³³¹), Asp (D³³²), conserved aromatic amino acid Phe (F³³⁶) instead of Gln (Q) in other GT1 enzymes at start of helix Cα2, strictly conserved amino acid Asn (N³⁴⁹) involved in substrate binding, and strictly conserved amino acid Gly (G³⁶⁹) instead of Pro (P) in other GT1 enzymes, wherein the motif forms the conserved donor binding region of GT1 enzymes, wherein the amino acids at positions 367 and 372 preferably are non-polar amino acids and where the ³⁷¹HEGKNE³⁷⁶ motif is absolutely unique to the 5-O-FRTs, as GT 1 enzymes usually show a D/E Q/N/K/R motif responsible for hexose sugar binding and catalytic activity.

The following alignment of said 5-O-FRTs illustrates the homologous AAs positions and shows consensus SEQ ID NO:1.

| | |
|---|---|
| | ....\|....\| ....\|....\|.... \|....\| ....\|....\| |
| | 5 15 25 35 |
| GTC | ---------M SNLFSSQTNL ASVKPLKGRK ILFANFPADG |
| GTD | ---------M TKYKN----- ----ELTGKR ILFGTVPGDG |
| GTF | ---------M TTK------- ---------K ILFATMPMDG |
| GTS | ---------- MKYIS----- ---SIQPGTK ILFANFPADG |
| GT from S. radiotolerans | --------MI TPQ------- ---------R ILFATMPMDG |
| GT from N. aurantiaca | --------MY TKTANTTNAA APLHGGEKKK ILFANIPADG |
| GT from F. solisilvae | ---------M NHKHS----- --------RK ILMANVPADG |
| GT from F. aestuarina | ---------M NPQ------- ---------R ILFATMPFDG |
| GT from C. andamanensis | METSQKGGTQ SPKPF----- --------RR ILFANCPADG |
| GT from A. macrocephali | ---------M TRMSQ----- --------KK ILFACIPADG |
| GT from F. sp. LCS9 | MNNTLSTVID HTIAS----- ---QIKPGTK ILFATFPADG |
| Chimera 1 | ---------M TKYKN----- ----ELTGKR ILFGTVPGDG |
| Chimera 3 | ---------M TKYKN----- ----ELTGKR ILFGTVPGDG |
| Chimera 4 | ---------M TKYKN----- ----ELTGKR ILFGTVPGDG |
| SEQ ID NO. 1 | ---------- ---------- ---------K ILFAXXPXDG |
| alternate aa SEQ ID NO. 1 | R |
| | ....\|....\| ....\|....\|.... \|....\| ....\|....\| |
| | 45 55 65 75 |
| GTC | HFNPLTGLAV HLQWLGCDVR WYTSNKYADK LRRLNIPHFP |
| GTD | HFNPLTGLAK YLQELGCDVR WYASDVFKCK LEKLSIPHYG |
| GTF | HFNPLTGLAV HLHNQGHDVR WYVGGHYGAK VKKLGLIHYP |
| GTS | HFNPLTGLAV HLKNIGCDVR WYTSKTYAEK IARLDIPFYG |
| GT from S. radiotolerans | HFSPLTGLAV HLSNLGHDVR WYVGGEYGEK VRKLKLHHYP |
| GT from N. aurantiaca | HFNPLTGLAV RLKKAGHDVR WYTGASYAPR IEQLGIPFYL |
| GT from F. solisilvae | HFNPLTGIAV HLKQQGYDVR WYGSDVYSKK AAKLGIPYFP |
| GT from F. aestuarina | HFSPLTNLAV HLSQLGHDVR WFVGGHYGQK VTQLGLHHYP |
| GT from C. andamanensis | HFNPLIPLAE FLKQQGHDVR WYSSRLYADK ISRMGIPHYP |
| GT from A. macrocephali | HFNPMTAIAI HLKTKGYDVR WYTGEGYKNT LHRIGIPYLP |
| GT from F. sp. LCS9 | HFNPLTGLAM HLKQIGCDVR WYTAKKYANK LQQLDIPHYD |
| Chimera 1 | HFNPLTGLAK YLQELGCDVR WYASDVFKCK LEKLSIPHYG |
| Chimera 3 | HFNPLTGLAK YLQELGCDVR WYASDVFKCK LEKLSIPHYG |
| Chimera 4 | HFNPLTGLAK YLQELGCDVR WYASDVFKCK LEKLSIPHYG |
| SEQ ID NO. 1 | HFNPLTXLA- -----GXDVR WY-------- ---------- |
| alternate aa SEQ ID NO. 1 | S I F |
| | ....\|....\| ....\|....\|.... \|....\| ....\|....\| |
| | 85 95 105 115 |
| GTC | FRKAMDIA-- -DLENMFPER DAIKGQVAKL KFDIINAFIL |
| GTD | FKKAWDVNG- VNVNEILPER QKLTDPAEKL SFDLIHIFGN |
| GTF | YHKAQVINQ- ENLDEVFPER QKIKGTVPRL RFDLNNVFLL |
| GTS | LQRAVDVSAH AEINDVFPER KKYKGQVSKL KFDMINAFIL |
| GT from S. radiotolerans | FVNARTINQ- ENLEREFPER AALKGSIARL RFDIKQVFLL |
| GT from N. aurantiaca | FNKAKEVTV- HNIDEVFPER KTIRNHVKKV IFDICTYFIE |
| GT from F. solisilvae | FSKALEVNS- ENAEEVFPER KRINSKIGKL NFDLQNFFVR |
| GT from F. aestuarina | YVKTRTVNQ- ENLDQLFPER ATIKGAIARI RFDLGQIFLL |
| GT from C. andamanensis | FKKALEFDT- HDWEGSFPER SKHKSQVGKL RFDLEHVFIR |
| GT from A. macrocephali | FQNAQELKI- EEIDKMYPDR KMLKG-IAHI KFDIINLFIN |
| GT from F. sp. LCS9 | LVRALDFAS- GEPDEIFPER KQHKSQLAKL KFDIINVFIK |
| Chimera 1 | FKKAWDVNG- VNVNEILPER QKLTDPAEKL SFDLIHIFGN |
| Chimera 3 | FKKAWDVNG- VNVNEILPER QKLTDPAEKL SFDLIHIFGN |
| Chimera 4 | FKKAWDVNG- VNVNEILPER QKLTDPAEKL SFDLIHIFGN |
| SEQ ID NO. 1 | ---------- ------FPER ---------- -FDXXXXFXX |
| alternate aa SEQ ID NO. 1 | Y D |
| alternate aa SEQ ID NO. 1 | L |
| | ....\|....\| ....\|....\|.... \|....\| ....\|....\| |
| | 125 135 145 155 |
| GTC | RGPEYYVDLQ EIHKSFPFDV MVADCAFTGI PFVTDKMDIP |
| GTD | RAPEYYEDIL EIHESFPFDV FIADSCFSAI PLVSKLMSIP |
| GTF | RAPEFITDVT AIHKSFPFDL LICDTMFSAA PMLRHILNVP |
| GTS | RSTEYYEDIL EIYEEFPFQL MIADITFGAI PFVEEKMNIP |
| GT from S. radiotolerans | RAPEFVEDMK DIYQTWPFTL VVHDVAFIGG SFIKQLLPVK |
| GT from N. aurantiaca | RGTEFYED1K DINKSFDFDV LICDSAFTGM SFVKEKLNKH |
| GT from F. solisilvae | RAPEYYADLI DIHREFPFDL LIADCMFTAI PFVKELMQIP |
| GT from F. aestuarina | RVPEQIDDLR AIYDEWPFDL IVQDLGFVGG TFLRELLPVK |
| GT from C. andamanensis | RGPEYFEDIR DLHQEFPFDV LVAEISFTGI AFIRHLMHKP |
| GT from A. macrocephali | RMKGYYEDIA EIHQVFPFDI LVCDNTFPGS -IVKKKLNIP |
| GT from F. sp. LCS9 | RGPEFYDDIK EIHQTFPFEV MIADVAFTGT PMVKEKMNIP |
| Chimera 1 | RAPEYYEDIL EIHESFPFDV FIADSCFSAI PLVSKLMSIP |
| Chimera 3 | RAPEYYEDIL EIHESFPFDV FIADSCFSAI PLVSKLMSIP |
| Chimera 4 | RAPEYYEDIL EIHESFPFDV FIADSCFSAI PLVSKLMSIP |
| SEQ ID NO. 1 | RXXEYXXD-- -----FPFXX XXXDXXFXXX XF-------- |
| alternate aa SEQ ID NO. 1 | F W E |
| | ....\|....\| ....\|....\|.... \|....\| ....\|....\| |
| | 165 175 185 195 |
| GTC | VVSVGVFPLT ETSKDLPPAG LGITPSFSLP GKFKQSILRS |
| GTD | VVAVGVIPLA EESVDLAPYG TGLPPAATEE QRAMYFGMKD |
| GTF | VAAVGIVPLS ETSKELPPAG LGMEPATGFF GRLKQDFLRF |
| GTS | VISISVVPLP ETSKDLAPSG LGITPSYSFF GKIKQSFLRF |
| GT from S. radiotolerans | TVAVGVVPLT ESDDYLPPSG LGRQPMRGIA GRWIQHLMRY |
| GT from N. aurantlaca | AVAIGILPLC ASSKQLPPPI MGLTPAKTLA GKAVHSFLRF |
| GT from F. solisilvae | VLSIGIAPLL ESSRDLAPYG LGLHPARSWA GKFRQAGLRW |
| GT from F. aestuarina | VVGVGVVPLT ESDDWVPPTS LGMKPQSGRV GRLVSRLLNY |
| GT from C. andamanensis | VIAVGIFPNI ASSRDLPPYG LGMRPASGFL GRKKQDLLRF |
| GT from A. macrocephali | IASIGVVPLA LSAPDLPLYG IGHQPATTFF GKRKQNFIKL |
| GT from F. sp. LCS9 | VITVGILPLP ETSKDLAPYG LAITPNYSFW GKKKQTFLRF |
| Chimera 1 | VVAVGVIPLA EESVDLAPYG TGLPPAATEE QRAMYFGMKD |
| Chimera 3 | VVAVGVIPLA EESVDLAPYG TGLPPAATEE QRAMYFGMKD |
| Chimera 4 | VVAVGVIPLA EESVDLAPYG TGLPPAATEE QRAMYFGMKD |
| SEQ ID NO. 1 | -------PLX ESXXXLPPXG XFXXPXXXXX GK-------- |
| alternate aa SEQ ID NO. 1 | A R |
| | ....\|....\| ....\|....\|.... \|....\| ....\|....\| |
| | 205 215 225 235 |
| GTC | VADLVLFRES NKVMRKMLTE HGIDHLYTN- VFDLMVKKST |
| GTD | ALANVVFKTA IDSFSAILDR YQVPHEKAI- LFDTLIRQSD |
| GTF | MTTRILFKPC DDLYNEIRQR YNMEPARDF- VFDSFIRTAD |
| GTS | IADELLFAQP TKVMWGLLAQ HGIDAGKAN- IFDILIQKST |
| GT from S. radiotolerans | MVQQVMFKPI NVLHNQLRQV YGLPPEPDS- VFDSIVRSAD |
| GT from N. aurantiaca | LTNKVLFKKP HALINEQYRR AGMLTNGKN- LFDLQIDKAT |
| GT from F. solisilvae | VADNILFRKS INVMYDLFEE YNIPHNGEN- FFDMGVRKAS |
| GT from F. aestuarina | LVQDVMLKPA NDLHNELRAQ YGLRPVPGF- IFDATVRQAD |
| GT from C. andamanensis | LTDKLVFGKQ NELNRQILRS WGIEAPGHLN LFDLQTQHAS |
| GT from A. macrocephali | MADKLIFDET KVVYNQLLRS LDLSEEENLT IFDIAPLQSD |
| GT from F. sp. LCS9 | VADQVLFRKP YLVMKEMLAD YGIKP-DGN- LFSTLIRKSS |
| Chimera 1 | ALANVVFKTA IDSFSAILDR YQVPHEKAI- LFDTLIRQSD |
| Chimera 3 | ALANVVFKTA IDSFSAILDR YQVPHEKAI- LFDTLIRQSD |
| Chimera 4 | ALANVVFKTA IDSFSAILDR YQVPHEKAI- LFDTLIRQSD |
| SEQ ID NO. 1 | ---------- ---------- ---------- ---------- |
| | ....\|....\| ....\|....\|.... \|....\| ....\|....\| |
| | 245 255 265 275 |
| GTC | LLLQSGTPGF EYYRSDLGKN IRFIGSLLPY QSKKQTT--- |
| GTD | LFLQIGAKAF EYDRSDLGEN VRFVGALLPY SESKSRQ--- |
| GTF | LYLQSGVPGF EYKRSKMSAN VRFVGPLLPY SSGIKPN--- |
| GTS | LVLQSGTPGF EYKRSDLSSH VHFIGPLLPY TKKKERE--- |
| GT from S. radiotolerans | VYLQSGVPSF EYPRKRISAN VQFVGPLLPY AKGQKHP--- |
| GT from N. aurantiaca | LFLQSCTPGF EYQRAHMSRH IHFIGPLLPS HSDAPAP--- |
| GT from F. solisilvae | LFLQSGTPGF EYNRSDLSEH IRFIGALLPY AGERKEE--- |
| GT from F. aestuarina | LYLQSGVPGF EFPRKRISPN VRFIGPMLPY SRANRQP--- |
| GT from C. andamanensis | VVLQNGTPGF EYTRSDLSPN LVFAGPLLPL VKKVRED--- |
| GT from A. macrocephali | VFLQNGIPEI DYPRYSLPES IKYVGALQVQ TNNNNNQKLK |
| GT from F. sp. LCS9 | LVLQSGTPGF EYFRSDLGHN IRFAGALLPY TTQKQTT--- |
| Chimera 1 | LFLQIGAKAF EYDRSDLGKN IRFIGSLLPY QSKKQTT--- |
| Chimera 3 | LFLQIGAKAF EYDRSDLGEN VRFVGALLPY SESKSRQ--- |
| Chimera 4 | LFLQIGAKAF EYDRSDLGEN VRFVGALLPY SESKSRQ--- |
| SEQ ID NO. 1 | --LQXGXPGF EYXR------ ---------- ---------- |
| alternate aa SEQ ID NO. 1 | C K D |
| | ....\|....\| ....\|....\|.... \|....\| ....\|....\| |
| | 285 295 305 315 |
| GTC | AWSDERLNRY EKIVVVTQGT VEKNIEKILV PTLEAFR-DT |
| GTD | PWFDQKLLQY GRIVLVTQGT VEHDINKILV PTLEAFK-NS |
| GTF | FAHAAKLKQY KKVILATQGT VERDPEKILV PTLEAFK-DT |
| GTS | SWYNEKLSHY DKVILVTQGT IEKDIEKLIV PTLEAFK-NS |
| GT from S. radiotolerans | FIQAKKALQY KKVILVTQGT IERDVQKIIV PTLEAFKNEP |
| GT from N. aurantlaca | FHFEDKLHQY AKVLLVTQGT FEGDVRKLIV PAIEAFK-NS |
| GT from F. solisilvae | PWFDSRLNKF DRVILVTQGT VERDVTKIIV PVLKAFR-DS |
| GT from F. aestuarina | FEQAIKTLAY KRVVLVTQGT VERNVEKIIV PTLEAYKKDP |
| GT from C. andamanensis | LPLQEKLRKY KNVILVTQGT AEQNTEKILA PTLEAFK-DS |
| GT from A. macrocephali | KDWSAILDTS KKIILVSQGT VEKNLDKLII PSLEAFK-DS |
| GT from F. sp. LCS9 | PWYNKKLEQY DKVILVTQGT VEKDVEKIIV PTLEAFK-DS |
| Chimera 1 | AWSDERLNRY EKIVVVTQGT VEKNIEKILV PTLEAFR-DT |
| Chimera 3 | PWFDQKLLQY GRIVLVTQGT VEHDINKILV PTLEAFK-NS |
| Chimera 4 | PWFDQKLLQY GQIVVVTQGT VEKNIEKILV PTLEAFR-DT |
| SEQ ID NO. 1 | ---------- ------TQGT XEKXXXKXXX PTLEAFR--- |
| alternate aa SEQ ID NO. 1 | R K |
| | ....\|....\| ....\|....\|.... \|....\| ....\|....\| |
| | 325 335 345 355 |
| GTC | DLLVIATTGG SGTAELKKRY PQ-GNLIIED FIPFGDIMPY |
| GTD | ETLVIATTGG NGTAELRARF PF-ENLIIED FIPFDDVMPR |
| GTF | DHLVVITTGG SKTAELRARY PQ-KNVIIED FIDFNLIMPH |
| GTS | DCLVIATTGG AYTEELRKRY PE-ENIIIED FIPFDDVMPY |
| GT from S. radiotolerans | TTLVIVTIGG SQTSELRARF PQ-ENFIIDD FIDFNAVMPY |
| GT from N. aurantiaca | RHLVVVTTAG WHTHKLRQRY KAFANVVIED FIPFSQIMPF |
| GT from F. solisilvae | NYLVVATTGG NGTKLLREQY KA-DNIIIED FIPFTDIMPY |
| GT from F. aestuarina | DILVIVTIGG SGTLALRKRY PQ-ANFIIED FIDFNAVMPY |
| GT from C. andamanensis | TWLVVATTGG AGTEALRARY PQ-ENFLIED YIPFDQIMPN |
| GT from A. macrocephali | DYIVLVATGY TDTKGLQKRY PQ-QHFYIED FIAYDAVMPH |
| GT from F. sp. LCS9 | DCLVVVTTGG SRTLELRLRY PQ-NNIIIED FIPFGDVMPY |
| Chimera 1 | DLLVIATTGG SGTAELKKRY PQ-GNLIIED FIPFGDIMPY |
| Chimera 3 | ETLVIATTGG NGTAELRARF PQ-GNLIIED FIPFGDIMPY |
| Chimera 4 | DLLVIATTGG SGTAELKKRY PQ-GNLIIED FIPFGDIMPY |
| SEQ ID NO. 1 | --LVXXTTGG ---------- -------IED FIPFXXVMPX |
| alternate aa SEQ ID NO. 1 | D I |
| | ....\|....\| ....\|....\|.... \|....\| ....\|....\| |
| | 365 375 385 395 |
| GTC | ADVYITNGGY GGVMLGIENQ LPLVVAGIHE GKNEINARIG |
| GTD | ADVYVTNGGY GGTLLSIHNQ LPMVAAGVHE GKNEVCSRIG |
| GTF | ADVYVTNSGF GGVMLSIQHG LPMVAAGVHE GKNEIAARIG |
| GTS | ADVYVSNGGY GGVLLSIQHQ LPMVVAGVHE GKNEINARVG |
| GT from S. radiotolerans | ASVYVTNGGY GGVMLALQHN LPIVVAGIHE GKNEIAARID |
| GT from N. aurantiaca | ADVFISNGGY GGVMQSISNK LPMVVAGIHE GKNEICARVG |
| GT from F. solisilvae | TDVYVTNGGY GGVMLGIENQ LPLVVAGVHE GKNEINARIG |
| GT from F. aestuarina | VSVYVTNGGY GGVMLALQHK LPIVAAGVHE GKNEIAARIG |
| GT from C. andamanensis | ADVYVSNGGF GGVLQAISHQ LPMVVAGVHE GKNEICARVG |
| GT from A. macrocephali | IDVFIMNGGY GSALLSIKHG VPMITAGVNE GKNEICSRMD |
| GT from F. sp. LCS9 | ADVYITNGGY GGVMLGIENQ LPMVVAGVHE GKNEICARVG |
| Chimera 1 | ADVYITNGGY GGVMLGIENQ LPLVVAGIHE GKNEINARIG |
| Chimera 3 | ADVYITNGGY GGVMLGIENQ LPLVVAGIHE GKNEINARIG |
| Chimera 4 | ADVYITNGGY GGVMLGIENQ LPLVVAGIHE GKNEINARIG |
| SEQ ID NO. 1 | XDVYITNGGY GGVMLXIXNX LPXVXAGXHE GKNE------ |
| alternate aa SEQ ID NO. 1 | FVS F L H |
| | ....\|....\| ....\|....\|.... \|....\| ....\|....\| |
| | 405 415 425 435 |
| GTC | YFELGINLKT EWPKPEQMKK AIDEVIGNKK YKENITKLAK |
| GTD | HFGCGINLET ETPTPDQIRE SVHKILSNDI FKKNVFRIST |
| GTF | YFKLGMNLKT ETPTPDQIRT SVETVLTDQT YRRNLARLRT |
| GTS | YFDLGINLKT ERPTVLQLRK SVDAVLQSDS YAKNVKRLGK |
| GT from S. radiotolerans | YCKVGIDLKT ETPSPTRIRH AVETVLTNDM YRQNVRQMGQ |
| GT from N. aurantiaca | YFKTGINMRT EHPKPEKIKT AVNEILSNPL YRKSVERLSK |
| GT from F. solisilvae | YFRLGIDLRN ERPTPEQMRN AIEKVIANGE YRRNVQALAR |
| GT from F. aestuarina | YCQVGVDLRT ETPTPDQIRR AVATILGDET YRRQVRRLSD |
| GT from C. andamanensis | YFKLGLDLKT ETPKPAQIRA AVEQVLQDPQ YRHKVQALSA |
| GT from A. macrocephali | YSGVGIDLKT EKPRAVTIQN ATERILGTDK YLDTIQKIQQ |
| GT from F. sp. LCS9 | YFQLGINLKT EQPIPAQIRN SVEEILSNVV YKKNVVKLSK |
| Chimera 1 | YFELGINLKT EWPKPEQMKK AIDEVIGNKK YKENITKLAK |
| Chimera 3 | YFELGINLKT EWPKPEQMKK AIDEVIGNKK YKENITKLAK |
| Chimera 4 | YFELGINLKT EWPKPEQMKK AIDEVIGNKK YKENITKLAK |
| SEQ ID NO. 1 | ---------- ---------- ---------- ---------- |
| | ....\|....\| ....\|....\|.... \|....\| ....\|....\| |
| | 445 455 465 475 |
| GTC | EFSNYHPNEL CAQYISEVLQ KTGRLYISSK KEEEKIY--- |
| GTD | HLD-VDANEK SAGHILDLLE ERVVCG---- ---------- |
| GTF | EFAQYDPMAL SERYINELLA KQPRKQHEAV EAI------- |
| GTS | EFKQYDPNEI CEKYVAQLLE NQISYKEKAN SYQAEVLV-- |
| GT from S. radiotolerans | EFSQYQPTEL AEQYINALLI QEKSSRLAVV A--------- |
| GT from N. aurantiaca | EFSEYDPLAL CEKFVNALPV LQKP------ ---------- |
| GT from F. solisilvae | EFKTYAPLEL TERFVTELLL SRRHKLVPVN DDALIY---- |
| GT from F. aestuarina | EFGRYNPNQL AEQYINELLA QSVGEPVAAL S--------- |
| GT from C. andamanensis | EFRQYNPQQL CEHWVQRLTG GRRAAAPAPQ SAGGQLLSLT |
| GT from A. macrocepha1i | RMNSYNTLDI CEQHISRLIS E--------- ---------- |
| GT from F. sp. LCS9 | EFAQYKPNEL CAKYVAQLVQ -QESSSQKVN VAAVEAVLEA |
| Chimera 1 | EFSNYHPNEL CAQYISEVLQ KQAG-FISAV KRKKKRYTKD |
| Chimera 3 | EFSNYHPNEL CAQYISEVLQ KTGRLYISSK KEEEKIY--- |
| Chimera 4 | EFSNYHPNEL CAQYISEVLQ KTGRLYISSK KEEEKIY--- |
| SEQ ID NO. 1 | ---------- ---------- ---------- ---------- |
| | ....\|....\| |
| | 485 |
| GTC | ---------- |
| GTD | ---------- |
| GTF | ---------- |
| GTS | ---------- |
| GT from S. radiotolerans | ---------- |
| GT from N. aurantiaca | ---------- |
| GT from F. solisilvae | ---------- |
| GT from F. aestuarina | ---------- |
| GT from C. andamanensis | LN-------- |
| GT from A. macrocephali | ---------- |
| GT from F. sp. LCS9 | ---------- |
| Chimera 1 | PAANKARKEA |
| Chimera 3 | ---------- |
| Chimera 4 | ---------- |
| SEQ ID NO. 1 | ---------- |

Accordingly, in the methods of the present invention, it is preferred that a glycosyl transferase comprising some or preferably all of the above conserved amino acids/sequence motifs is used as long as the glycosyl transferase maintains its desired function of rhamnosylating flavonoids at position R3 of formula (I). These amino acids/sequence motifs are comprised in SEQ ID NO:1. Thus, in one preferred embodiment of the present invention, a glycosyl transferase is used, which comprises the amino acid sequence of SEQ ID NO:1 and which shows the desired activity of rhamnosylating flavonoids at position R3 of Formula (I) as shown above, corresponding to 5-O rhamnosylation of flavonoids. The invention furthermore relates to a method for rhamnosylation of flavonoids using a glycosyl transferase comprising an amino acid sequence of the known glycosyl transferases GTC, GTD, GTF or related enzymes from Segetibacter koreensis, Flavihumibacter solisilvae, Cesiribacter andamanensis, Niabella aurantiaca, Spirosoma radiotolerans, Fibrella aestuarina, or Aquimarina macrocephali. Accordingly, in one embodiment, a glycosyl transferase having the amino acid sequence as shown in any one of SEQ ID NOs: 3, 5, 7, 9, 11, 13, 15, 17, 19, 21, 23, 25, 56, 58, or 61 is used in the methods of the present invention. In this regard, the skilled person is well-aware that these sequences may be altered without altering the function of the polypeptide. For example, it is known that enzymes such as glycosyl transferases generally possess an active site responsible for the enzymatic activity. Amino acids outside of the active site or even within the active site may be altered while the enzyme in its entirety maintains a similar or identical activity. It is known that enzymatic activity may even be increased by alterations to the amino acid sequence. Therefore, in the methods of the present invention, glycosyl transferases may be used comprising an amino acid sequence having at least 80, 85, 90, 91, 92, 93, 94, 95, 96, 97, 98, 99 or 100% sequence identity with SEQ ID NOs: 3, 5, 7, 9, 11, 13, 15, 17, 19, 21, 23, 25, 56, 58, or 61, respectively, as long as the function of rhamnosylating flavonoids at position R3 of Formula (I) is maintained. Methods how to test this activity are described herein and/or are known to the person skilled in the art.

In the methods of the present invention, glycosyl transferases may be used that are encoded by a polynucleotide comprising the nucleic acid sequences encoding the above glycosyl transferases. In particular, a glycosyl transferase encoded by a polynucleotide comprising any of the nucleic acid sequences of SEQ ID NOs: 2, 4, 6, 8, 10, 12, 14, 16, 18, 20, 22, 24, 26, 27, 28, 29, 30, 31, 32, 33, 34, 35, 36, 37, 38, 57, 59, 60, 62, or 63 may be used. As is known in the art, the genetic code is degenerated, which allows alterations to the sequence of nucleic acids comprised in a polynucleotide without altering the polypeptide encoded by the polynucleotide. Accordingly, in the methods of the present invention, glycosyl transferases may be used that are encoded by a polynucleotide having at least 80, 85, 90, 91, 92, 93, 94, 95, 96, 97, 98, 99 or 100% sequence identity with SEQ ID NOs: 2, 4, 6, 8, 10, 12, 14, 16, 18, 20, 22, 24, 26, 27, 28, 29, 30, 31, 32, 33, 34, 35, 36, 37, 38, 57, 59, 60, 62, or 63. Because further alterations to the polynucleotide may be made without altering the structure/function of the encoded polypeptide, glycosyl transferases may be used in the methods of the present invention that are encoded by a polynucleotide hybridizable under stringent conditions with a polynucleotide comprising SEQ ID NOs: 2, 4, 6, 8, 10, 12, 14, 16, 18, 20, 22, 24, 26, 27, 28, 29, 30, 31, 32, 33, 34, 35, 36, 37, 38, 57, 59, 60, 62, or 63.

Within the meaning of the present invention, the term “polypeptide” or “enzyme” refers to amino acids joined to each other by peptide bonds or modified peptide bonds, i.e., peptide isosteres, and may contain modified amino acids other than the 20 gene-encoded amino acids. The polypeptides may be modified by either natural processes, such as post-translational processing, or by chemical modification techniques which are well known in the art. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications can include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and/or transfer-RNA mediated addition of amino acids to protein such as arginylation. (See Proteins Structure and Molecular Properties 2nd Ed., T. E. Creighton, W. H. Freeman and Company, New York (1993); Posttranslational Covalent Modification of Proteins, B. C. Johnson, Ed., Academic Press, New York, pp. 1-12 (1983)).

While the glycosyl transferase used in the methods of the present invention may be contacted/incubated with a flavonoid directly, it is preferred that the method further comprises a step of providing a host cell transformed with a gene encoding said glycosyl transferase. As such, the glycosyl transferase is recombinantly expressed by the host cell and provided by the host cell for being contacted/incubated with the flavonoid. It is preferred that the host cell is incubated prior to contacting/incubating said host cell with a flavonoid. That is, it is preferred that the host cell is allowed to recombinantly express the glycosyl transferase prior to addition of a flavonoid for production of a rhamnosylated version thereof.

The type of host cell is not particularly limited. In principle, any cell may be used as host cell to recombinantly express a glycosyl transferase. For example, the organism may be used from which the glycosyl transferase gene is derived. However, it is preferred in the methods of the present invention that the host cell is a prokaryotic host cell.

As used herein, “prokaryote” and “prokaryotic host cell” refer to cells which do not contain a nucleus and whose chromosomal material is thus not separated from the cytoplasm. Prokaryotes include, for example, bacteria. Prokaryotic host cells particularly embraced by the present invention include those amenable to genetic manipulation and growth in culture. Exemplary prokaryotes routinely used in recombinant protein expression include, but are not limited to, E. coli, Bacillus lichenifauuis (van Leen, et al. (1991) Bio/Technology 9:47-52), Ralstonia eutropha (Srinivasan, et al. (2002) Appl. Environ. Microbiol. 68:5925-5932), Methylobacterium extorquens (Belanger, et al. (2004) FEMS Microbiol Lett. 231 (2): 197-204), Lactococcus lactic (Oddone, et al. (2009) Plasmid 62(2): 108-18) and Pseudomonas sp. (e.g., P. aerugenosa, P. fluorescens and P. syringae). Prokaryotic host cells can be obtained from commercial sources (e.g., Clontech, Invitrogen, Stratagene and the like) or repositories such as American Type Culture Collection (Manassas, Va.).

In the methods of the present invention, it is preferred that the prokaryotic host cell, in particular the bacterial host cell, is E. coli. The expression of recombinant proteins in E. coli is well-known in the art. Protocols for E. coli-based expression systems are found in Sambrook “Molecular Cloning” Cold Spring Harbor Laboratory Press 2012.

The host cells of the invention are recombinant in the sense that they have been genetically modified for the purposes of harboring polynucleotides encoding a glycosyl transferase. Generally, this is achieved by isolating nucleic acid molecules encoding the protein or peptide of interest and introducing the isolated nucleic acid molecules into a prokaryotic cell.

Nucleic acid molecules encoding the proteins of interest, i.e. a glycosyl transferase, can be isolated using any conventional method. For example, the nucleic acid molecules encoding the glycosyl transferase may be obtained as restriction fragments or, alternatively, obtained as polymerase chain reaction amplification products. Techniques for isolating nucleic acid molecules encoding proteins such as glycosyl transferases are routinely practiced in the art and discussed in conventional laboratory manuals such as Sambrook and Russell (Molecular Cloning: A Laboratory Manual, 4th Edition, Cold Spring Harbor Laboratory press (2012)) and Ausubel et al. (Short Protocols in Molecular Biology, 52nd edition, Current Protocols (2002)).

To facilitate the expression of proteins (including enzymes) or peptides in the prokaryotic host cell, in particular the glycosyl transferase, the isolated nucleic acid molecules encoding the proteins or peptides of interest are incorporated into one or more expression vectors. Expression vectors compatible with various prokaryotic host cells are well-known and described in the art cited herein. Expression vectors typically contain suitable elements for cloning, transcription and translation of nucleic acids. Such elements include, e.g., in the 5′ to 3′ direction, a promoter (unidirectional or bidirectional), a multiple cloning site to operatively associate the nucleic acid molecule of interest with the promoter, and, optionally, a termination sequence including a stop signal for RNA polymerase and a polyadenylation signal for polyadenylase. In addition to regulatory control sequences discussed herein, the expression vector can contain additional nucleotide sequences. For example, the expression vector can encode a selectable marker gene to identify host cells that have incorporated the vector. Nucleic acids encoding a selectable marker can be introduced into a host cell on the same vector as that containing the nucleic acid of interest or can be introduced on a separate vector. Cells stably transfected with the introduced nucleic acid can be identified by drug selection (e.g., cells that have incorporated the selectable marker gene will survive, while the other cells die). Expression vectors can be obtained from commercial sources or be produced from plasmids routinely used in recombinant protein expression in prokaryotic host cells. Exemplary expression vectors include, but are not limited to pBR322, which is the basic plasmid modified for expression of heterologous DNA in E. coli; RSF1010 (Wood, et al. (1981) J. Bacteriol. 14:1448); pET3 (Agilent Technologies); pALEX2 vectors (Dualsystems Biotech AG); and pET100 (Invitrogen).

The regulatory sequences employed in the expression vector may be dependent upon a number of factors including whether the protein of interest, i.e. the glycosyl transferases, is to be constitutively expressed or expressed under inducible conditions (e.g., by an external stimulus such as IPTG). In addition, proteins expressed by the prokaryotic host cell may be tagged {e.g., his6-, FLAG- or GST-tagged) to facilitate detection, isolation and/or purification.

Vectors can be introduced into prokaryotic host cells via conventional transformation techniques. Such methods include, but are not limited to, calcium chloride (Cohen, et al. (1972) Proc. Natl. Acad. Sci. USA 69:2110-2114; Hanahan (1983) J. Mol. Biol. 166:557-580; Mandel & Higa (1970) J. Mol. Biol. 53:159-162), electroporation (Shigekawa & Dower (1988) Biotechniques 6:742-751), and those described in Sambrook et al. (2012), supra. For a review of laboratory protocols on microbial transformation and expression systems, see Saunders & Saunders (1987) Microbial Genetics Applied to Biotechnology Principles and Techniques of Gene Transfer and Manipulation, Croom Helm, London; Puhler (1993) Genetic Engineering of Microorganisms, Weinheim, N.Y.; Lee, et al. (1999) Metabolic Engineering, Marcel Dekker, NY; Adolph (1996) Microbial Genome Methods, CRC Press, Boca Raton; and Birren & Lai (1996) Nonmammalian Genomic Analysis: A Practical Guide, Academic Press, San Diego.

As an alternative to expression vectors, it is also contemplated that nucleic acids encoding the proteins (including enzymes) and peptides disclosed herein can be introduced by gene targeting or homologous recombination into a particular genomic site of the prokaryotic host cell so that said nucleic acids are stably integrated into the host genome.

Recombinant prokaryotic host cells harboring nucleic acids encoding a glycosyl transferase can be identified by conventional methods such as selectable marker expression, PCR amplification of said nucleic acids, and/or activity assays for detecting the expression of the glycosyl transferase. Once identified, recombinant prokaryotic host cells can be cultured and/or stored according to routine practices.

With regards to culture methods of recombinant host cells, the person skilled in the art is well-aware how to select and optimize suitable methods for efficient culturing of such cells.

As used herein, the terms “culturing” and the like refer to methods and techniques employed to generate and maintain a population of host cells capable of producing a recombinant protein of interest, in particular the glycosyl transferase, as well as the methods and techniques for optimizing the production of the protein of interest, i.e. the glycosyl transferase. For example, once an expression vector has been incorporated into an appropriate host, preferably E. coli, the host can be maintained under conditions suitable for high level expression of the relevant polynucleotide. When using the methods of the present invention, the protein of interest, i.e. the glycosyl transferase, may be secreted into the medium. Where the protein of interest is secreted into the medium, supernatants from such expression systems can be first concentrated using a commercially available protein concentration filter, e.g., an Amicon™ or Millipore Pellicon™ ultrafiltration unit, which can then be subjected to one or more additional purification techniques, including but not limited to affinity chromatography, including protein A affinity chromatography, ion exchange chromatography, such as anion or cation exchange chromatography, and hydrophobic interaction chromatography.

Culture media used for various recombinant host cells are well known in the art. Generally, a growth medium or culture medium is a liquid or gel designed to support the growth of microorganisms or cells. There are different types of media for growing different types of cells.

Culture media used to culture recombinant bacterial cells will depend on the identity of the bacteria. Culture media generally comprise inorganic salts and compounds, amino acids, carbohydrates, vitamins and other compounds that are either necessary for the growth of the host cells or improve health or growth or both of the host cells. In particular, culture media typically comprise manganese (Mn²⁺) and magnesium (Mg²⁺) ions, which are co-factors for many, but not all, glycosyltransferases. The most common growth/culture media for microorganisms is LB medium (Lysogeny Broth). LB is a nutrient medium.

Nutrient media contain all the elements that most bacteria need for growth and are non-selective, so they are used for the general cultivation and maintenance of bacteria kept in laboratory culture collections.

In this regard, an undefined medium (also known as a basal or complex medium) is a medium that contains: a carbon source such as glucose for bacterial growth, water, various salts needed for bacterial growth, a source of amino acids and nitrogen (e.g., beef, yeast extract). In contrast, a defined medium (also known as chemically defined medium or synthetic medium) is a medium in which all the chemicals used are known and no yeast, animal or plant tissue is present. In the methods of the present invention, either defined or undefined nutrient media may be used. However, it is preferred that lysogeny broth (LB) medium, terrific broth (TB) medium, Rich Medium (RM), Standard I medium or a mixture thereof be used in the methods of the present invention.

Alternatively, minimal media may be used in the methods of the present invention. Minimal media are those that contain the minimum nutrients possible for colony growth, generally without the presence of amino acids. Minimal medium typically contains a carbon source for bacterial growth, which may be a sugar such as glucose, or a less energy-rich source like succinate, various salts, which may vary among bacteria species and growing conditions; these generally provide essential elements such as magnesium, nitrogen, phosphorus, and sulfur to allow the bacteria to synthesize protein and nucleic acid and water. Supplementary minimal media are a type of minimal media that also contains a single selected agent, usually an amino acid or a sugar. This supplementation allows for the culturing of specific lines of auxotrophic recombinants. Accordingly, in one embodiment the methods of the present invention are done in minimal medium. Preferably, the minimal medium is a mineral salt medium (MSM) or M9 medium supplemented with a carbon source and an energy source, preferably wherein said carbon and energy sources are glycerol, glucose, maltose, sucrose, starch and/or molasses.

Media used in the methods of the present invention are prepared following methods well-known in the art. In this regard, a method for preparing culture medium generally comprises the preparation of a “base medium”. The term “base medium” or broth refers to a partial broth comprising certain basic required components readily recognized by those skilled in the art, and whose detailed composition may be varied while still permitting the growth of the microorganisms to be cultured. Thus in embodiments and without limitation, base medium may comprise salts, buffer, and protein extract, and in embodiments may comprise sodium chloride, monobasic and dibasic sodium phosphate, magnesium sulphate and calcium chloride. In embodiments a liter of core medium may have the general recipe known in the art for the respective medium, but in alternative embodiments core media will or may comprise one or more of water, agar, proteins, amino acids, caesein hydrolysate, salts, lipids, carbohydrates, salts, minerals, and pH buffers and may contain extracts such as meat extract, yeast extract, tryptone, phytone, peptone, and malt extract, and in embodiments medium may be or may comprise luria bertani (LB) medium; low salt LB medium (1% peptone, 0.5% yeast extract, and 0.5% NaCl), SOB medium (2% peptone, 0.5% Yeast extract, 10 mM NaCl, 2.5 mM KCl, 10 mM MgCl₂, 10 mM MgSO₄), SOC medium (2% peptone, 0.5% Yeast extract, 10 mM NaCl, 2.5 mM KCl, 10 mM MgCl₂, 10 mM MgSO₄, 20 mM Glucose), Superbroth (3.2% peptone, 2% yeast extract, and 0.5% NaCl), 2×TY medium (1.6% peptone, 1% yeast extract, and 0.5% NaCl), TerrificBroth (TB) (1.2% peptone, 2.4% yeast extract, 72 mM K2HPO4, 17 mM KH2PO4, and 0.4% glycerol), LB Miller broth or LB Lennox broth (1% peptone, 0.5% yeast extract, and 1% NaCl). It will be understood that in particular embodiments one or more components may be omitted from the base medium.

In the methods of the present invention, the host cell may be cultured in the medium prior to incubating/contacting the host cell with an agent for inducing expression of the foreign gene, i.e. the glycosyl transferase, and prior to addition of the flavonoid to be bioconverted. Alternatively, the flavonoid may be added to the culture together with the host cell, thus, prior to amplifying the number of host cells in the culture medium.

The person skilled in the art will readily understand that the growth of a desired microorganism, in particular E. coli, will be best promoted at selected temperatures suited to the microorganism in question. In particular embodiments culturing may be carried out at about 28° C. and the broth to be used may be pre-warmed to this temperature preparatory to inoculation with a sample for testing. However, in the methods of the present invention culturing may be carried out at any temperature suitable for the desired purpose, i.e. the production of a rhamnosylated flavonoid. However, it is preferred that culturing is done at a temperature between about 20° C. and about 37° C. That is, culturing is preferably done at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C. or about 37° C. More preferably, culturing may be carried out at a temperature between about 24° C. to about 30° C. Most preferably, culturing in the methods of the present invention is done at a temperature of about 28° C.

Similarly, contacting/incubating the cultured host cell with a flavonoid may be done at any temperature suitable for efficient production of a rhamnosylated flavonoid. Preferably, the temperature for culturing the host cell and the temperature for contacting/incubating the host cell and the glycosyl transferase with a flavonoid are about identical. That is, it is preferred that contacting/incubating the host cell and the expressed glycosyl transferase with a flavonoid is done at a temperature between about 20° C. and about 37° C. Contacting/incubating the host cell and the expressed glycosyl transferase with a flavonoid is preferably done at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C. or about 37° C. More preferably, contacting/incubating the host cell and the expressed glycosyl transferase with a flavonoid may be carried out at a temperature between about 24° C. to about 30° C. Most preferably, contacting/incubating the host cell and the expressed glycosyl transferase with a flavonoid in the methods of the present invention is done at a temperature of about 28° C.

In the methods of the present invention, the pH of culture medium is generally set at between about 6.5 and about 8.5 and for example in particular embodiments is or is about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5 or may be in ranges delimited by any two of the foregoing values. Thus, in particular embodiments the pH of culture medium is in ranges with lower limits of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, or 8.4 and with upper limits of about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. In a preferred embodiment the culture medium has a pH between about 7.0 and 8.0. In a more preferred embodiment of the present invention, the medium has a pH of about 7.4. However, it will be understood that a pH outside of the range pH 6.5-8.5 may still be useable in the methods of the present invention, but that the efficiency and selectivity of the culture may be adversely affected.

A culture may be grown for any desired period following inoculation with a recombinant host cell, but it has been found that a 3 hour culture period above 20° C. and starting from an optical density (OD) of 0.1 at 600 nm is sufficient to enrich the content of E. coli sufficiently to permit efficient expression of the glycosyl transferase and subsequent contacting/incubating with the flavonoid for successful bioconversion. However, the culture period may be longer or shorter and may be up to or less than 1, 2, 3, 4, 5, 6, 7, 8, 9, 10, 11, 12, 13, 14 or more hours. Those skilled in the art will readily select a suitable culture period to satisfy particular requirements.

In the methods of the present invention, the culture medium may be further enriched/supplemented. That is, it is preferred that during culturing of the host cell and/or during contacting/incubating the host cell and the expressed glycosyl transferase with a flavonoid, the concentration of dissolved oxygen (DO) is monitored and maintained at a desired value. Preferably, in the methods of the present invention, the concentration of dissolved oxygen (DO) is maintained at about 30% to about 50%. Moreover, when the concentration of dissolved oxygen is above about 50%, a nutrient may be added, preferably wherein the nutrient is glucose, sucrose, maltose or glycerol. That is, the medium may be supplemented/enriched during culturing/contacting/incubating to maintain conditions that allow efficient production of the glycosyl transferase and/or efficient bioconversion of the flavonoid.

In one embodiment, the methods of the present invention may be done as fed-batch culture or semi-batch culture. These terms are used interchangeably to refer to an operational technique in biotechnological processes where one or more nutrients (substrates) are fed (supplied) to the bioreactor during cultivation and in which the product(s) remain in the bioreactor until the end of the run. In some embodiments, all the nutrients are fed into the bioreactor.

In the methods of the present invention, a step of harvesting the incubated host cell prior to contacting/incubating said host cell with a flavonoid may be added. That is, the methods of the present invention may comprise culturing the host cell in a culture medium until a desired optical density (OD) and harvesting the host cell when the desired OD is reached. The OD may be between about 0.6 and 1.0, preferably about 0.8. Expression of the glycosyl transferase may either be induced prior to harvesting or subsequently to harvesting, for example together with addition of the flavonoid. The culture medium may be changed subsequently to harvesting or the host cell may be resuspended in culture medium used for growth of the host cell. That is, in one embodiment, methods of the present invention further comprise solubilization of the harvested host cell in a buffer prior to contacting/incubating said host cell with a flavonoid, preferably wherein the buffer is phosphate-buffered saline (PBS), preferably supplemented with a carbon and energy source, preferably glycerol, glucose, maltose, and/or sucrose, and growth additives, preferably vitamins including biotin and/or thiamin.

In the methods of the present invention, harvesting may be done using any method suitable for that purpose. It is preferred that harvesting is done using a membrane filtration method, preferably a hollow fibre membrane device, or centrifugation.

In the methods of the present invention, the flavonoid to be rhamnosylated is not particularly limited as long as the flavonoid belongs to the class of flavonoids as known in the art and, as such, is a member of a group of compounds widely distributed in plants, fulfilling many functions. Flavonoids are the most important plant pigments for flower coloration, producing yellow or red/blue pigmentation in petals designed to attract pollinator animals. In higher plants, flavonoids are involved in UV filtration, symbiotic nitrogen fixation and floral pigmentation.

As such, the flavonoid preferably is a flavanone, flavone, isoflavone, flavonol, flavanonol, chalcone, flavanol, anthocyanidine, aurone, flavan, chromene, chromone or xanthone. Within the meaning of the present invention, the latter three are comprised in this class. As such, the term “flavonoid” refers to any compounds falling under the general formula (I) and is thus not limited to compounds which are generally considered flavonoid-type compounds.

It is preferred that the flavonoid used in the methods of the present invention is a compound or a solvate of the following Formula (I)

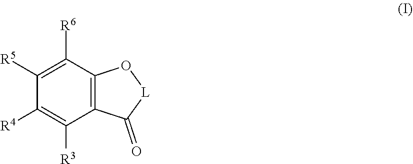

wherein: [symbol]
is a double bond or a single bond;

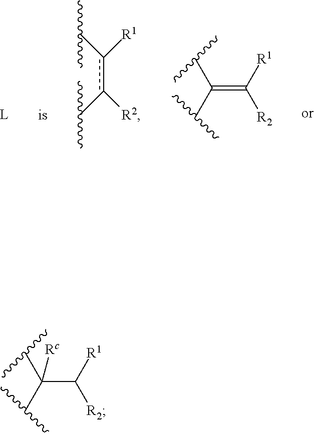

R¹ and R² are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from OH;
or R¹ and R² are joined together to form, together with the carbon atom(s) that they are attached to, a carbocyclic or heterocyclic ring being optionally substituted with one or more substituents R^{e}; wherein each R^{e} is independently selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};

R⁴, R⁵ and R⁶ are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};

or alternatively, R⁴ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and
R⁵ and R⁶ are joined together to form, together with the carbon atoms that they are attached to, a carbocyclic or heterocyclic ring being optionally substituted with one or more substituents R^{c};
or alternatively, R⁴ and R⁵ are joined together to form, together with the carbon atoms that they are attached to, a carbocyclic or heterocyclic ring being optionally substituted with one or more substituents R^{c}; and
R⁶ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
each R^{a} is independently selected from a single bond, C₁₋₅ alkylene, C₂₋₅ alkenylene, arylene and heteroarylene; wherein said alkylene, said alkenylene, said arylene and said heteroarylene are each optionally substituted with one or more groups R^{c};
each R^{b} is independently selected from hydrogen, C₁₋₅ alkyl, C₂₋₅ alkenyl, C₂₋₅ alkynyl, heteroalkyl, cycloalkyl, heterocycloalkyl, aryl and heteroaryl; wherein said alkyl, said alkenyl, said alkynyl, said heteroalkyl, said cycloalkyl, said heterocycloalkyl, said aryl and said heteroaryl are each optionally substituted with one or more groups R^{c};
each R^{c} is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₁₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl;
each R^{d} is independently selected from a monosaccharide, a disaccharide and an oligosaccharide; and
R³ is rhamnoslyated by the method of the present invention.

In this regard, rhamnosylating/rhamnosylation preferably is the addition of —O-(rhamnosyl) at position R³ of Formula (I) as shown above, wherein said rhamnosyl is substituted at one or more of its —OH groups with one or more groups independently selected from C₁₋₅ alkyl, C₂₋₅ alkenyl, C₂₋₅ alkynyl, a monosaccharide, a disaccharide and an oligosaccharide.

As used herein, the term “hydrocarbon group” refers to a group consisting of carbon atoms and hydrogen atoms. Examples of this group are alkyl, alkenyl, alkynyl, alkylene, carbocyl and aryl. Both monovalent and divalent groups are encompassed.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more carbon-to-carbon double bonds. The teen “C₂₋₅ alkynyl” denotes an alkynyl group having 2 to 5 carbon atoms. Preferred exemplary alkynyl groups are ethynyl, propynyl, or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the teen “C₀₋₃ alkylene” indicates that a covalent bond (corresponding to the option “Co alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. Unless defined otherwise, “heterocyclyl” preferably refers to heteroaryl, heterocycloalkyl or heterocycloalkenyl.

As used herein, the term “heterocyclic ring” refers to saturated or unsaturated rings containing one or more heteroatoms, preferably selected from oxygen, nitrogen and sulfur. Examples include heteroaryl and heterocycloalkyl as defined herein. Preferred examples contain, 5 or 6 atoms, particular examples, are 1,4-dioxane, pyrrole and pyridine.

The term “carbocyclic ring” means saturated or unsaturated carbon rings such as aryl or cycloalkyl, preferably containing 5 or 6 carbon atoms. Examples include aryl and cycloalkyl as defined herein.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), anthracenyl, or phenanthrenyl. Unless defined otherwise, an “aryl” preferably has 6 to 14 ring atoms, more preferably 6 to 10 ring atoms, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromenyl, xanthenyl, phenoxathiinyl, pyrrolyl (e.g., 2H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, pteridinyl, carbazolyl, beta-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, furazanyl, phenoxazinyl, pyrazolo[1,5-a]pyrimidinyl (e.g., pyrazolo[1,5-a]pyrimidin-3-yl), 1,2-benzisoxazol-3-yl, benzothiazolyl, benzoxazolyl, benzisoxazolyl, benzimidazolyl, 1H-tetrazolyl, 2H-tetrazolyl, coumarinyl, or chromonyl. Unless defined otherwise, a “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

The term “heteroalkyl” refers to saturated linear or branched-chain monovalent hydrocarbon radical of one to twelve carbon atoms, including from one to six carbon atoms and from one to four carbon atoms, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkyl radical may be optionally substituted independently with one or more substituents described herein. The term “heteroalkyl” encompasses alkoxy and heteroalkoxy radicals.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heterocycloalkyl” may, e.g., refer to oxetanyl, tetrahydrofuranyl, piperidinyl, piperazinyl, aziridinyl, azetidinyl, pyrrolidinyl, imidazolidinyl, morpholinyl (e.g., morpholin-4-yl), pyrazolidinyl, tetrahydrothienyl, octahydroquinolinyl, octahydroisoquinolinyl, oxazolidinyl, isoxazolidinyl, azepanyl, diazepanyl, oxazepanyl or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂.

As used herein, the term “rhamnosyl” refers to a substituted or unsubstituted rhamnose residue which is preferably connected via the C1-OH group of the same.

The term “monosaccharide” as used herein refers to sugars which consist of only a single sugar unit. These include all compounds which are commonly referred to as sugars and includes sugar alcohols and amino sugars. Examples include tetroses, pentoses, hexoses and heptoses, in particular aldotetroses, aldopentoses, aldohexoses and aldoheptoses.

Aldotetroses include erythrose and threose and the ketotetroses include erythrulose.

Aldopentoses include apiose, ribose, arabinose, lyxose, and xylose and the ketopentoses include ribulose and xylulose. The sugar alcohols which originate in pentoses are called pentitols and include arabitol, xylitol, and adonitol. The saccharic acids include xylosaccharic acid, ribosaccharic acid, and arabosaccharic acid.

Aldohexoses include galactose, talose, altrose, allose, glucose, idose, mannose, rhamnose, fucose, olivose, rhodinose, and gulose and the ketohexoses include tagatose, psicose, sorbose, and fructose. The hexitols which are sugar alcohols of hexose include talitol, sorbitol, mannitol, iditol, allodulcitol, and dulcitol. The saccharic acids of hexose include mannosaccharic acid, glucosaccharic acid, idosaccharic acid, talomucic acid, alomucic acid, and mucic acid.

Examples of aldoheptoses are idoheptose, galactoheptose, mannoheptose, glucoheptose, and taloheptose. The ketoheptoses include alloheptulose, mannoheptulose, sedoheptulose, and taloheptulose.

Examples of amino sugars are fucosamine, galactosamine, glucosamine, sialic acid, N-acetylglucosamine, and N-acetylgalactosamine.

As used herein, the term “disaccharide” refers to a group which consists of two monosaccharide units. Disaccharides may be formed by reacting two monosaccharides in a condensation reaction which involves the elimination of a small molecule, such as water.

Examples of disaccharides are maltose, isomaltose, lactose, nigerose, sambubiose, sophorose, trehalose, saccharose, rutinose, and neohesperidose.

As used herein, the term “oligosaccharide” refers to a group which consists of three to eight monosaccharide units. Oligosaccharide may be formed by reacting three to eight monosaccharides in a condensation reaction which involves the elimination of a small molecule, such as water. The oligosaccharides may be linear or branched.

Examples are dextrins as maltotriose, maltotetraose, maltopentaose, maltohexaose, maltoheptaose, and maltooctaose, fructo-oligosaccharides as kestose, nystose, fructosylnystose, bifurcose, inulobiose, inulotriose, and inulotetraose, galacto-oligosaccharides, or mannan-oligosaccharides.

As used herein, the expression “the compound contains at least one OH group in addition to any OH groups in R³” indicates that there is at least one OH group in the compound at a position other than residue R³. Examples of the OH groups in R³ are OH groups of the rhamnosyl group or of any substituents thereof. Consequently, for the purpose of determining whether the above expression is fulfilled, the residue R³ is disregarded and the number of the remaining OH groups in the compound is determined.

As used herein, the expression “an OH group directly linked to a carbon atom being linked to a neighboring carbon or nitrogen atom via a double bond” indicates a group of the following partial structure:

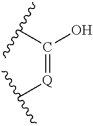

in which Q is N or C which may be further substituted. The double bond between C and Q may be part of a larger aromatic system and may thus be delocalized. Examples of such OH groups include OH groups which are directly attached to aromatic moieties, such as, aryl or heteroaryl groups. One specific example is a phenolic OH group.

As used herein, the term “substituted at one or more of its —OH groups” indicates that a substituent may be attached to one or more of the “—OH” groups in such a manner that the resulting group may be represented by “—O-substituent”.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

When specific positions in the compounds of formula (I) or formula (II) are referred to, the positions are designated as follows:

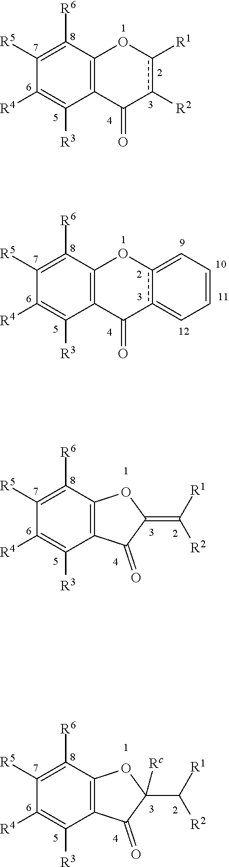

A skilled person will appreciate that the substituent groups comprised in the compounds of formula (I) may be attached to the remainder of the respective compound via a number of different positions of the corresponding specific substituent group. Unless defined otherwise, the preferred attachment positions for the various specific substituent groups are as illustrated in the examples.

As used herein, the term “about” preferably refers to ±10% of the indicated numerical value, more preferably to ±5% of the indicated numerical value, and in particular to the exact numerical value indicated.

Accordingly, it is preferred that a compound of the following formula (I) or a solvate thereof is used in the methods of the present invention as starting compound

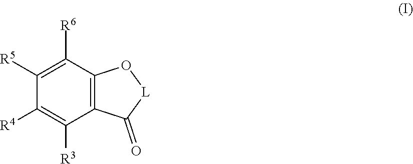

Many specific examples of the compound of following formula (I) are disclosed herein, such as, compounds of formulae (II), (IIa), (IIb), (IIc), (IId), (III) and (IV). It is to be understood that, if reference is made to the compound of formula (I), this reference also includes any of the compounds of formulae (II), (IIa), (IIb), (IIc), (IId), (III), (IV) etc.

In the present invention, the sign [symbol] represents a double bond or a single bond. In some examples, the sign [symbol] represents a single bond. In other examples, the sign [symbol] represents a double bond.

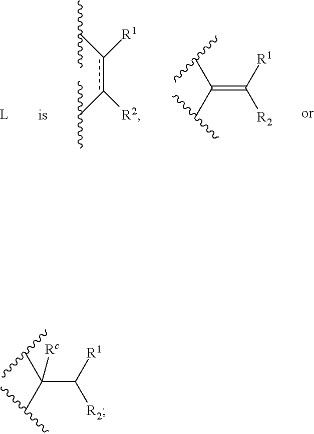

It is preferred that L be

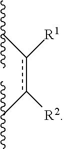

In preferred compounds of formula (I), R¹ and R² are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from —OH.

In preferred compounds of formula (I), R¹ is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}. In more preferred compounds of formula (I), R¹ is selected from cycloalkyl, heterocycloalkyl, aryl and heteroaryl; wherein said cycloalkyl, said heterocycloalkyl, said aryl and said heteroaryl are each optionally substituted with one or more groups R^{c}. In even more preferred compounds of formula (I), R¹ is selected from aryl and heteroaryl; wherein said aryl and said heteroaryl are each optionally substituted with one or more groups R^{c}. In still more preferred compounds of formula (I), R¹ is selected from aryl and heteroaryl; wherein said aryl and said heteroaryl are each optionally substituted with one or more groups R^{c}. In still more preferred compounds of formula (I), R¹ is aryl which is optionally substituted with one or more groups R^{c}. In one compound of formula (I), R¹ is aryl which is optionally substituted with one, two or three groups independently selected from —OH, —O—R^{d} and —O—C₁₋₄ alkyl. Still more preferably, R¹ is phenyl, optionally substituted with one, two or three groups independently selected from —OH, —O—R^{d} and —O—C₁₋₄ alkyl.

In other preferred compounds of formula (I), R² is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}, and wherein R² is different from —OH. In more preferred compounds of formula (I), R² is selected from cycloalkyl, heterocycloalkyl, aryl and heteroaryl; wherein said cycloalkyl, said heterocycloalkyl, said aryl and said heteroaryl are each optionally substituted with one or more groups R^{c}. In even more preferred compounds of formula (I), R² is selected from aryl and heteroaryl; wherein said aryl and said heteroaryl are each optionally substituted with one or more groups R^{c}. In still more preferred compounds of formula (I), R² is selected from aryl and heteroaryl; wherein said aryl and said heteroaryl are each optionally substituted with one or more groups R^{c}. Still more preferably, R² is aryl which is optionally substituted with one or more groups R^{c}. In some compounds of formula (I), R² is aryl which is optionally substituted with one, two or three groups independently selected from —OH, —O—R^{d} and —O—C₁₋₄ alkyl. Still more preferably, R² is phenyl, optionally substituted with one, two or three groups independently selected from —OH, —O—R^{d} and —O—C₁₋₄ alkyl.

Alternatively, R¹ and R² are joined together to form, together with the carbon atom(s) that they are attached to, a carbocyclic or heterocyclic ring being optionally substituted with one or more substituents R^{e}; wherein each R^{e} is independently selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.

Preferably, each R^{e} is independently selected from C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, each R^{e} is independently selected from C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, each R^{e} is independently selected from C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, each R^{e} is independently selected from C₁₋₅ alkyl, C₂₋₅ alkenyl, heteroalkyl, heterocycloalkyl, —OR^{b} and —OR^{d}; wherein said alkyl, said alkenyl, said heteroalkyl and said heterocycloalkyl are each optionally substituted with one or more groups independently selected from halogen, —CF₃, —CN —OH and —O—R^{d}. Still more preferably, each R^{e} is independently selected from —OH, —O—C₁₋₅ alkyl, C₁₋₅ alkyl, C₂₋₅ alkenyl, heteroalkyl, heterocycloalkyl and —OR^{d}; wherein said alkyl, said alkenyl, said heteroalkyl, said heterocycloalkyl and the alkyl in said —O—C₁₋₅ alkyl are each optionally substituted with one or more groups independently selected from halogen, —CF₃, —CN —OH and —O—R^{d}. Still more preferably, each R^{e} is independently selected from —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN —OH and —O—R^{d}. Most preferably, each R^{e} is independently selected from —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d}.

R⁴, R⁵ and R⁶ can independently be selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.

Alternatively, R⁴ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and R⁵ and R⁶ are joined together to form, together with the carbon atoms that they are attached to, a carbocyclic or heterocyclic ring being optionally substituted with one or more substituents R^{c}.

In a further alternative, R⁴ and R⁵ are joined together to form, together with the carbon atoms that they are attached to, a carbocyclic or heterocyclic ring being optionally substituted with one or more substituents R^{c}; and R⁶ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}.

R⁴ is preferably selected from hydrogen, C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, R⁴ is selected from hydrogen, C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, R⁴ is selected from hydrogen, C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, R⁴ is selected from hydrogen, C₁₋₅ alkyl, C₂₋₅ alkenyl, heteroalkyl, heterocycloalkyl, —OR^{b} and —OR^{d}; wherein said alkyl, said alkenyl, said heteroalkyl and said heterocycloalkyl are each optionally substituted with one or more groups independently selected from halogen, —CF₃, —CN —OH and —O—R^{d}. Still more preferably, R⁴ is selected from hydrogen, —OH, —O—C₁₋₅ alkyl, C₁₋₅ alkyl, C₂₋₅ alkenyl, heteroalkyl, heterocycloalkyl and —OR^{d}; wherein said alkyl, said alkenyl, said heteroalkyl, said heterocycloalkyl and the alkyl in said —O—C₁₋₅ alkyl are each optionally substituted with one or more groups independently selected from halogen, —CF₃, —CN —OH and —O—R^{d}. Still more preferably, R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN —OH and —O—R^{d}. Most preferably, R⁴ is selected from hydrogen, —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d}.

R⁵ is preferably selected from hydrogen, C₁₋₅ alkyl, C₂₋₅ alkenyl, heteroalkyl, heterocycloalkyl, aryl, heteroaryl, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, R⁵ is selected from hydrogen, C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, R⁵ is selected from hydrogen, C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, R⁵ is selected from hydrogen, C₁₋₅ alkyl, C₂₋₅ alkenyl, —R^{a}—OR^{b} and —R^{a}—OR^{d}; wherein said alkyl and said alkenyl are each optionally substituted with one or more groups R^{c}. Still more preferably, R⁵ is selected from hydrogen, C₁₋₅ alkyl, C₂₋₅ alkenyl, —OR^{b} and —OR^{d}; wherein said alkyl and said alkenyl are each optionally substituted with one or more groups R^{c}. Still more preferably, R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c}; Most preferably, R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};

R⁶ is preferably selected from hydrogen, C₁₋₅ alkyl, C₂₋₅ alkenyl, heteroalkyl, heterocycloalkyl, aryl, heteroaryl, —R^{a}—R^{b}, —R^{a}—OR^{b}, —R^{a}—OR^{d}, —R^{a}—OR^{a}—OR^{b} and —R^{a}—OR^{a}—OR^{d}; wherein said alkyl, said alkenyl, said heteroalkyl, said heterocycloalkyl, said aryl and said heteroaryl are each optionally substituted with one or more groups R^{c}. More preferably, R⁶ is selected from hydrogen, C₁₋₅ alkyl, C₂₋₅ alkenyl, heteroalkyl, heterocycloalkyl, aryl, heteroaryl, —R^{a}—OR^{b} and —R^{a}—OR^{d}; wherein said alkyl, said alkenyl, said heteroalkyl, said heterocycloalkyl, said aryl and said heteroaryl are each optionally substituted with one or more groups R^{c}. Even more preferably, R⁶ is selected from hydrogen, C₁₋₅ alkyl, C₂₋₅ alkenyl, heteroalkyl, heterocycloalkyl, —R^{a}—OR^{b} and —R^{a}—OR^{d}; wherein said alkyl, said alkenyl, said heteroalkyl and said heterocycloalkyl are each optionally substituted with one or more groups R^{c}. Still more preferably, R⁶ is selected from hydrogen, —OH, C₁₋₅ alkyl, C₂₋₅ alkenyl, heterocycloalkyl and —R^{a}—OR^{d}; wherein said alkyl, said alkenyl and said heterocycloalkyl are each optionally substituted with one or more groups R^{c}. Still more preferably, R⁶ is selected from hydrogen, —OH, C₁₋₅ alkyl, C₂₋₅ alkenyl and —R^{a}—OR^{d}; wherein said alkyl and said alkenyl and said heterocycloalkyl are each optionally substituted with one or more groups R^{c}. Still more preferably, R⁶ is selected from hydrogen, —OH, —O—R^{d}, C₁₋₅ alkyl and C₂₋₅ alkenyl, wherein said alkyl and said alkenyl are each optionally substituted with one or more groups R^{c}. Still more preferably, R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —CF₃, —CN —OH and —O—R^{d}. Most preferably, R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —OH and —O—R^{d};

In all compounds of the present invention, each R³ is —O-(rhamnosyl), i.e. the residue to be rhamnosylated by the methods of the present invention, wherein said rhamnosyl is optionally substituted at one or more of its —OH groups with one or more groups independently selected from C₁₋₅ alkyl, C₂₋₅ alkenyl, C₂₋₅ alkynyl, a monosaccharide, a disaccharide and an oligosaccharide. The rhamnosyl group in —O—R³ may be attached to the —O— group via any position. Preferably, the rhamnosyl group is attached to the —O— group via position C1. The optional substituents may be attached to the rhamnosyl group at any of the remaining hydroxyl groups.

In preferred embodiments of the present invention, R³ is —O-α-L-rhamnopyranosyl, —O-α-D-rhamnopyranosyl, —O-β-L-rhamnopyranosyl or —O-β-D-rhamnopyranosyl.

In the present invention, each R^{a} is independently selected from a single bond, C₁₋₅ alkylene, C₂₋₅ alkenylene, arylene and heteroarylene; wherein said alkylene, said alkenylene, said arylene and said heteroarylene are each optionally substituted with one or more groups R^{c}. Preferably, each R^{a} is independently selected from a single bond, C₁₋₅ alkylene and C₂₋₅ alkenylene; wherein said alkylene and said alkenylene are each optionally substituted with one or more groups R^{c}. More preferably, each R^{a} is independently selected from a single bond, C₁₋₅ alkylene and C₂₋₅ alkenylene; wherein said alkylene and said alkenylene are each optionally substituted with one or more groups independently selected from halogen, —CF₃, —CN, —OH and —O—C₁₋₄ alkyl. Even more preferably, each R^{a} is independently selected from a single bond, C₁₋₅ alkylene and C₂₋₅ alkenylene; wherein said alkylene and said alkenylene are each optionally substituted with one or more groups independently selected from —OH and —O—C₁₋₄ alkyl. Still more preferably, each R^{a} is independently selected from a single bond and C₁₋₅ alkylene; wherein said alkylene is optionally substituted with one or more groups independently selected from —OH and —O—C₁₋₄ alkyl. Most preferably, each R^{a} is independently selected from a single bond and C₁₋₅ alkylene.

In the present invention, each R^{b} is independently selected from hydrogen, C₁₋₅ alkyl, C₂₋₅ alkenyl, C₂₋₅ alkynyl, heteroalkyl, cycloalkyl, heterocycloalkyl, aryl and heteroaryl; wherein said alkyl, said alkenyl, said alkynyl, said heteroalkyl, said cycloalkyl, said heterocycloalkyl, said aryl and said heteroaryl are each optionally substituted with one or more groups R^{c}. Preferably, each R^{b} is independently selected from hydrogen, C₁₋₅ alkyl, C₂₋₅ alkenyl, cycloalkyl, heterocycloalkyl, aryl and heteroaryl; wherein said alkyl, said alkenyl, said cycloalkyl, said heterocycloalkyl, said aryl and said heteroaryl are each optionally substituted with one or more groups R^{c}. More preferably, each R^{b} is independently selected from hydrogen, C₁₋₅ alkyl, C₂₋₅ alkenyl, heterocycloalkyl, aryl and heteroaryl; wherein said alkyl, said alkenyl, said heterocycloalkyl, said aryl and said heteroaryl are each optionally substituted with one or more groups R^{c}. Even more preferably, each R^{b} is independently selected from hydrogen, C₁₋₅ alkyl, C₂₋₅ alkenyl, heterocycloalkyl, aryl and heteroaryl; wherein said alkyl, said alkenyl, said heterocycloalkyl, said aryl and said heteroaryl are each optionally substituted with one or more groups R^{c} Still more preferably, each R^{b} is independently selected from hydrogen, C₁₋₅ alkyl, C₂₋₅ alkenyl, heterocycloalkyl, aryl and heteroaryl; wherein said alkyl, said alkenyl, said heterocycloalkyl, said aryl and said heteroaryl are each optionally substituted with one or more groups independently selected from halogen, —CF₃, —CN, —OH and —O—C₁₋₄ alkyl. Still more preferably, each R^{b} is independently selected from hydrogen, C₁₋₅ alkyl, C₂₋₅ alkenyl and aryl; wherein said alkyl, said alkenyl and said aryl are each optionally substituted with one or more groups independently selected from halogen, —CF₃, —CN, —OH and —O—C₁₋₄ alkyl. Still more preferably, each R^{b} is independently selected from hydrogen, C₁₋₅ alkyl and aryl; wherein said alkyl and said aryl are each optionally substituted with one or more groups independently selected from halogen, —CF₃, —CN, —OH and —O—C₁₋₄ alkyl. Still more preferably, each R^{b} is independently selected from hydrogen and C₁₋₅ alkyl; wherein said alkyl is optionally substituted with one or more groups independently selected from halogen, —CF₃, —CN, —OH and —O—C₁₋₄ alkyl. Most preferably, each R^{b} is independently selected from hydrogen and C₁₋₅ alkyl; wherein said alkyl is optionally substituted with one or more groups independently selected from halogen.

In the present invention, each R^{c} is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.

Preferably, each R^{c} is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl) and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.

More preferably, each R^{c} is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl); wherein said alkyl, said alkenyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl.

Even more preferably, each R^{c} is independently selected from C₁₋₅ alkyl, C₂₋₅ alkenyl, —(C₀₋₃ alkylene)-OH and —(C₀₋₃ alkylene)-O—R^{d}; wherein said alkyl, said alkenyl and the alkyl or alkylene moieties comprised in any of the aforementioned groups R^{c} are each optionally substituted with one or more groups independently selected from halogen, —CF₃, —CN, —OH, —O—R^{d} and —O—C₁₋₄ alkyl.

Still more preferably, each R^{c} is independently selected from C₁₋₅ alkyl and C₂₋₅ alkenyl; wherein said alkyl and said alkenyl are each optionally substituted with one or more groups independently selected from halogen, —CF₃, —CN, —OH, —O—R^{d} and —O—C₁₋₄ alkyl.

Still more preferably, each R^{c} is independently selected from C₁₋₅ alkyl and C₂₋₅ alkenyl; wherein said alkyl and said alkenyl are each optionally substituted with one or more groups independently selected from halogen.

In the present invention, each R^{d} is independently selected from a monosaccharide, a disaccharide and an oligosaccharide.

R^{d} may, e.g., be independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

Specific examples of R^{d} include disaccharides such as maltoside, isomaltoside, lactoside, melibioside, nigeroside, rutinoside, neohesperidoside glucose(1→3)rhamnoside, glucose(1→4)rhamnoside, and galactose(1→2)rhamnoside.

Specific examples of R^{d} further include oligosaccharides as maltodextrins (maltotrioside, maltotetraoside, maltopentaoside, maltohexaoside, maltoseptaoside, maltooctaoside), galacto-oligosaccharides, and fructo-oligosaccharides.

In some of the compound of the present invention, each R^{d} is independently selected from arabinosidyl, galactosidyl, galacturonidyl, mannosidyl, glucosidyl, rhamnosidyl, apiosidyl, allosidyl, glucuronidyl, N-acetyl-glucosaminyl, N-acetyl-mannosaminyl, fucosidyl, fucosaminyl, 6-deoxytalosidyl, olivosidyl, rhodinosidyl, and xylosidyl.

The compound of formula (I) may contain at least one OH group in addition to any OH groups in R³, preferably an OH group directly linked to a carbon atom being linked to a neighboring carbon or nitrogen atom via a double bond. Examples of such OH groups include OH groups which are directly attached to aromatic moieties, such as, aryl or heteroaryl groups. One specific example is a phenolic OH group.

Procedures for introducing additional monosaccharides, disaccharides or oligosaccharides at R³, in addition to the rhamnosyl residue, are known in the literature. Examples therefore include the use of cyclodextrin-glucanotranferases (CGTs) and glucansucrases (such as described in EP 1867729 A1) for transfer of glucoside residues at positions C4″-OH and C3″-OH (Shimoda and Hamada 2010, Nutrients 2:171-180, doi:10.3390/nu2020171, Park 2006, Biosci Biotechnol Biochem, 70(4):940-948, Akiyama et al. 2000, Biosci Biotechnol Biochem 64(10): 2246-2249, Kim et al. 2012, Enzyme Microb Technol 50:50-56).

A first preferred example of the compound of formula (I), i.e. a preferred example of a compound to be used as starting material in the methods of the present invention, is a compound of formula (II) or a solvate thereof:

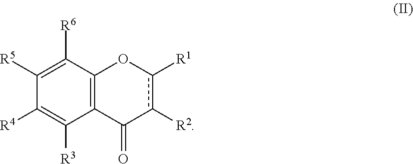

Many examples of the compound of following formula (II) are disclosed herein, such as, compounds of formulae (IIa), (IIb), (IIc) and (IId). It is to be understood that, if reference is made to the compound of formula (II), this reference also includes any of the compounds of formulae (IIa), (IIb), (IIc), (IId), etc.

In formula (II), R¹, R², R³, R⁴, R⁵ and R⁶ are as defined with respect to the compound of general formula (I) including the preferred definitions of each of these residues.

In a first proviso concerning the compound of formula (II), the compounds naringenin-5-O-α-L-rhamnopyranoside and eriodictyol-5-O-α-L-rhamnopyranoside are preferably excluded. In a second proviso, R¹ in the compound of formula (II) is preferably not methyl if R⁴ is hydrogen, R⁵ is —OH and [symbol] is a double bond.

In preferred compounds of formula (II), R¹ is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and R² is selected from hydrogen, C₁₋₅ alkyl and C₂₋₅ alkenyl. In more preferred compounds of formula (II), R¹ is selected from cycloalkyl, heterocycloalkyl, aryl and heteroaryl; wherein said cycloalkyl, said heterocycloalkyl, said aryl and said heteroaryl are each optionally substituted with one or more groups R^{c}; and R² is selected from hydrogen and C₁₋₅ alkyl. In even more preferred compounds of formula (II), R¹ is selected from aryl and heteroaryl; wherein said aryl and said heteroaryl are each optionally substituted with one or more groups R^{c}; and R² is selected from hydrogen and C₁₋₅ alkyl. In still more preferred compounds of formula (II), R¹ is selected from aryl and heteroaryl; wherein said aryl and said heteroaryl are each optionally substituted with one or more groups R^{c}; and R² is selected from hydrogen and C₁₋₅ alkyl. Still more preferably, R¹ is aryl which is optionally substituted with one or more groups R^{c}, and R² is —H. In some compounds of formula (II), R¹ is aryl which is optionally substituted with one, two or three groups independently selected from —OH, —O—R^{d} and —O—C₁₋₄ alkyl, and R² is —H. Still more preferably, R¹ is phenyl, optionally substituted with one, two or three groups independently selected from —OH, —O—R^{d} and —O—C₁₋₄ alkyl; and R² is —H.

In alternatively preferred compounds of formula (II), R² is selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from —OH; and R¹ is selected from hydrogen, C₁₋₅ alkyl and C₂₋₅ alkenyl. In more preferred compounds of formula (II), R² is selected from cycloalkyl, heterocycloalkyl, aryl and heteroaryl; wherein said cycloalkyl, said heterocycloalkyl, said aryl and said heteroaryl are each optionally substituted with one or more groups R^{e}; and R¹ is selected from hydrogen and C₁₋₅ alkyl. In even more preferred compounds of formula (II), R² is selected from aryl and heteroaryl; wherein said aryl and said heteroaryl are each optionally substituted with one or more groups R^{c}; and R¹ is selected from hydrogen and C₁₋₅ alkyl. In still more preferred compounds of formula (II), R² is selected from aryl and heteroaryl; wherein said aryl and said heteroaryl are each optionally substituted with one or more groups R^{c}; and R¹ is selected from hydrogen and C₁₋₅ alkyl. Still more preferably, R² is aryl which is optionally substituted with one or more groups R^{c}, and R¹ is —H. In some of the compounds of formula (II), R² is aryl which is optionally substituted with one, two or three groups independently selected from —OH, —O—R^{d} and —O—C₁₋₄ alkyl, and R¹ is —H. Still more preferably, R² is phenyl, optionally substituted with one, two or three groups independently selected from —OH, —O—R^{d} and —O—C₁₋₄ alkyl; and R¹ is —H.

each R^{c} can preferably independently be selected from halogen, —CF₃, —CN, —OH, —O—R^{d}, —O—C₁₋₄ alkyl, —O-aryl, —S—C₁₋₄ alkyl and —S-aryl.

In preferred compounds of formula (II) each R^{d} is independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

The compound of formula (II) may contain at least one OH group in addition to any OH groups in R³, preferably an OH group directly linked to a carbon atom being linked to a neighboring carbon or nitrogen atom via a double bond. Examples of such OH groups include OH groups which are directly attached to aromatic moieties, such as, aryl or heteroaryl groups. One specific example is a phenolic OH group.

R⁴, R⁵ and R⁶ may each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl).

In some compounds of formula (II), R⁵ is —OH, —O—R^{d} or —O—(C₁₋₅ alkyl). In some compounds of formula (II), R⁴ and/or R⁶ is/are hydrogen or —OH. Most preferably, R² is H or —(C₂₋₅ alkenyl).

Furthermore, R¹ and/or R² may independently be selected from aryl and heteroaryl, wherein said aryl and said heteroaryl are each optionally substituted with one or more groups R^{c}.

A first example of the compound of formula (II) is a compound of the following formula (IIa) or a solvate thereof:

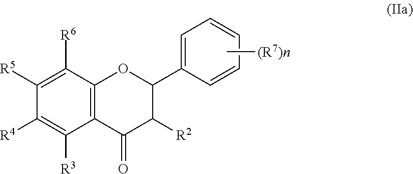

wherein:
R², R³, R⁴, R⁵ and R⁶ are as defined with respect to the compound of general formula (I) including the preferred definitions of each of these residues;
each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl;
n is an integer of 0 to 5, preferably 1, 2, or 3.

Preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl) and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

More preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

Even more preferably, each R⁷ is independently selected from C₁₋₅ alkyl, C₂₋₅ alkenyl, —(C₀₋₃ alkylene)-OH and —(C₀₋₃ alkylene)-O—R^{d}; wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d} and —O—C₁₋₄ alkyl.

The following combination of residues is preferred in compounds of formula (IIa),

R² is selected from hydrogen, C₁₋₅ alkyl, C₂₋₅ alkenyl, and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};
R⁴ is selected from hydrogen, —OH, —O—R^{d}, C₁₋₅ alkyl, C₂₋₅ alkenyl and —O—C₁₋₅ alkyl; wherein said alkyl, said alkenyl and the alkyl in said —O—C₁₋₅ alkyl are each optionally substituted with one or more groups independently selected from halogen, —CF₃, —CN, —OH and —O—R^{d};
R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};
R⁶ is selected from hydrogen, —OH, —O—R^{d}, C₁₋₅ alkyl and C₂₋₅ alkenyl, wherein said alkyl and said alkenyl are each optionally substituted with one or more groups R^{c};
each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl; and
n is an integer of 0 to 3.

The following combination of residues is more preferred in compounds of formula (IIa),

R² is selected from hydrogen, C₁₋₅ alkyl and C₂₋₅ alkenyl, wherein said alkyl and said alkenyl are each optionally substituted with one or more groups independently selected from halogen, —OH and —O—R^{d};
R⁴ is selected from hydrogen, —OH, —O—R^{d}, —O—C₁₋₅ alkyl and C₂₋₅ alkenyl wherein the alkyl in said —O—C₁₋₅ alkyl and said alkenyl are each optionally substituted with one or more groups independently selected from halogen, —OH and —O—R^{d};
R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁶ is selected from hydrogen, —OH, —O—R^{d}, —C₁₋₅ alkyl and C₂₋₅ alkenyl, wherein said alkyl and said alkenyl are each optionally substituted with one or more groups independently selected from halogen, —OH and —O—R^{d};
each R⁷ is independently selected from C₁₋₅ alkyl, C₂₋₅ alkenyl, —(C₀₋₃ alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH, and —O—R^{d}; and
n is 0, 1 or 2.

Even more preferably, the compound of formula (IIa), is selected from the following compounds or solvates thereof:

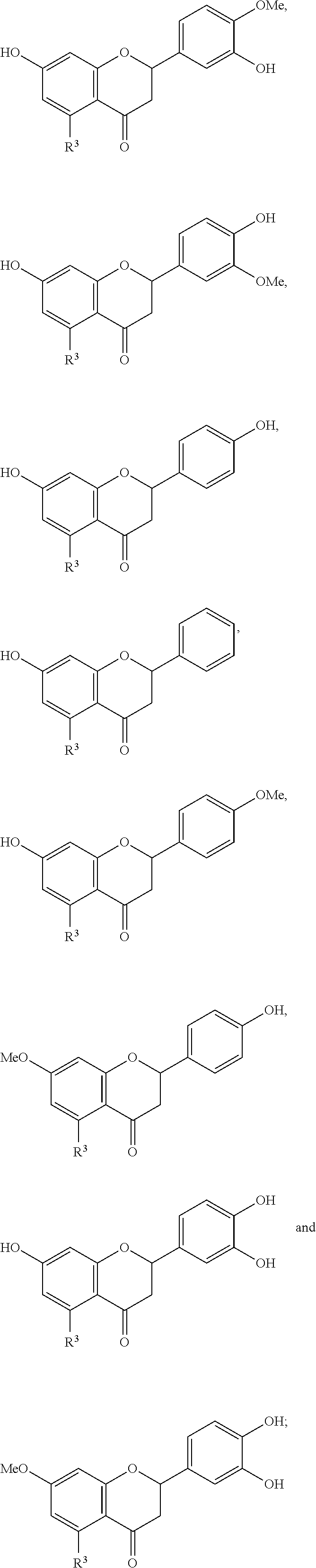

wherein R³ is as defined with respect to the compound of general formula (I).

A second example of the compound of formula (II) is a compound of the following formula (IIb) or a solvate thereof:

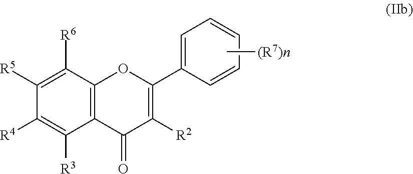

wherein:
R², R³, R⁴, R⁵ and R⁶ are as defined with respect to the compound of general formula (I) including the preferred definitions of each of these residues;
each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl; and
n is an integer of 0 to 5, preferably 1, 2, or 3.

Preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

More preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

Even more preferably, each R⁷ is independently selected from C₁₋₅ alkyl, C₂₋₅ alkenyl, —(C₀₋₃ alkylene)-OH and —(C₀₋₃ alkylene)-O—R^{d}; wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d} and —O—C₁₋₄ alkyl.

The following combination of residues is preferred in compounds of formula (IIb),

R² is selected from hydrogen, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};
R³ is as defined with respect to the compound of general formula (I);
R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};
R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};
R⁶ is selected from hydrogen, —OH, —O—R^{d}, C₁₋₅ alkyl and C₂₋₅ alkenyl; wherein said alkyl and said alkenyl are each optionally substituted with one or more groups R^{c};
each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl; and
n is an integer of 0 to 3.

The following combination of residues is more preferred in compounds of formula (IIb),

R² is selected from hydrogen, C₁₋₅ alkyl and C₂₋₅ alkenyl, wherein said alkyl and said alkenyl are each optionally substituted with one or more groups independently selected from halogen, —OH and —O—R^{d};
R³ is as defined with respect to the compound of general formula (I);
R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁵ is selected from hydrogen, —OH, —O—R^{d}, —O—C₁₋₅ alkyl and C₂₋₅ alkenyl, wherein the alkyl in said —O—C₁₋₅ alkyl and said alkylene are each optionally substituted with one or more groups independently selected from halogen, —OH and —O—R^{d};
R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};
each R⁷ is independently selected from C₁₋₅ alkyl, C₂₋₅ alkenyl, —(C₀₋₃ alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH and —O—R^{d}; and
n is 0, 1 or 2.

Even more preferably, the compound is selected from the following compounds or solvates thereof:

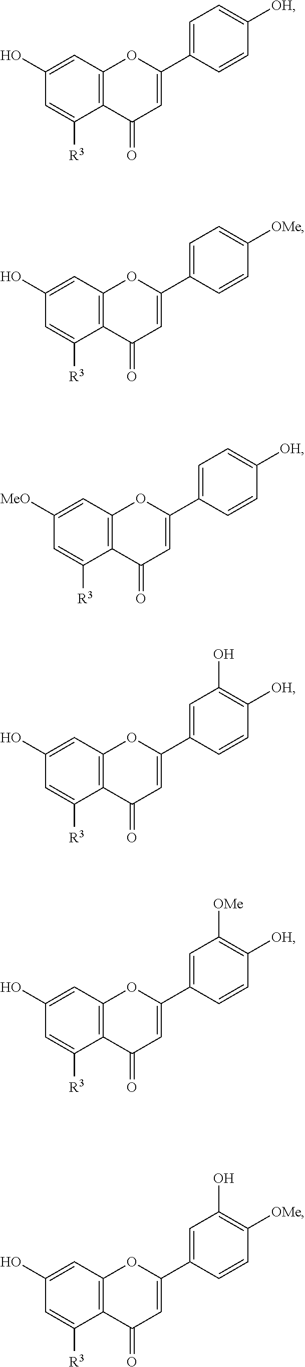

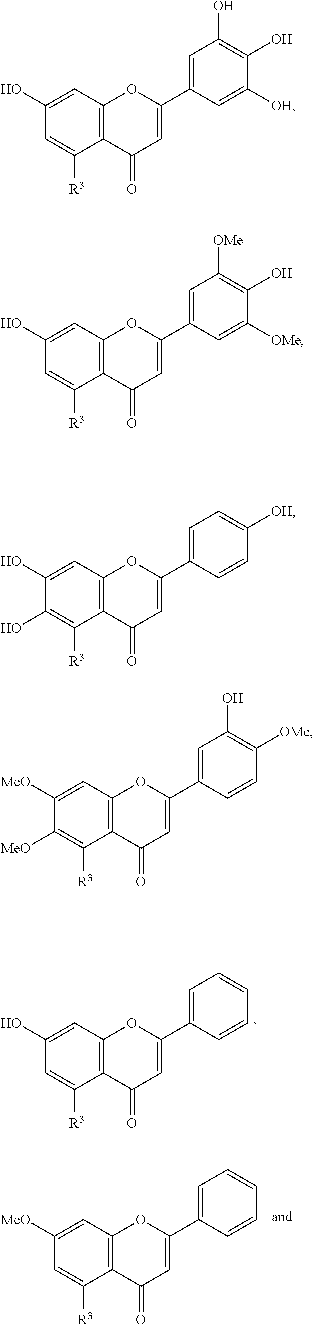

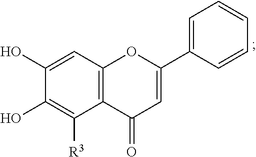

wherein R³ is as defined with respect to the compound of general formula (I).

A third example of the compound of formula (II) is a compound of the following formula (IIc) or a solvate thereof:

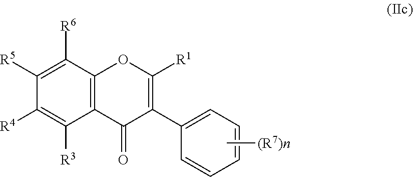

wherein:
R¹, R³, R⁴, R⁵ and R⁶ are as defined with respect to the compound of general formula (I) including the preferred definitions of each of these residues;
each R⁷ is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl, said aryl and said alkylene and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl; and
n is an integer of 0 to 5, preferably 1, 2, or 3.

Preferably, each R⁷ is independently selected from C₁₋₅ alkyl, C₂₋₅ alkenyl, —(C₀₋₃ alkylene)-OH, —(C₀₋₃ alkylene)-O—R^{d}, alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

More preferably, each R⁷ is independently selected from C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl); wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d}, —O—C₁₋₄ alkyl and —S—C₁₋₄ alkyl.

Even more preferably, each R⁷ is independently selected from C₁₋₅ alkyl, C₂₋₅ alkenyl, —(C₀₋₃ alkylene)-OH, —(C₀₋₃ alkylene)-O—R^{d}; wherein said alkyl, said alkenyl and the alkyl or alkylene moieties comprised in any of the aforementioned groups R⁷ are each optionally substituted with one or more groups independently selected from halogen, —CF₃, —CN, —OH, —O—R^{d} and —O—C₁₋₄ alkyl.

The following combination of residues is preferred in compounds of formula (IIc),

R¹ is selected from hydrogen, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN, —OH and —O—R^{d};
R³ is as defined with respect to the compound of general formula (I);
R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN —OH and —O—R^{d};
R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};
R⁶ is selected from hydrogen, —OH, —O—R^{d}, C₁₋₅ alkyl and C₂₋₅ alkenyl, wherein said alkyl and said alkenyl are each optionally substituted with one or more groups R^{c};
each R^{c} is independently selected from C₁₋₅ alkyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl and the alkyl, aryl or alkylene moieties comprised in any of the aforementioned groups R^{c} are each optionally substituted with one or more groups independently selected from halogen, —CF₃, —OH, —O—R^{d} and —O—C₁₋₄ alkyl; and
n is an integer of 0 to 3.

The following combination of residues is more preferred in compounds of formula (IIc),

R¹ is selected from hydrogen, C₁₋₅ alkyl and C₂₋₅ alkenyl, wherein said alkyl and said alkenyl are each optionally substituted with one or more groups independently selected from halogen, —OH and —O—R^{d};
R³ is as defined with respect to the compound of general formula (I);
R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};
each R⁷ is independently selected from C₁₋₅ alkyl, C₂₋₅ alkenyl, alkylene)-OH, —(C₀₋₃ alkylene)-O—R^{d} and —(C₀₋₃ alkylene)-O(C₁₋₅ alkyl); wherein the alkyl, alkenyl and alkylene in the group R⁷ are each optionally substituted with one or more groups independently selected from halogen, —OH and —O—R^{d}; and
n is 0, 1 or 2.

Even more preferred are compounds of formula (IIc), which are is selected from the following compounds or solvates thereof:

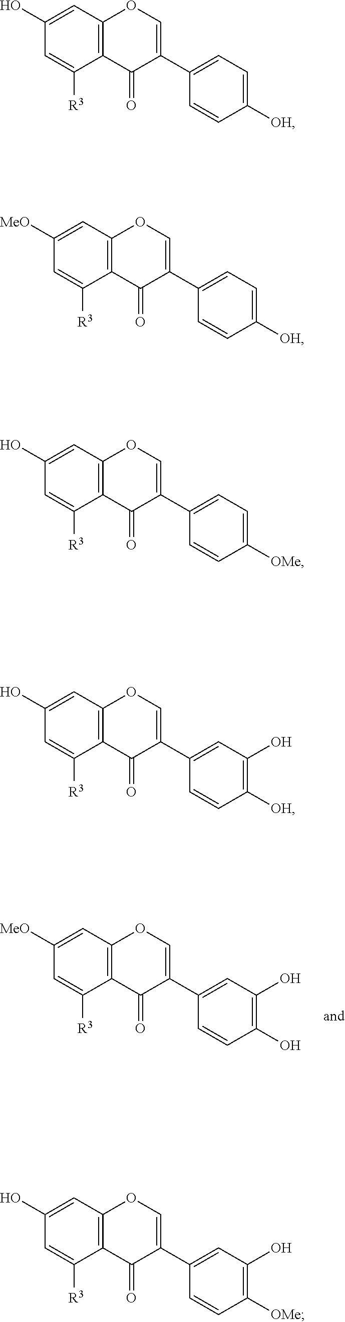

wherein R³ is as defined with respect to the compound of general formula (I).

A fourth example of the compound of formula (II) is a compound of the following formula (IId) or a solvate thereof:

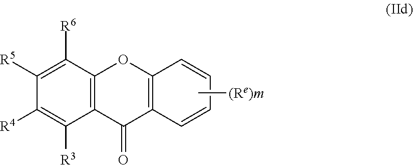

wherein:
R³, R⁴, R⁵, R⁶ and R^{e} are as defined with respect to the compound of general formula (I) including the preferred definitions of each of these residues; and
m is an integer of 0 to 4, preferably 0 to 3, more preferably 1 to 3, even more preferably 1 or 2.

The following combination of residues is preferred in compounds of formula (IId),

R³ is as defined with respect to the compound of general formula (I);
R⁴ is selected from hydrogen, —OH, —O—R^{d}, C₁₋₅ alkyl, C₂₋₅ alkenyl and —O—C₁₋₅ alkyl; wherein said alkyl, said alkenyl, and the alkyl in said —O—C₁₋₅ alkyl are each optionally substituted with one or more groups independently selected from halogen, —CF₃, —CN —OH and —O—R^{d};
R⁵ is selected from hydrogen, —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c};
R⁶ is selected from hydrogen, —OH, —O—R^{d}, C₁₋₅ alkyl and C₂₋₅ alkenyl, wherein said alkyl and said alkenyl are each optionally substituted with one or more groups R^{c};
each R^{e} is independently selected from —OH, —O—R^{d}, C₁₋₅ alkyl, C₂₋₅ alkenyl, —O—C₁₋₅ alkyl and —O-aryl; wherein said alkyl, said alkenyl, the alkyl in said —O—C₁₋₅ alkyl and the aryl in said —O-aryl are each optionally substituted with one or more groups R^{c}; and
m is an integer of 0 to 3.

The following combination of residues is more preferred in compounds of formula (IId),

R³ is as defined with respect to the compound of general formula (I);
R⁴ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁵ is selected from hydrogen, —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d};
R⁶ is selected from hydrogen, —OH, —O—R^{d}, C₁₋₅ alkyl and C₂₋₅ alkenyl, wherein said alkyl and said alkenyl are each optionally substituted with one or more groups independently selected from halogen, —OH and —O—R^{d};
each R^{e} is independently selected from —OH, —O—R^{d}, —O—C₁₋₅ alkyl and C₂₋₅ alkenyl, wherein the alkyl in said —O—C₁₋₅ alkyl and said alkenyl are each optionally substituted with one or more groups independently selected from halogen, —OH and —O—R^{d}; and
m is 0, 1 or 2.

Even more preferred examples of the compound of formula (IId), are compounds selected from the following compounds or solvates thereof:

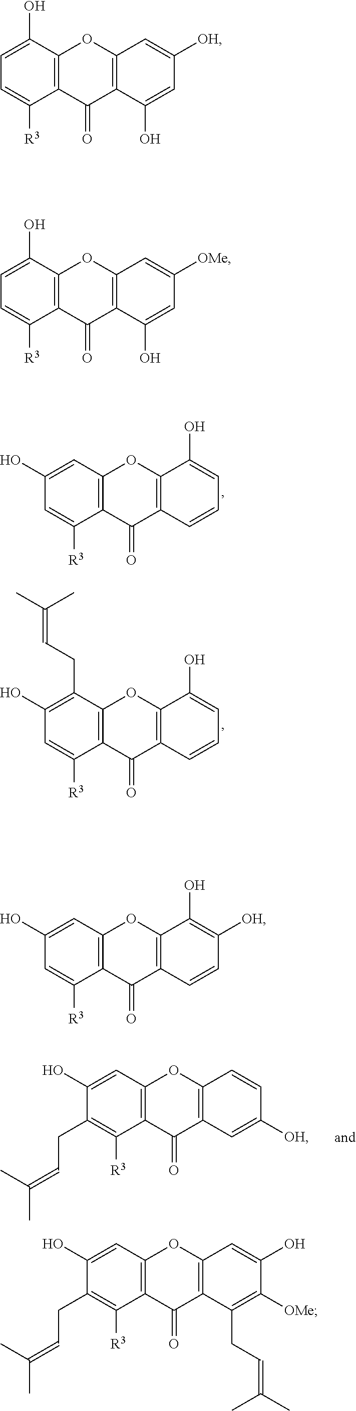

wherein R³ is as defined with respect to the compound of general formula (I).

In preferred compounds of formulae (II), (IIa), (IIb), (IIc) and (IId), R³ is —O-α-L-rhamnopyranosyl, —O-α-D-rhamnopyranosyl, —O-β-L-rhamnopyranosyl or —O-β-D-rhamnopyranosyl.

A second example of a compound of formula (I) is a compound of formula (III) or a solvate thereof:

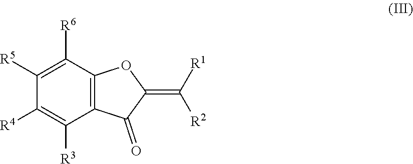

wherein R¹, R², R³, R⁴, R⁵ and R⁶ are as defined with respect to the compound of general formula (I) including the preferred definitions of each of these residues.

In a preferred example of the compounds of formulae (III), R¹ is selected from aryl and heteroaryl, wherein said aryl and said heteroaryl are each optionally substituted with one or more groups R^{c}.

In a preferred example of the compounds of formulae (III), each R^{c} is independently selected from halogen, —CF₃, —CN, —OH, —O—R^{d}, —O—C₁₋₄ alkyl, —O-aryl, —S—C₁₋₄ alkyl and —S-aryl.

In a preferred example of the compounds of formulae (III), the compound contains at least one OH group in addition to any OH groups in R³, preferably an OH group directly linked to a carbon atom being linked to a neighboring carbon or nitrogen atom via a double bond.

In a preferred example of the compounds of formulae (III), R⁴, R⁵ and R⁶ are each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O(C₁₋₅ alkylene)-OH, —(CO_{—3} alkylene)-O(C₁₋₅ alkylene)-O—R^{d} and —(C₀₋₃ alkylene)-O(C₁₋₅ alkylene)-O(C₁₋₅ alkyl).

In a preferred example of the compounds of formulae (III), R⁵ is —OH, —O—R^{d} or —O—(C₁₋₅ alkyl).

In a preferred example of the compounds of formulae (III), R⁴ and/or R⁶ is/are hydrogen or —OH.

Particular examples of the compound of formula (III) include the following compounds or solvates thereof:

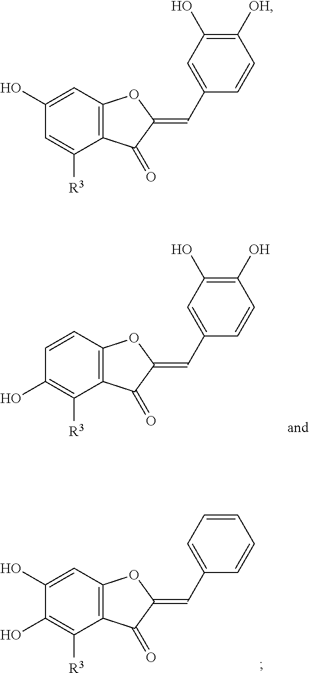

wherein R³ is as defined with respect to the compound of general formula (I).

In a preferred example of the compounds of formula (III), R³ is —O-α-L-rhamnopyranosyl, —O-α-D-rhamnopyranosyl, —O-β-L-rhamnopyranosyl or —O-β-D-rhamnopyranosyl.

In a preferred example of the compounds of formula (III), each R^{d} is independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

Yet a further example of a compound of formula (I) is a compound of formula (IV) or a solvate thereof:

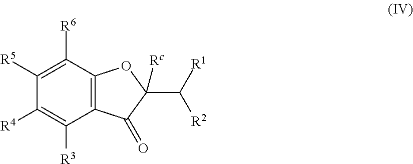

wherein R¹, R², R³, R⁴, R⁵, R⁶ and R^{e} are as defined with respect to the compound of general formula (I) including the preferred definitions of each of these residues.

In a preferred example of the compounds of formula (IV), R¹ is selected from aryl and heteroaryl, wherein said aryl and said heteroaryl are each optionally substituted with one or more groups R^{c}.

In a preferred example of the compounds of formula (IV), each R^{c} is independently selected from halogen, —CF₃, —CN, —OH, —O—R^{d}, —O—C₁₋₄ alkyl, —O-aryl, —S—C₁₋₄ alkyl and —S-aryl.

In a preferred example of the compounds of formula (IV), the compound contains at least one OH group in addition to any OH groups in R³, preferably an OH group directly linked to a carbon atom being linked to a neighboring carbon or nitrogen atom via a double bond.

In a preferred example of the compounds of formula (IV), R⁴, R⁵ and R⁶ are each independently selected from hydrogen, C₁₋₅ alkyl, C₂₋₅ alkenyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O—R^{d} and —(C₀₋₃ alkylene)-O(C₁₋₅ alkylene)-O(C₁₋₅ alkyl).

In a preferred example of the compounds of formula (IV), R⁵ is —OH, —O—R^{d} or —O—(C₁₋₅ alkyl).

In a preferred example of the compounds of formula (IV), R⁴ and/or R⁶ is/are hydrogen or —OH.

Particular examples of the compound of formula (IV) include the following compounds or solvates thereof:

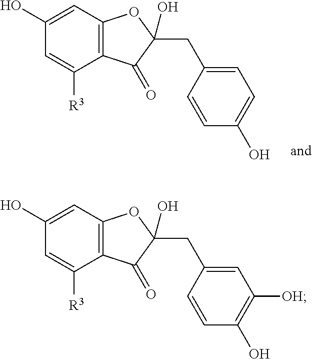

wherein R³ is as defined with respect to the compound of general formula (I).

In a preferred example of the compounds of formula (IV), R³ is —O-α-L-rhamnopyranosyl, —O-α-D-rhamnopyranosyl, —O-β-L-rhamnopyranosyl or —O-β-D-rhamnopyranosyl.

In a preferred example of the compounds of formula (IV), each R^{d} is independently selected from arabinosidyl, galactosidyl, galacturonidyl, mannosidyl, glucosidyl, rhamnosidyl, apiosidyl, allosidyl, glucuronidyl, N-acetyl-glucosamidyl, N-acetyl-mannosidyl, fucosidyl, fucosaminyl, 6-deoxytalosidyl, olivosidyl, rhodinosidyl, and xylosidyl.

The present invention is further described by reference to the following non-limiting figures and examples.

The Figures show:

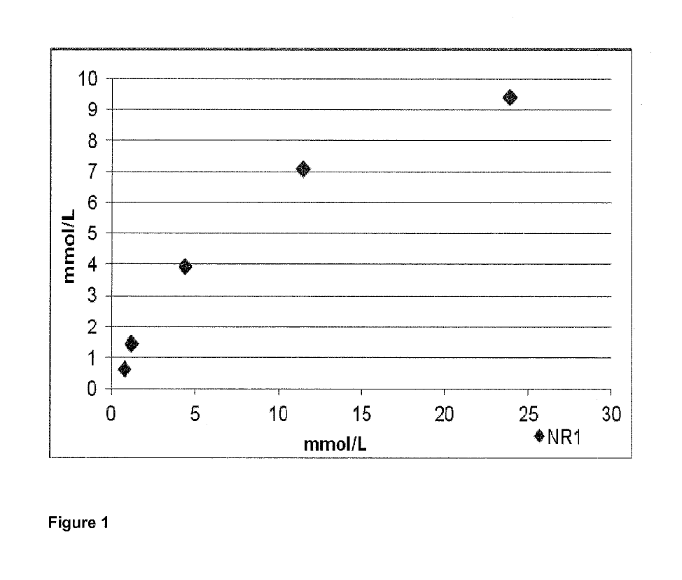

FIG. 1: Determination of solubility of naringenin-5-O-α-L-rhamnoside (NR1) in water. Defined concentrations of NR1 were 0.22 μm-filtered before injection to HPLC. Soluble concentrations were calculated from peak areas by determined regression curves.

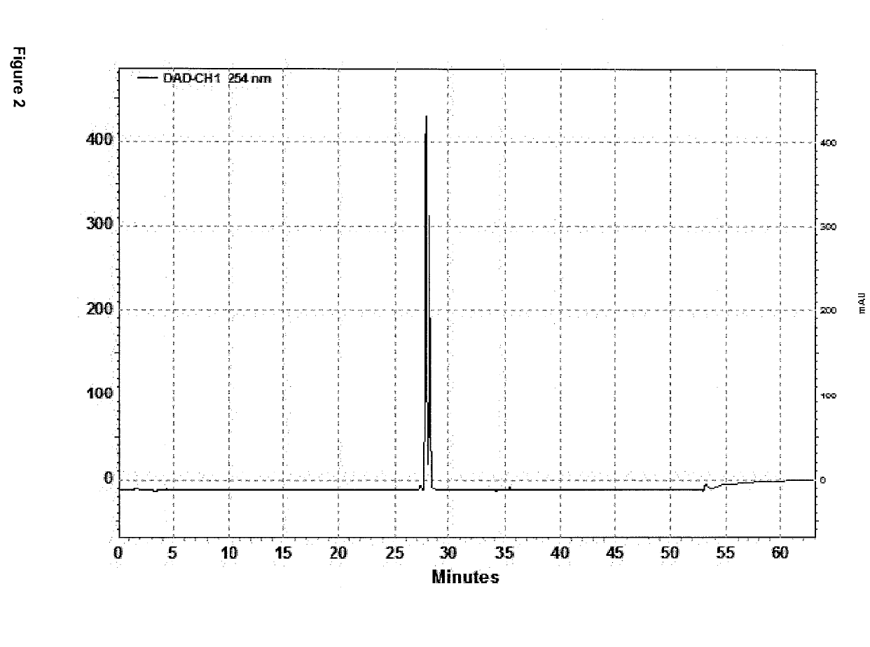

FIG. 2: HPLC-chromatogram of naringenin-5-O-α-L-rhamnoside

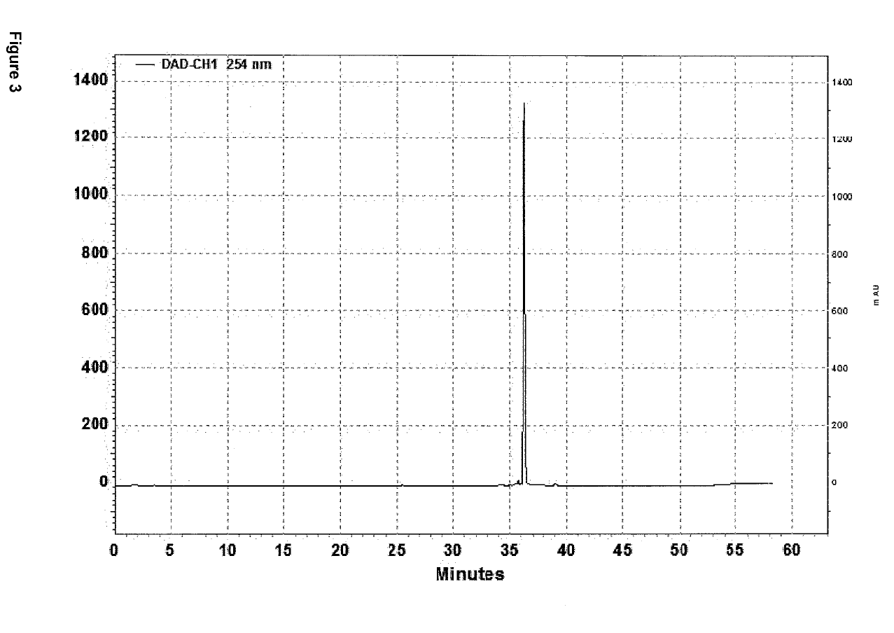

FIG. 3: HPLC-chromatogram of naringenin-4′-O-α-L-rhamnoside

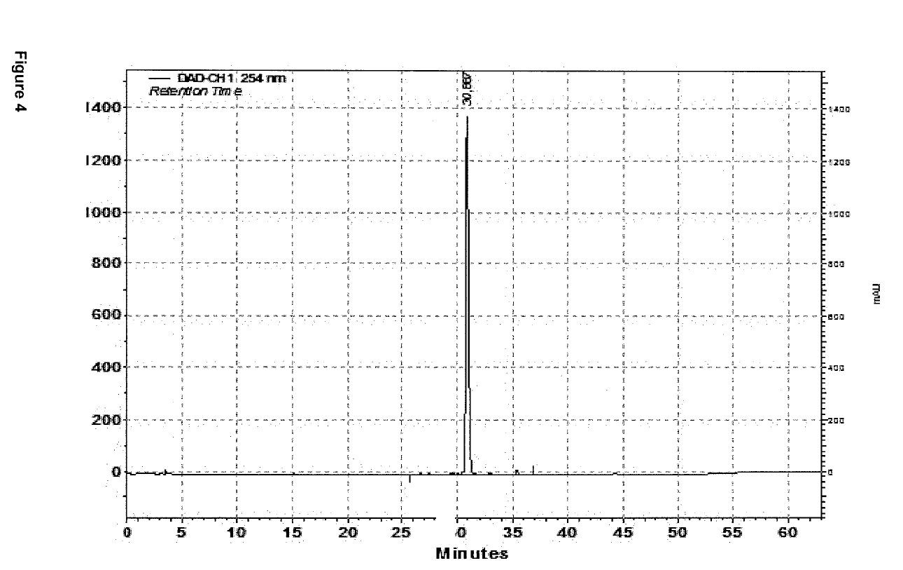

FIG. 4: HPLC-chromatogram of prunin (naringenin-7-O-β-D-glucoside)

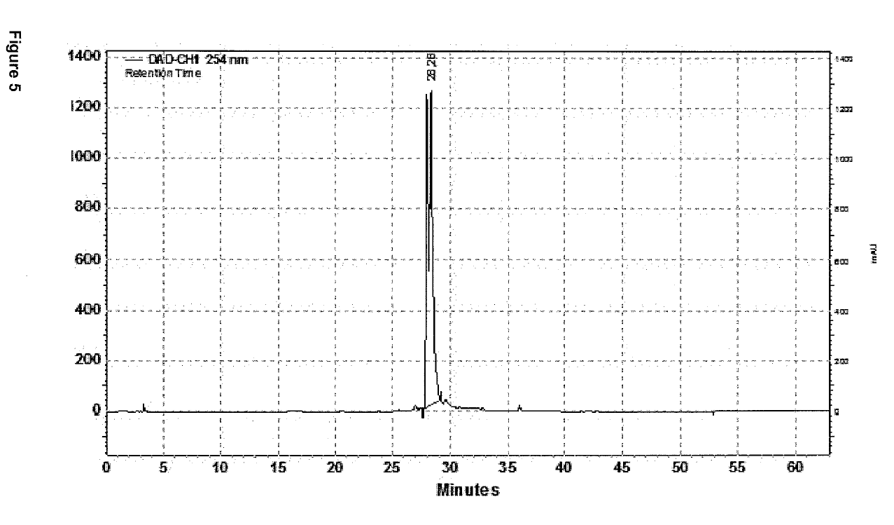

FIG. 5: HPLC-chromatogram of homoeriodictyol-5-O-α-L-rhamnoside (HEDR1)

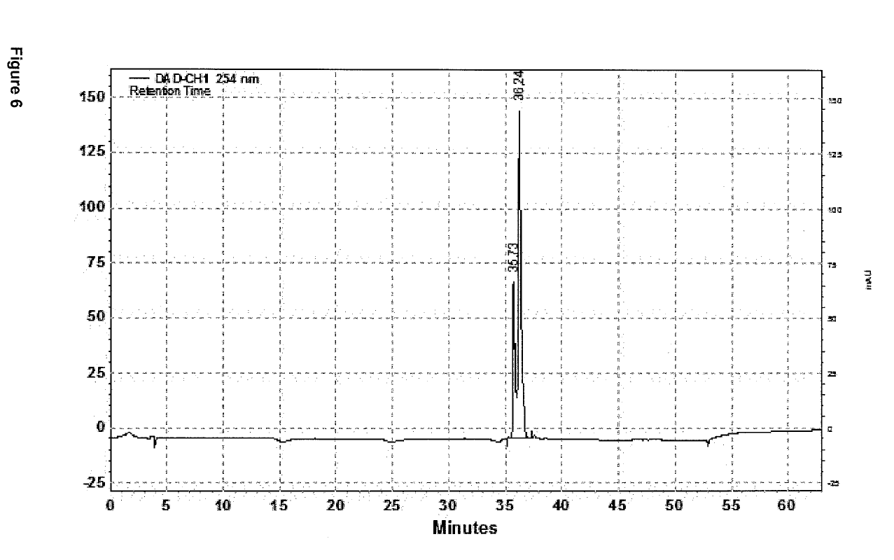

FIG. 6: HPLC-chromatogram of HEDR3 (4:1 molar ratio of homoeriodictyol-7-O-α-L-rhamnoside and homoeriodictyol-4′-O-α-L-rhamnoside)

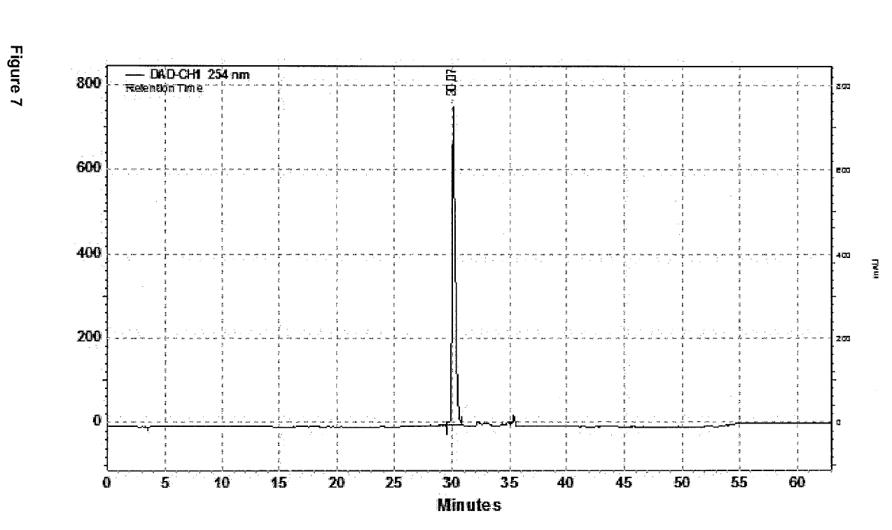

FIG. 7: HPLC-chromatogram of homoeriodictyol-4′-O-β-D-glucoside (HED4′Glc)

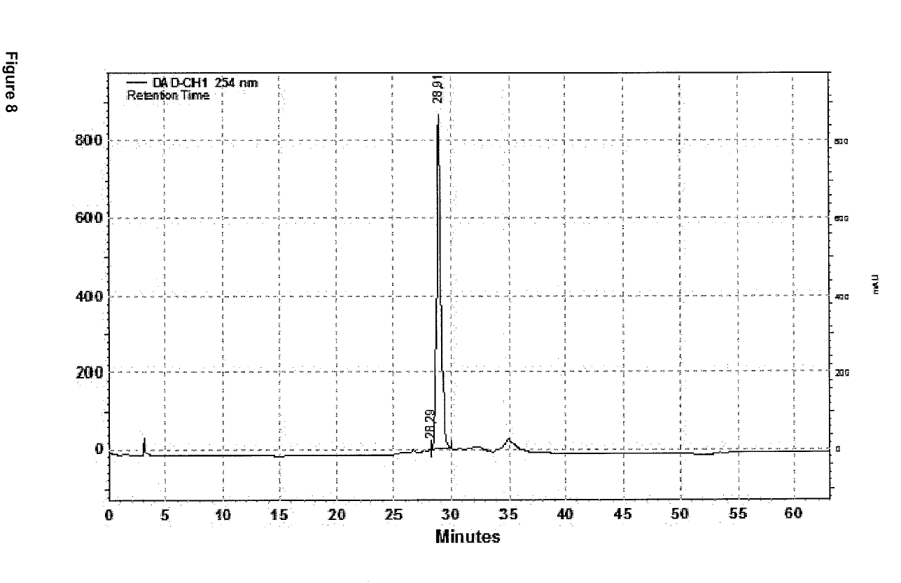

FIG. 8: HPLC-chromatogram of hesperetin-5-O-α-L-rhamnoside (HESR1)

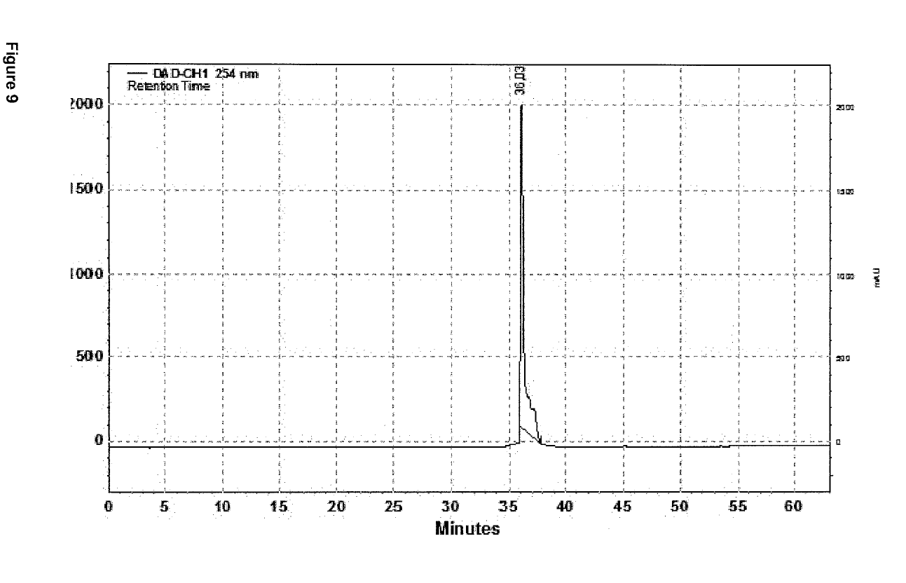

FIG. 9: HPLC-chromatogram of hesperetin-3′-O-α-L-rhamnoside (HESR2)

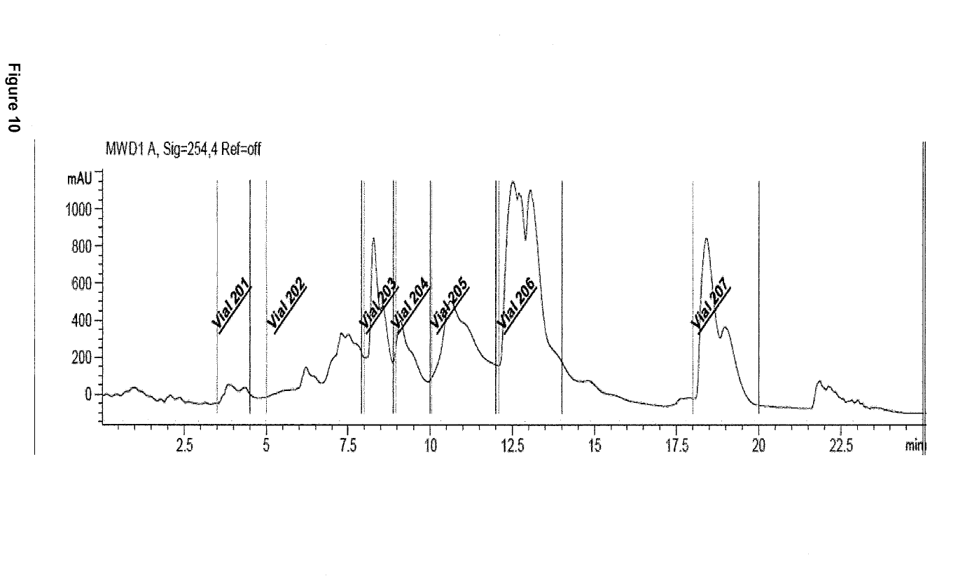

FIG. 10: UV₂₅₄-chromatogram of hesperetin bioconversion 141020, sample injection volume was 1.2 L applied by the pumping system

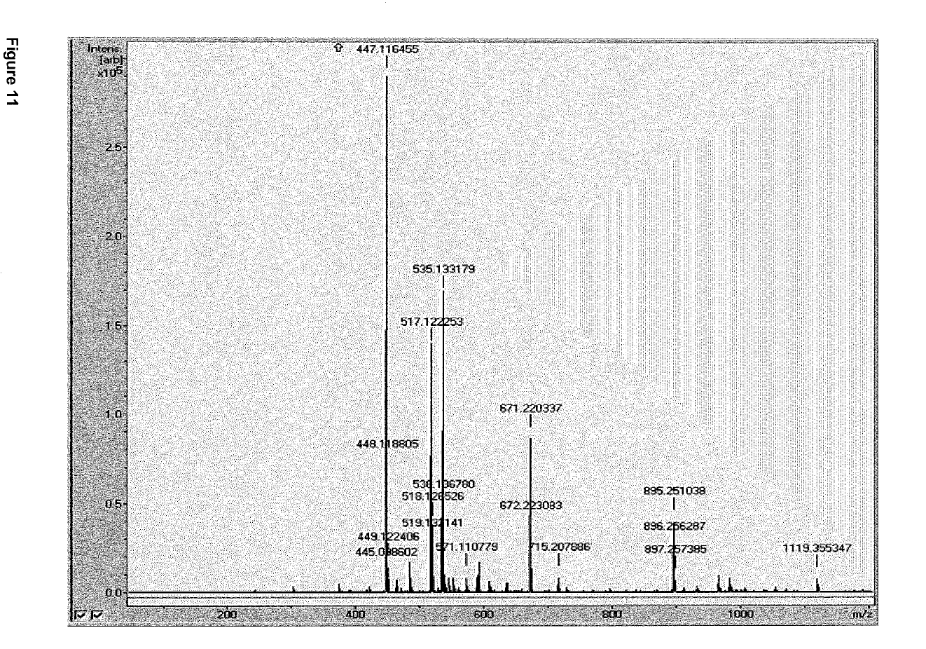

FIG. 11: ESI-TOF negative mode MS-analysis of fraction 3 from hesperetin bioconversion_141020

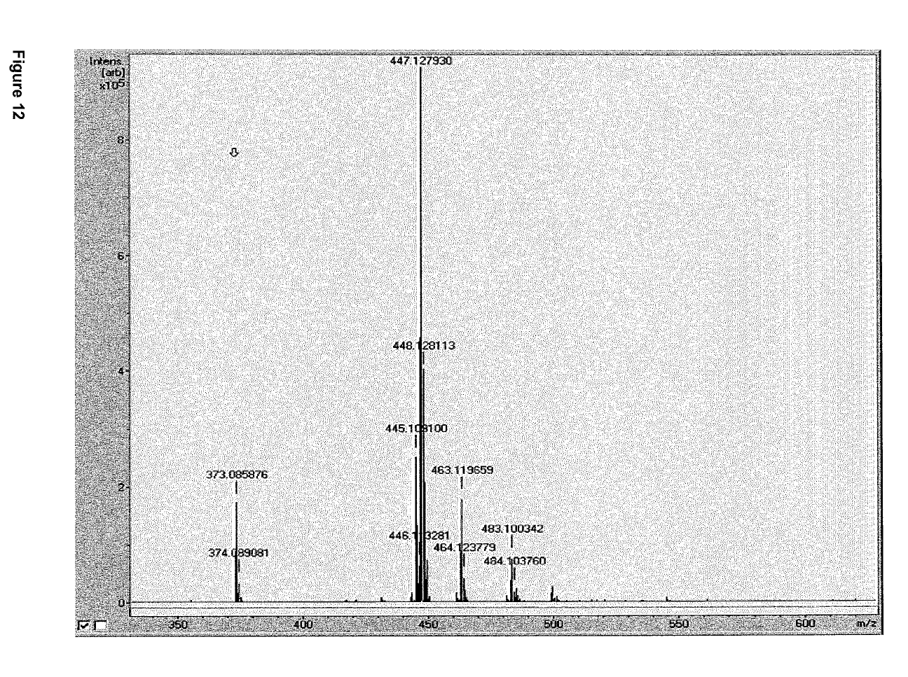

FIG. 12: ESI-TOF negative mode MS-analysis of fraction 6 from hesperetin bioconversion_141020

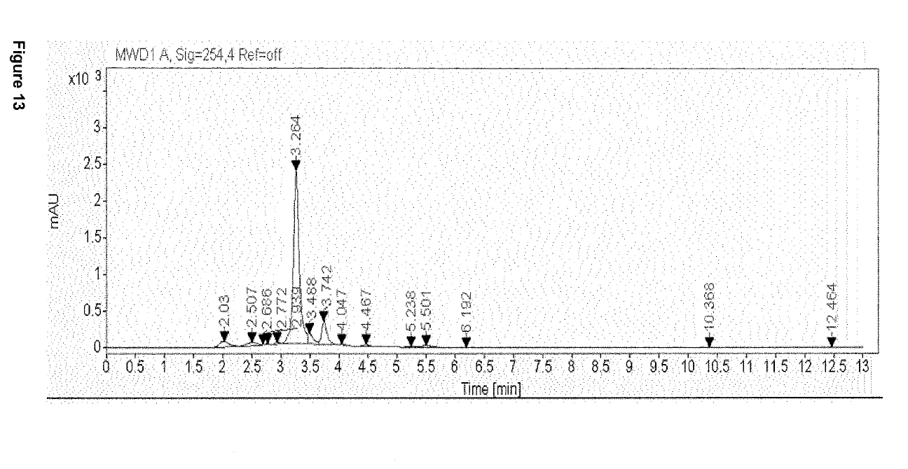

FIG. 13: prepLC UV₂₅₄-chromatogram of PFP-HPLC of fraction 3 bioconversion_141020; the main peak (HESR1) between 3.1 min and 3.5 min was HESR1.

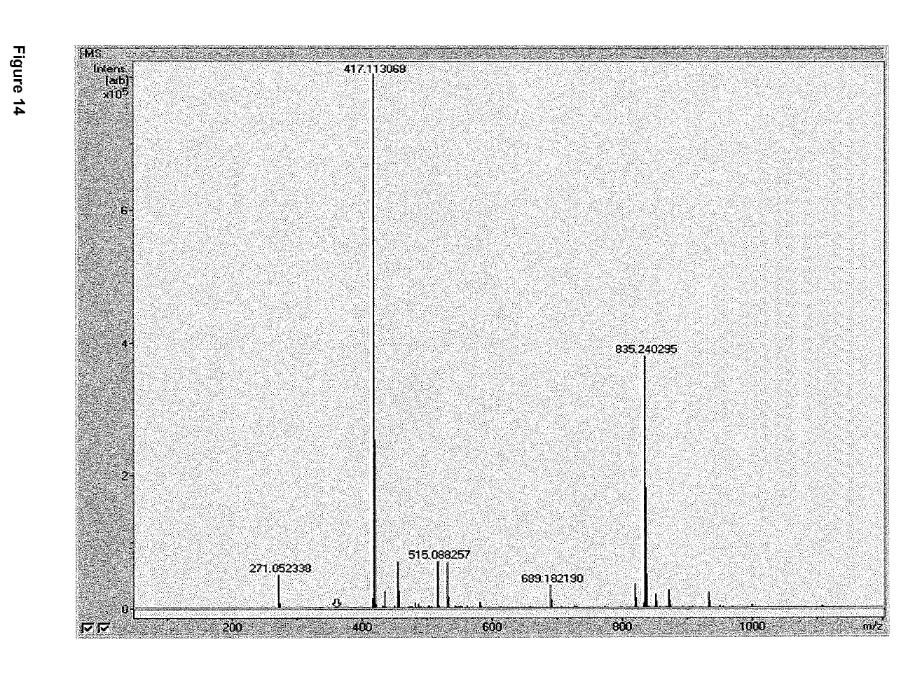

FIG. 14: ESI-TOF negative mode MS-analysis of fraction 3 from 140424_Naringenin-PetC

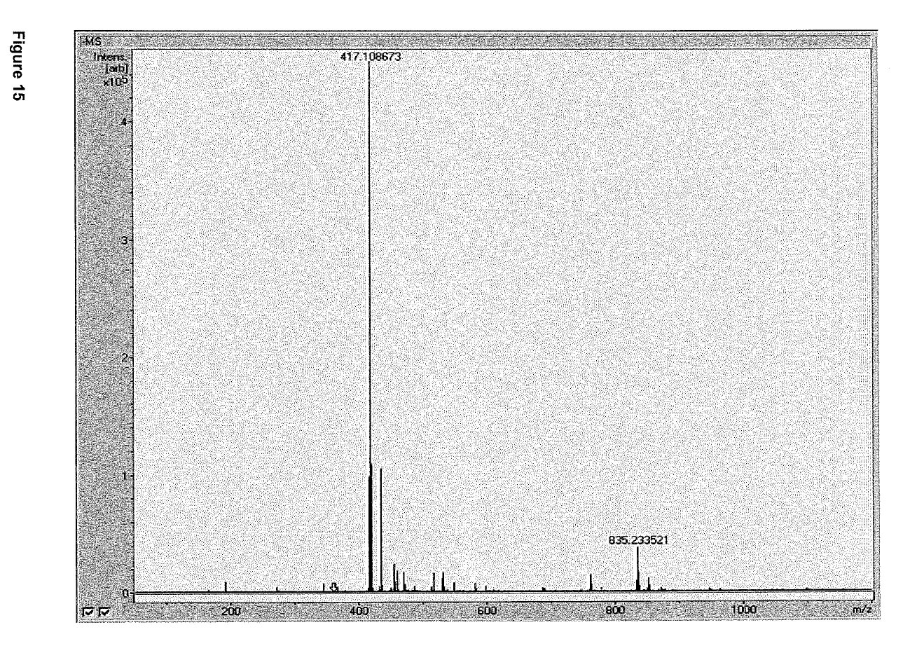

FIG. 15: ESI-TOF negative mode MS-analysis of fraction 5 from 140424_Naringenin-PetC

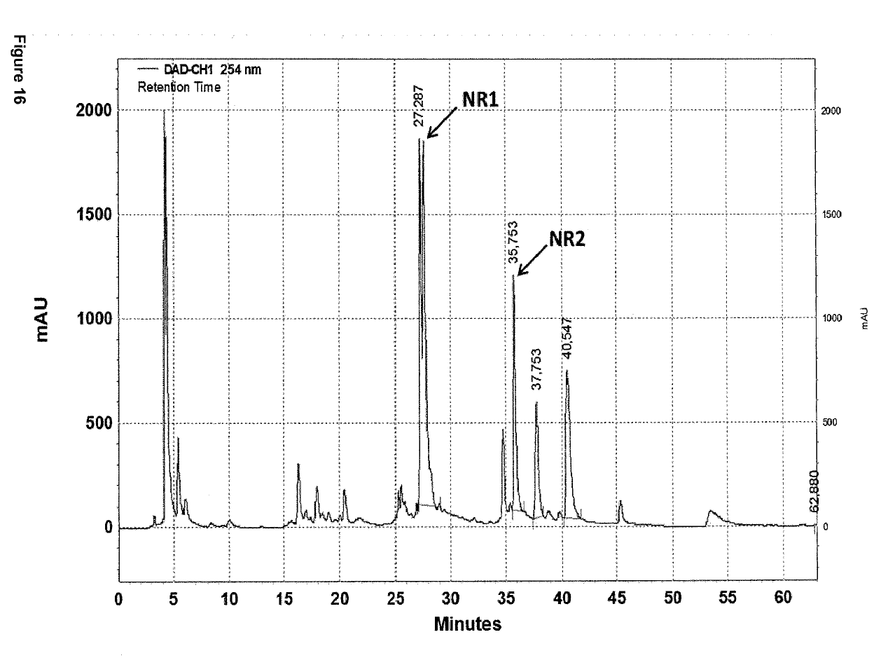

FIG. 16: UV-chromatogram of conversion after 24 h in bioreactor unit 1 150603_Naringenin-PetC

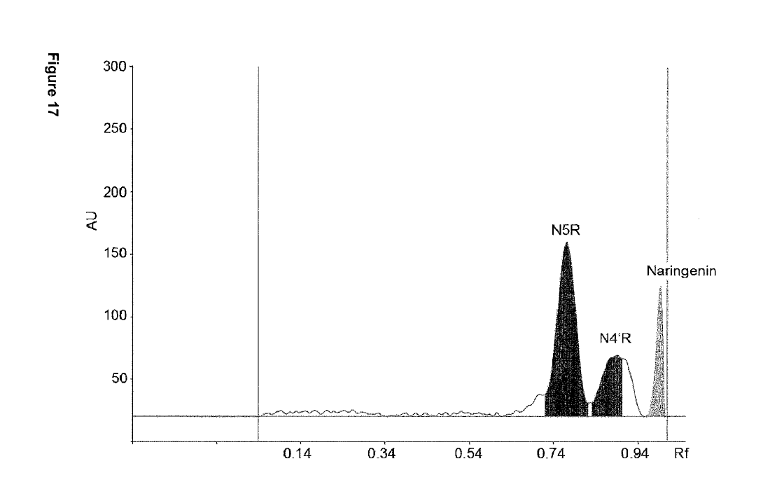

FIG. 17: UV₃₃₀ chromatogram of an extract from a naringenin biotransformation with PetD

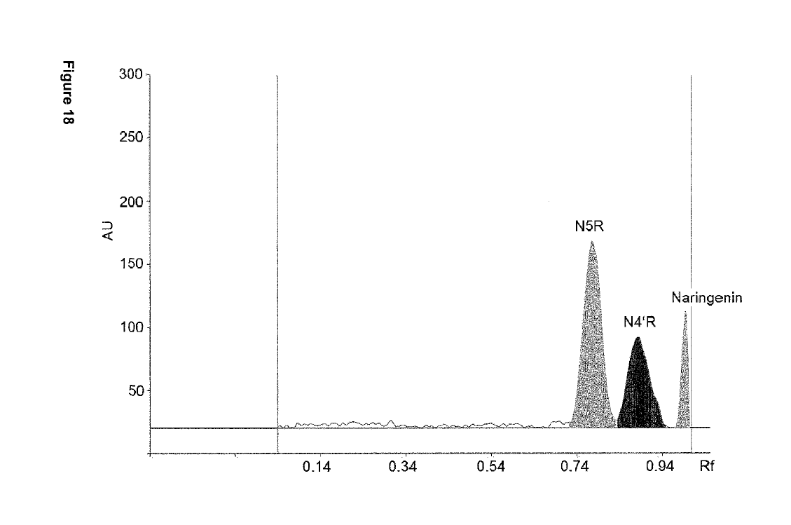

FIG. 18: UV₃₃₀ chromatogram of an extract from a naringenin biotransformation with PetC

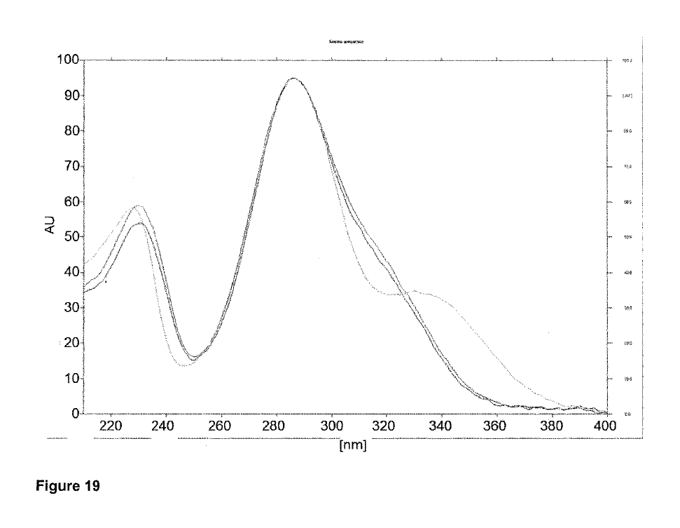

FIG. 19: UV 210-400 nm absorbance spectra of N5R peaks from figures U1 (middle) and U2 (dark) vs. prunin, the naringenin-7-O-β-D-glucoside (light).

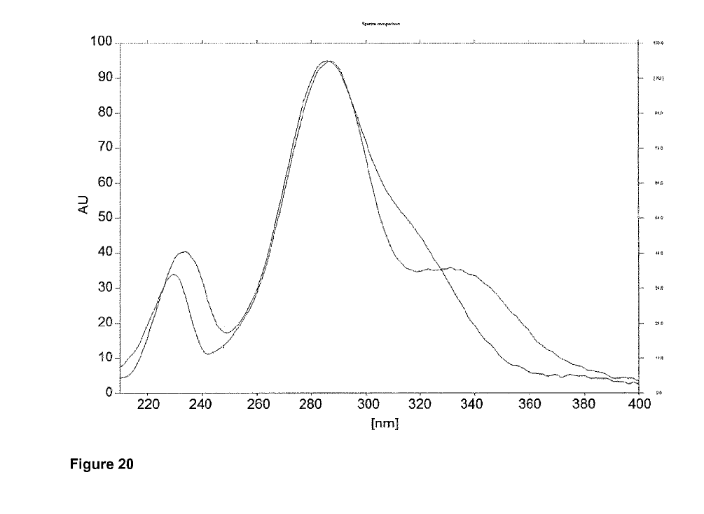

FIG. 20: UV 210-400 nm absorbance spectra of GTF product peak Rf 0.77 (dark) vs. prunin (light).

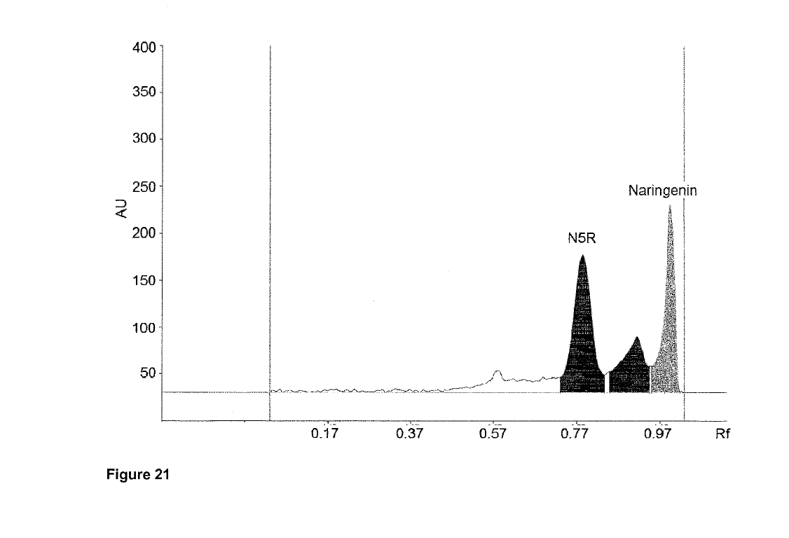

FIG. 21: UV₃₃₀ chromatogram of an extract from a naringenin biotransformation with PetF

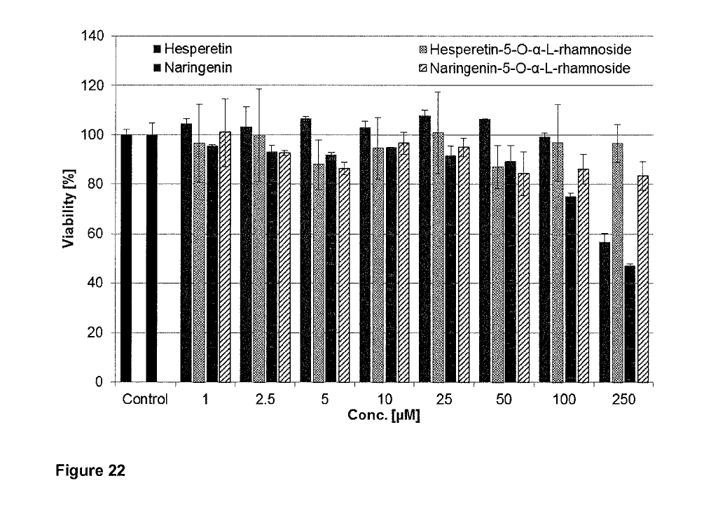

FIG. 22: Cytotoxicity of flavonoid-5-O-α-L-rhamnosides on normal human epidermal keratinocytes

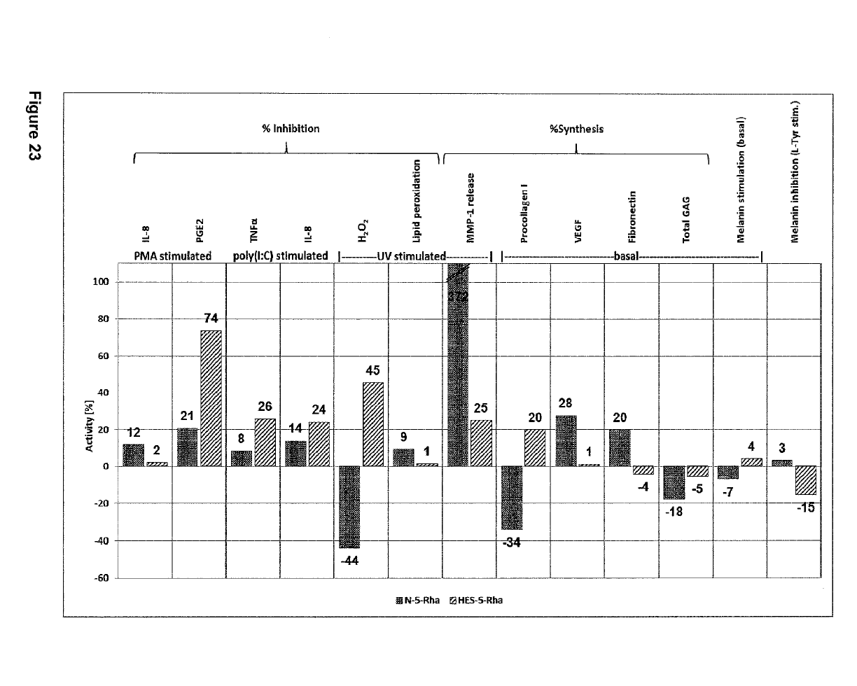

FIG. 23: antiinflammatory, protecting, and stimulating activities of flavonoid-5-O-α-L-rhamnosides on normal human epidermal keratinocytes, normal human dermal fibroblasts, and normal human epidermal melanocytes

### EXAMPLES

The compounds described in this section are defined by their chemical formulae and their corresponding chemical names. In case of conflict between any chemical formula and the corresponding chemical name indicated herein, the present invention relates to both the compound defined by the chemical formula and the compound defined by the chemical name

### Part A: Preparation of 5-O-Rhamnosylated Flavonoids

### Example A1—Preparation of Media and Buffers

The methods of the present invention can be used to produce rhamnosylated flavonoids, as will be shown in the appended Examples.

Several growth and biotransformation media were used for the rhmanoslyation of flavonoids. Suitable media thus include: Rich Medium (RM) (Bacto peptone (Difco) 10 g, Yeast extract 5 g, Casamino acids (Difco) 5 g, Meat extract (Difco) 2 g, Malt extract (Difco) 5 g, Glycerol 2 g, MgSO₄×7 H₂O 1 g, Tween 80 0.05 g and H₂O ad 1000 mL at a final pH of about 7.2); Mineral Salt Medium (MSM) (Buffer and mineral salt stock solution were autoclaved. After the solutions had cooled down, 100 mL of each stock solution were joined and 1 mL vitamin and 1 mL trace element stock solution were added. Then sterile water was added to a final volume of 1 L. The stock solutions were: Buffer stock solution (10×) of Na₂HPO₄ 70 g, KH₂PO₄ 20 g and H₂O ad 1000 mL; Mineral salt stock solution (10×) of (NH₄)₂SO₄ 10 g, MgCl₂×6 H₂O 2 g, Ca(NO₃)₂×4 H₂O 1 g and H₂O ad 1000 mL; Trace element stock solution (1000×) of EDTA 500 mg, FeSO₄×7 H₂O 300 mg, CoCl₂×6 H₂O 5 mg, ZnSO₄×7 H₂O 5 mg, MnCl₂×4 H₂O 3 mg, NaMoO₄×2 H₂O 3 mg, NiCl₂×6 H₂O 2 mg, H₃BO₃ 2 mg, CuCl₂×2 H₂O 1 mg and H₂O ad 200 mL. The solution was sterile filtered. Vitamin stock solution (1000×) of Ca-Pantothenate 10 mg, Cyanocobalamine 10 mg, Nicotinic acid 10 mg, Pyridoxal-HCl 10 mg, Riboflavin 10 mg, Thiamin-HCl 10 mg, Biotin 1 mg, Folic acid 1 mg, p-Amino benzoic acid 1 mg and H₂O ad 100 mL. The solution was sterile filtered); Lysogeny Broth (LB) (Yeast extract 5 g, Peptone 10 g, NaCl 5 g and H₂O ad 1000 mL); Terrific Broth (TB) (casein 12 g, yeast extract 24 g, K₂HPO₄ 12.5 g, KH₂PO₄ 2.3 g and H₂O ad 1000 mL at pH 7.2). In some experiments, in particular when the concentration of dissolved oxygen (DO) was above about 50%, nutrients were added to the solution. This was done using a feed solution of Glucose 500 g, MgSO₄ 10 g, thiamine 1 mg and H₂O ad 1000 mL. In some experiments, in particular when cells expressing glycosyl transferase were harvested prior to starting the production of rhamnosylated flavonoids, cells were resuspended in a buffer solution, in particular phosphate buffer saline (PBS). The solution was prepared using NaCl 150 mM, K₂HPO₄/KH₂PO₄ 100 mM at a pH of 6.4 to 7.4.

### Example A2 Glycosyl Transferases Used for the Production of Rhamnosylated Flavonoids

Several different glycosyl transferases were used in the methods of the present invention to produce rhamnosylated flavonoids. In particular, the glycosyltransferases (GTs) used for flavonoid rhamnoside production were
1. GTC, a GT derived metagenomically (AGH18139), preferably having an amino acid sequence as shown in SEQ ID NO:3, encoded by a polynucleotide as shown in SEQ ID NO:4. A codon-optimized sequence for expression in E. coli is shown in SEQ ID NO:27.
2. GTD, a GT from Dyadobacter fermentans (WP_015811417), preferably having an amino acid sequence as shown in SEQ ID NO:5, encoded by a polynucleotide as shown in SEQ ID NO:6. A codon-optimized sequence for expression in E. coli is shown in SEQ ID NO:28.
3. GTF, a GT from Fibrisoma limi (WP_009280674), preferably having an amino acid sequence as shown in SEQ ID NO:7, encoded by a polynucleotide as shown in SEQ ID NO:8. A codon-optimized sequence for expression in E. coli is shown in SEQ ID NO:29.
4. GTS from Segetibacter koreensis (WP_018611930) preferably having an amino acid sequence as shown in SEQ ID NO:9, encoded by a polynucleotide as shown in SEQ ID NO:10. A codon-optimized sequence for expression in E. coli is shown in SEQ ID NO:30.
5. Chimera 3 with AAs 1 to 316 of GTD and AAs 324 to 459 of GTC preferably having an amino acid sequence as shown in SEQ ID NO: 58, encoded by a polynucleotide as shown in SEQ ID NO: 59. A codon-optimized sequence for expression in E. coli is shown in SEQ ID NO: 60.
6. Chimera 4 with AAs 1 to 268 of GTD and AAs 276 to 459 of GTC preferably having an amino acid sequence as shown in SEQ ID NO: 61, encoded by a polynucleotide as shown in SEQ ID NO: 62. A codon-optimized sequence for expression in E. coli is shown in SEQ ID NO: 63.
7. Chimera 1 frameshift with AAs 1 to 234 of GTD and AAs 242 to 443 of GTC preferably having an amino acid sequence as shown in SEQ ID NO: 56, encoded by a polynucleotide as shown in SEQ ID NO: 57.

The GT genes were amplified by PCR using respective primers given in Table A1. Purified PCR products were ligated into TA-cloning vector pDrive (Qiagen, Germany) Chemically competent E. coli DH5α were transformed with ligation reactions by heat shock and positive clones verified by blue/white screening after incubation. GT from Segetibacter koreensis was directly used as codon-optimized nucleotide sequence.

Chimera 3 and chimera 4 were created from the codon-optimized nucleotide sequences from GTD and GTC, while chimera 1 was constructed from the SEQ ID NO:4 and SEQ ID NO:6. Chimera 1 was created according to the ligase cycling reaction method described by Kok (2014) ACS Synth Biol 3(2):97-106. Thus, the two nucleotide sequences of each chimeric fragment were amplified via PCR and were assembled using a single-stranded bridging oligo which is complementary to the ends of neighboring nucleotide parts of both fragments. A thermostable ligase was used to join the nucleotides to generate the full-length sequence of the chimeric enzyme.

Chimera 3 and chimera 4 were constructed according to the AQUA cloning method described by Beyer (2015) PLoS ONE 10(9):e0137652. Therefore, the nucleotide fragments were amplified with complementary regions of 20 to 25 nucleotides, agarose-gel purified, mixed in water, incubated for 1 hour at room temperature and transformed into chemically competent E. coli DH5α. The primers used for the chimera construction are listed in Table A2.

TABLE A1
Primers used for the amplification of the GT genes by PCR

| Enzyme | Primer name | Sequence (5′→3′) |
|---|---|---|
| GTC | GTC-NdeI-for | CATATGAGTAATTTATTTTCTTCACAAAC |
| | GTC-BamHI-rev | GGATCCTTAGTATATCTTTTCTTCTTC |
| GTD | GTF_XhoI_for | CTCGAGATGACGAAATACAAAAATGAAT |
| | GTF_BamHI_rev | GGATCCTTAACCGCAAACAACCCGC |
| GTF | GTL_XhoI_for | CTCGAGATGACAACTAAAAAAATCCTGTT |
| | GTL_BamHI_rev | GGATCCTTAGATTGCTTCTACGGCTT |
| GTS | GTSopt_pET_fw | GGGAATTCCATATGATGAAATATATCAGCTCCATTCAG |
| | GTSopt_pET_rv | CGGGATCCTTAAACCAGAACTTCGGCCTGATAG |

TABLE A2
Primers used for the construction of chimeric enzymes

| Enzyme | Primer name | Sequence (5′→3′) |
|---|---|---|
| Chimera 1 | Bridge_P1_pETGTD | GCGGCCATATCGACGACGACGACAAGCATATGACGAAATAC |
| | | AAAAATGAATTAACAGGT |
| | Bridge_P1_GTCpET | GGAAGAAGAAAAGATATACTAAGGATCCGGCTGCT |
| | | AACAAAGCCCGAAAGG |
| | Chim_P1_D_Nde_for | CATATGACGAAATACAAAAATGAATT |
| | Chim_P1_D_rev | GCGGTCATACTCAAATGATT |
| | Chim_P1_C_for | AGTGATCTGGGAAAAAATATC |
| | Chim_P1_C_Bam_rev | GGATCCTTAGTATATCTTTTCTTCTTCCT |
| Chimera 3 | GTDopt_pEt_fw | GGGAATTCCATATGATGACCAAATACAAAAATG |
| | Chim3_pET_rv | CGGGATCCTTAGTAAATCTTTTCTTCTTCCTTC |
| | 1r-Chim3-opt-o(Chim3- | TGCCCTGAGGAAAGCGCGCACGTAATTC |
| | opt) | |
| | 2f-Chim3-opt-o(Chim3- | TGCGCGCTTTCCTCAGGGCAACTTAATC |
| | opt) | |
| | 1f-Assembly-o(Vec) | TGACGATAAGGATCGATGGGGATCCATGACCAAATACAAA |
| | 1r-Assembly-o(Vec) | TATGGTACCAGCTGCAGATCTCGAGTTAGTAAATCTTTTCTTC |
| Chimera 4 | GTDopt_pEt_fw | GGGAATTCCATATGATGACCAAATACAAAAATG |
| | Chim3_pET_rv | CGGGATCCTTAGTAAATCTTTTCTTCTTCCTTC |
| | 1r-Chim4_GTD- | CGATTTTGCGCCCATATTGTAACAACTTTTGA |
| | o(Chim4_GTC) | |
| | 2f-Chim4_GTC- | ACAATATGGGCGCAAAATCGTCGTAGTC |
| | o(Chim4_GTD) | |
| | 1f-Assembly-o(Vec) | TGACGATAAGGATCGATGGGGATCCATGACCAAATACAAA |
| | 1r-Assembly-o(Vec) | TATGGTACCAGCTGCAGATCTCGAGTTAGTAAATCTTTTCTTC |

To establish expression hosts purified pDrive::GT vectors were incubated with respective endonucleases (Table A1) and the fragments of interest were purified from Agarose after gel electrophoresis. Alternatively, the amplified and purified PCR product was directly incubated with respective endonucleases and purified from agarose gel after electrophoresis. The fragments were ligated into prepared pET19b or pTrcHisA plasmids and competent E. coli Rosetta gami 2 (DE3) were transformed by heat shock. Positive clones were verified after overnight growth by direct colony PCR using T7 promoter primers and the GT gene reverse primers, respectively.

Altogether, seven production strains were established:

| | | |
|---|---|---|
| 1. | PetC | E. coli Rosetta gami 2 (DE3) pET19b::GTC |
| 2. | PetD | E. coli Rosetta gami 2 (DE3) pET19b::GTD |
| 3. | PetF | E. coli Rosetta gami 2 (DE3) pET19b::GTF |
| 4. | PetS | E. coli Rosetta gami 2 (DE3) pET19b::GTS |
| 5. | PetChim1fs | E. coli Rosetta gami 2 (DE3) pET19b::Chimera 1 |
| | | frameshift |
| 6. | PetChim3 | E. coli Rosetta gami 2 (DE3) pET19b::Chimera 3 |
| 7. | PetChim4 | E. coli Rosetta gami 2 (DE3) pET19b::Chimera 4 |

### Example A3—Production of Rhamnosylated Flavonoids in Biotransformations

Three kinds of whole cell bioconversion (biotransformation) were performed. All cultures were inoculated 1/100 with overnight pre-cultures of the respective strain. Pre-cultures were grown at 37° C. in adequate media and volumes from 5 to 100 mL supplemented with appropriate antibiotics.

### 1. Analytical Small Scale and Quantitative Shake Flask Cultures

For analytical activity evaluations, 20 mL biotransformations were performed in 100 mL Erlenmeyer flasks while quantitative biotransformations were performed in 500 mL cultures in 3 L Erlenmeyer flasks. Bacterial growth was accomplished in complex media, e.g. LB, TB, and RM, or in M9 supplemented with appropriate antibiotics at 28° C. until an OD₆₀₀ of 0.8. Supplementation of 50 or 100 μM Isopropyl-β-D-thiogalactopyranoside (IPTG) induced gene expression overnight (16 h) at 17° C. and 175 rpm shaking. Subsequently, a polyphenolic substrate, e.g. Naringenin, Hesperetin or else, in concentrations of 200-800 μM was added to the culture. Alternatively, the polyphenolic substrate was supplemented directly with the IPTG. A third alternative was to harvest the expression cultures by mild centrifugation (5.000 g, 18° C., 10 min) and suspend in the same volume of PBS, supplied with 1% (w/v) glucose, optionally biotin and/or thiamin, each at 1 mg/L, the appropriate antibiotic and the substrate in above mentioned concentrations. All biotransformation reactions in 3 L shake flasks were incubated at 28° C. up to 48 h at 175 rpm.

2. Quantitative bioreactor (fermenter) cultures

In order of a monitorable process bioconversions were performed in volumes of 0.5 L in a Dasgip fermenter system (Eppendorf, Germany) The whole process was run at 26 to 28° C. and kept at pH 7.0. The dissolved oxygen (DO) was kept at 30% minimum. During growth the DO rises due to carbohydrate consumption. At DO of 50% an additional feed with glucose was started with 1 mL/h following the equation

y=e^{0.1x}

whereby y represents the added volume (mL) and x the time (h).

For cell growth the bacterial strains were grown in LB, TB, RM or M9 overnight. At OD₆₀₀ of 10 to 50 50 μM of IPTG and the polyphenolic substrate (400-1500 μM) were added to the culture. The reaction was run for 24 to 48 h.

All bioconversion reactions were either stopped by cell harvest through centrifugation (13,000 g, 4° C., 20 min) followed by sterile filtration with a 0.22 μM PES membrane (Steritop™, Carl Roth, Germany) Alternatively, cultures were harvested by hollow fibre membrane filtration techniques, e.g. TFF Centramed system (Pall, USA). Supernatants were purified directly or stored short-term at 4° C. (without light).

### Qualitative Analyses of Biotransformation Reactions and Products

Biotransformation products were determined by thin layer chromatography (TLC) or by HPLC.

For qualitative TLC analysis, 1 mL culture supernatant was extracted with an equal amount ethyl acetate (EtOAc). After centrifugation (5 min, 3,000 g) the organic phase was transferred into HPLC flat bottom vials and was used for TLC analysis. Samples of 20 μL were applied on 20×10 cm² (HP)TLC silica 60 F₂₅₄ plates (Merck KGaA, Darmstadt, Germany) versus 200 pmol of reference flavonoids by the ATS 4 (CAMAG, Switzerland). To avoid carryover of substances, i.e. prevent false positives, samples were spotted with double syringe rinsing in between. The sampled TLC plates were developed in EtOAc/acetic acid/formic acid/water (EtOAc/HAc/HFo/H₂O) 100:11:11:27. After separation the TLC plates were dried in hot air for 1 minute. The chromatograms were read and absorbances of the separated bands were determined densitometrically depending on the absorbance maximum of the educts at 285 to 370 nm (D2) by a TLC Scanner 3 (CAMAG, Switzerland).

### Analytical HPLC Conditions

HPLC analytics were performed on a VWR Hitachi LaChrom Elite device equipped with diode array detection.

### Column: Agilent Zorbax SB-C18 250×4.6 mm, 5 μM

Flowrate: 1 mL/min
Mobile phases: A: H₂O+0.1% Trifluoro acetic acid (TFA), B: ACN+0.1% TFA 0-5′:5% B, 5-15′: 15% B, 15-25′: 25% B, 25-25′: 35% B, 35-45′: 40%, 45-55′ 100% B, 55-63′: 5% B
Sample injection volume 100-500 μL
MS and MS/MS analyses were obtained on a microOTOF-Q with electrospray ionization (ESI) from Bruker (Bremen, Germany) The ESI source was operated at 4000 V in negative ion mode. Samples were injected by a syringe pump and a flow rate of 200 μL/min.

In order to purify the polyphenolic glycosides two different purification procedures were applied successfully.

1. Extraction and subsequent preparative HPLC
1.1 In liquid-liquid extractions bioconversion culture supernatants were extracted twice with half a volume of iso-butanol or EtOAc.
1.2 In solid phase extractions (SPE) supernatants were first bound on suitable polymeric matrices, e.g. Amberlite XAD resins or silica based functionalized phases, e.g. C-18, and subsequently eluted with organic solvents, e.g. ACN, methanol (MeOH), EtOAc, dimethyl sulfoxide (DMSO) et al. or with suitable aqueous solutions thereof, respectively.
Organic solvents were evaporated and the residuum completely dissolved in water-acetonitrile (H₂O-ACN) 80:20. This concentrate was further processed by HPLC as described below.
   2. Direct fractionation by preparative HPLC
Sterile filtered (0.2 μm) biotransformation culture supernatants or pre-concentrated extracts were loaded on adequate RP18 columns (5 μm, 250 mm) and fractionated in a H₂O-ACN gradient under following general conditions:
System: Agilent 1260 Infinity HPLC system.
Column: ZORBAX SB-C18 prepHT 250×21.2 mm, 7 μm.
Flowrate: 20 mL/min
Mobile Phase: A: Water+0.1 formic acid
   B: ACN+0.1 formic acid

| | | |
|---|---|---|
| Gradient: | 0-5 min | 5-30% B |
| | 5-10 min | 30% B |
| | 10-15 min | 35% B |
| | 15-20 min | 40% B |
| | 20-25 min | 100% B |

Fractions containing the polyphenolic glycosides were evaporated and/or freeze dried. Second polishing steps were performed with a pentafluor-phenyl (PFP) phase by HPLC to separate double peaks or impurities.

The rhamnose transferring activity was shown with enzymes GTC, GTD, GTF and GTS and the three chimeric enzymes chimera 1 frameshift, chimera 3 and chimera 4 in preparative and analytical biotransformation reactions. The enzymes were functional when expressed in different vector systems. GT-activity could be already determined in cloning systems, e.g. E. coli DH5α transformed with pDrive vector (Qiagen, Germany) carrying GT-genes. E. coli carrying pBluescript II SK+ with inserted GT-genes also was actively glycosylating flavonoids. For preparative scales the production strains PetC, PetD, PetF, PetS, PetChim1fs, PetChim3 and PetChim4 were successfully employed. Products were determined by HPLC, TLC, LC-MS and NMR analyses.

Biotransformation of the Flavanone Hesperetin Using E. coli Rosetta Gami 2 (DE3) pET19b::GTC (PetC)

In a preparative scale reaction hesperetin (3′,5,7-Trihydroxy-4′-methoxyflavanone, 2,3-dihydro-5,7-dihydroxy-2-(3-hydroxy-4-methoxyphenyl)-4H-1-benzopyran-4-one, CAS No. 520-33-2) was converted. The biotransformation was performed following general preparative shake flask growth and bioconversion conditions.

The bioconversion of hesperetin (>98%, Cayman, USA) was monitored by HPLC analyses of 500 μL samples taken at start (T=0), 3 h and 24 h reaction at 28° C. The culture supernatant was loaded directly via pump flow to a preparative RP18 column (Agilent, USA). Stepwise elution was performed and seven fractions were collected according to FIG. 10 and table A2.

All seven fractions subsequently were analyzed by HPLC and ESI-Q-TOF MS analyses. MS analyses in negative ion mode revealed fraction 3 and fraction 6 to contain a compound each with the molecular weight of 448 Da corresponding to hesperetin-O-rhamnoside (C₂₂H₂₄O₁₀) (FIGS. 11 and 12 table A2). To further purify the two compounds fractions 3 and 6 were lyophilized and dissolved in 30% ACN.

Final purification was performed by HPLC using a PFP column The second purification occurred on a Hypersil Gold PFP, 250×10 mm, 5 μm purchased from Thermo Fischer Scientific (Langerwehe, Germany) and operated at a flow rate of 6 mL/min (Mobile Phase: A: Water, B: ACN, linear gradient elution (0′-8′:95%-40% A, 8′-13′:100% B)(FIG. 13). Subsequently, ESI-TOF MS analyses of the PFP fractions identified the target compounds designated HESR1 and HESR2 in respective fractions (table A3).

After lyophilization NMR analyses elucidated the molecular structure of HESR1 and HESR2, respectively (Example B-2). HESR1 turned out to be the hesperetin-5-O-α-L-rhamnoside and had a RT of 28.91 min in analytical HPLC conditions. To this point, this compound has ever been isolated nor synthesized before.

TABLE A2
Fractionation of hesperetin bioconversion by prepLC separation

| Frac | | | Volume | BeginTime | EndTime | | |
|---|---|---|---|---|---|---|---|
| # # | Well | Location | [μl] | [min] | [min] | Description | ESI-MS |
| 1 1 | Vial | 201 | 20004.17 | 3.4999 | 4.5001 Time | | |
| 2 1 | Vial | 202 | 58004.17 | 4.9999 | 7.9001 Time | | |
| 3 1 | Vial | 203 | 17804.17 | 7.9999 | 8.8901 Time | HESR1 | 448 |
| 4 1 | Vial | 204 | 20791.67 | 8.9505 | 9.9901 Time | | |
| 5 1 | Vial | 205 | 39012.50 | 10.0495 | 12.0001 Time | | |
| 6 1 | Vial | 206 | 38004.17 | 12.0999 | 14.0001 Time | HESR2 | 448 |
| 7 1 | Vial | 207 | 40004.17 | 17.9999 | 20.0001 Time | | |

TABLE A3
Peak table of PFP-HPLC of fraction 3 hesperetin bioconversion

| | | Width | | | | |
|---|---|---|---|---|---|---|
| RT [min] | Type | [min] | Area | Height | Area % | Name |
| 2.030 | BB | 0.1794 | 866.4182 | 75.7586 | 3.9105 | |
| 2.507 | BV | 0.1642 | 493.0764 | 43.5284 | 2.2254 | |
| 2.686 | VV | 0.0289 | 20.4545 | 9.5811 | 0.0923 | |
| 2.772 | VB | 0.0861 | 85.4639 | 15.0938 | 0.3857 | |
| 2.939 | BB | 0.0806 | 119.9032 | 23.8914 | 0.5412 | |
| 3.264 | BV | 0.1016 | 16549.5371 | 2365.6169 | 74.6942 | HESR1 |
| 3.488 | VV | 0.0977 | 957.1826 | 140.0522 | 4.3201 | |
| 3.742 | VB | 0.0932 | 2007.7089 | 320.0400 | 9.0615 | |
| 4.047 | BB | 0.0816 | 74.1437 | 14.5014 | 0.3346 | |
| 4.467 | BB | 0.1241 | 190.8758 | 23.6774 | 0.8615 | |
| 5.238 | BV | 0.1326 | 121.1730 | 13.5104 | 0.5469 | |
| 5.501 | VB | 0.1617 | 315.1474 | 27.9130 | 1.4224 | |
| 6.192 | BV | 0.1654 | 43.3605 | 3.8503 | 0.1957 | |
| 10.368 | VV | 0.4019 | 296.8163 | 9.8411 | 1.3396 | |
| 12.464 | VB | 0.1204 | 15.1287 | 1.7240 | 0.0683 | |

### Biotransformation of the Flavanone Naringenin Using PetC in a Preparative Shake Flask Culture

Naringenin (4′,5,7-Trihydroxyflavanone, 2,3-dihydro-5,7-dihydroxy-2-(4-hydroxyphenyl)-4H-1-benzopyran-4-one, CAS No. 67604-48-2) was converted in a preparative scale reaction. The biotransformation was performed following general preparative shake flask growth and bioconversion conditions.

The bioconversion of naringenin (98%, Sigma-Aldrich, Switzerland) was controlled by HPLC analyses of a 500 μL sample after 24 h reaction. The culture supernatant was directly loaded via pump flow to a preparative RP18 column. Stepwise elution was performed and seven fractions were collected according to table A4.

All seven fractions subsequently were analyzed by HPLC and ESI-TOF MS analyses. MS analyses in negative ion mode revealed fraction 3 and fraction 5 to contain a compound each with the molecular weight of 418 Da which is the molecular weight of naringenin-O-rhamnoside (C₂₁H₂₂O₉)(table A4). The two compounds designated NR1 and NR2 were lyophilized. HPLC analysis in analytical conditions revealed RTs of approx. 27.2 min for NR1 and 35.7 min for NR2, respectively. NMR analyses elucidated the molecular structure of NR1 (Example B-3). NR1 was identified to be an enantiomeric 1:1 mixture of S- and R-naringenin-5-O-α-L-rhamnoside (N5R). Since the used precursor also was composed of both enantiomers the structure analysis proved that both isomers were converted by GTC. To our knowledge this is the first report that naringenin-5-O-α-L-rhamnoside has ever been biosynthesized. The compound was isolated from plant material (Shrivastava (1982) Ind J Chem Sect B 21(6):406-407). However, the rare natural occurrence of this scarce flavonoid glycoside has impeded any attempt of an industrial application.

In contrast, the first time bioconversion of naringenin-5-O-α-L-rhamnoside opens the way of a biotechnological production process for this compound. Until now the biotechnological production was only shown for e.g. naringenin-7-O-α-L-xyloside and naringenin-4′-O-β-D-glucoside (Simkhada (2009) Mol. Cells 28:397-401, Werner (2010) Bioprocess Biosyst Eng 33:863-871).

TABLE A4
Fractionation of naringenin bioconversion by prepLC separation

| Frac | | | Volume | BeginTime | EndTime | | |
|---|---|---|---|---|---|---|---|
| # # | Well | Location | [μl] | [min] | [min] | Description | ESI-MS |
| 1 1 | Vial | 201 | 31518.75 | 4.6963 | 6.4407 Time | | |
| 2 1 | Vial | 202 | 17328.75 | 6.5074 | 7.4634 Time | | |
| 3 1 | Vial | 203 | 34638.75 | 7.5301 | 9.4478 Time | NR1 | 418 |
| 4 1 | Vial | 204 | 43905.00 | 9.5130 | 11.9455 Time | | |
| 5 1 | Vial | 205 | 115995.00 | 12.0109 | 18.4484 Time | NR2 | 418 |
| 6 1 | Vial | 206 | 71111.25 | 18.5151 | 22.4590 Time | | |
| 7 1 | Vial | 207 | 80047.50 | 22.5242 | 26.9647 Time | | |

Biotransformation of Naringenin Using E. coli Rosetta Gami 2 (DE3)pET19b::GTC (PetC) in a Monitored Bioreactor System

Next to production of naringenin rhamnosides in shake flask cultures a bioreactor process was successfully established to demonstrate applicability of scale-up under monitored culture parameters.

In a Dasgip fermenter system (Eppendorf, Germany) naringenin was converted in four fermenter units in parallel under conditions stated above.

At an OD₆₀₀ of 50 expression in PetC was induced by IPTG while simultaneously supplementation of 0.4 g of naringenin (98% CAS No. 67604-48-2, Sigma-Aldrich, Switzerland) per unit was performed. Thus, the final concentration was 2.94 mM of substrate.

After bioconversion for 24 h the biotransformation was finished and centrifuged. Subsequently, the cell free supernatant was extracted once with an equal volume of iso-butanol by shaking intensively for one minute. Preliminary extraction experiments with defined concentrations of naringenin rhamnosides revealed an average efficiency of 78.67% (table A5).

HPLC analyses of the bioreactor reactions indicated that both products, NR1 (RT 27,28′) and NR2 (RT 35.7′), were built successfully (FIG. 16). ESI-MS analyses verified the molecular mass of 418 Da for both products. Quantitative analysis of the bioconversion products elucidated the reaction yields. Concentration calculations were done from peak areas after determination regression curves of NR1 and NR2 (table A6). NR1 yielded an average product concentration of 393 mg/L, NR2 as the byproduct yielded an average 105 mg/L.

TABLE A5
Extraction of naringenin biotransformation
products from supernatant with iso-butanol

| | |
|---|---|
| Extraction mit iso-butanol | 1 ml/1 mL 1′ shaking |

| % | Mean | Loss % | Std Dev. |
|---|---|---|---|
| 75.75160033 | 78.6707143 | 21.32928571 | 2.73747541 |
| 82.49563254 | | | |
| 76.42705533 | | | |
| 80.00856895 | | | |

TABLE A6
HPLC chromatogram peak area and resulting
product concentrations of NR1 and NR2

| | NR1 | | NR2 | |
|---|---|---|---|---|
| | | Concentration | | Concentration |
| | Peak area | mg/mL | Peak area | mg/mL |
| Unit 1 26° C. 24 h | 232620332 | 0.33231476 | 64179398 | 0.091684854 |
| Unit 2 28° C. 24 h | 192866408 | 0.27552344 | 57060698 | 0.081515283 |
| Unit 3 26° C. 24 h | 235176813 | 0.335966876 | 61065093 | 0.087235847 |
| Unit 4 28° C. 24 h | 204937318 | 0.292767597 | 49803529 | 0.071147899 |
| Unit 1 26° C. 24 h | 232620332 | 0.422412283 | 64179398 | 0.116542547 |
| Unit 2 28° C. 24 h | 192866408 | 0.350223641 | 57060698 | 0.103615791 |
| Unit 3 26° C. 24 h | 235176813 | 0.427054564 | 61065093 | 0.110887321 |
| Unit 4 28° C. 24 h | 204937318 | 0.372143052 | 49803529 | 0.090437591 |
| Average | | 0.392958385 | | 0.105370812 |

Biotransformation of Narengenin Using E. coli Rosetta Gami 2 (DE3)pET19b::GTC (PetC), E. coli Rosetta Gami 2 (DE3) pET19b::GTD (PetD), E. coli Rosetta Gami 2 (DE3) pET19b::GTF (PetF), E. coli Rosetta Gami 2 (DE3) pET19b::GTS (PetS), E. coli Rosetta Gami 2 (DE3) pET19b::Chimera 1 Frameshift (PetChim1fs), E. coli Rosetta Gami 2 (DE3)pET19b::Chimera 3 (PetChim3) and E. coli Rosetta Gami 2 (DE3)pET19b::Chimera 4 (PetChim4), Respectively

To determine the regio specificities of GTC, GTD, GTF and GTS as well as the three chimeric enzymes chimera 1 frameshift, chimera 3 and chimera 4 biotransformations were performed in 20 mL cultures analogously to preparative flask culture bioconversions using naringenin as a substrate among others. To purify the formed flavonoid rhamnosides, the supernatant of the biotransformation was loaded on a C₆H₅ solid phase extraction (SPE) column. The matrix was washed once with 20% acetonitrile. The flavonoid rhamnosides were eluted with 100% acetonitrile. Analyses of the biotransformations were performed using analytical HPLC and LC-MS. For naringenin biotransformations analyses results of the formed products NR1 and NR2 of each production strain are listed in Table A7 and A8, respectively.

TABLE A7
Formed NR1 products in bioconversions of naringenin
with different production strains

| strain | NR1 retention time [min] HPLC | ESI-MS | ESI-MSMS |
|---|---|---|---|
| PetC | 27.32 | 418 | 272 |
| PetD | 27.027 | 418 | 272 |
| PetF | 26.627 | 418 | 272 |
| PetS | 26.833 | 418 | 272 |
| PetChim1fs | 26.673 | 418 | 272 |
| PetChim3 | 26.72 | 418 | 272 |
| PetChim4 | 26.727 | 418 | 272 |

TABLE A8
Formed NR2 products in bioconversions of naringenin
with different production strains

| strain | NR2 retention time [min] HPLC | ESI-MS | ESI-MSMS |
|---|---|---|---|
| PetC | 35.48 | 418 | 272 |
| PetD | 35.547 | 418 | 272 |
| PetF | 35.26 | 418 | 272 |
| PetS | 35.28 | 418 | 272 |
| PetChim1fs | 35.080 | 418 | 272 |
| PetChim3 | 35.267 | 418 | 272 |
| PetChim4 | 35.267 | 418 | 272 |

### Biotransformation of the Flavanone Homoeriodictyol (HED) Using PetC

In preparative scale HED (5,7-Dihydroxy-2-(4-hydroxy-3-methoxyphenyl)-4-chromanone, CAS No. 446-71-9) was glycosylated by PetC. The biotransformation was performed following general preparative shake flask growth and bioconversion conditions.

The bioconversion of HED was monitored by HPLC analyses. The culture supernatant was loaded directly via pump flow to a preparative RP18 column (Agilent, USA). Stepwise elution was performed and nine fractions were collected according to table A5.

All nine fractions subsequently were analyzed by HPLC and ESI-TOF MS analyses. MS analyses of fractions 5 and 8 in negative ion mode showed that both contained a compound with the molecular weight of 448 Da which corresponded to the size of a HED-O-rhamnoside and were designated HEDR1 and HEDR3. MS analysis of fraction 7 (HEDR2) gave a molecular weight of 434 Da. However, ESI MS/MS analyses of all three fractions identified a leaving group of 146 Da suggesting a rhamnosidic residue also in fraction 7.

After HPLC polishing by a (PFP) phase and subsequent lyophilization the molecular structure of HEDR1 was solved by NMR analysis (Example B-1). HEDR1 (RT 28.26 min in analytical HPLC) was identified as the pure compound HED-5-O-α-L-rhamnoside.

TABLE A9
Fractionation of HED bioconversion by prepLC separation

| Frac | | | Volume | BeginTime | EndTime | Description | |
|---|---|---|---|---|---|---|---|
| # # | Well | Location | [μl] | [min] | [min] | [compound] | ESI-MS |
| 1 1 | Vial | 201 | 22503.75 | 5.0999 | 6.3501 Time | | |
| 2 1 | Vial | 202 | 28593.75 | 6.4115 | 8.0001 Time | | |
| 3 1 | Vial | 203 | 34927.50 | 8.0597 | 10.0001 Time | | |
| 4 1 | Vial | 204 | 20141.25 | 10.0611 | 11.1801 Time | | |
| 5 1 | Vial | 205 | 13695.00 | 11.2392 | 12.0001 Time | HEDR1 | 448 |
| 6 1 | Vial | 206 | 34931.25 | 12.0594 | 14.0001 Time | | |
| 7 1 | Vial | 207 | 25203.75 | 15.5999 | 17.0001 Time | HEDR2 | 434 |
| 8 1 | Vial | 208 | 38246.25 | 17.0753 | 19.2001 Time | HEDR3 | 448 |
| 9 1 | Vial | 209 | 66603.75 | 19.2999 | 23.0001 Time | HED | 302 |

### Biotransformation Reactions Using PetC of the Isoflavone Genistein Using PetC

In preparative scale genistein (4′,5,7-Trihydroxyisoflavone, 5,7-dihydroxy-3-(4-hydroxyphenyl)chromen-4-one, CAS No. 446-72-0) was glycosylated in bioconversion reactions using PetC. The biotransformation was performed in PBS following general preparative shake flask growth and bioconversion conditions.

The bioconversion of genistein was monitored by HPLC analyses. The genistein aglycon showed a RT of approx. 41 min. With reaction progress four peaks of reaction products (GR1-4) with RTs of approx. 26 min, 30 min, 34.7 min, and 35.6 min accumulated in the bioconversion (table A10). The reaction was stopped by cell harvest after 40 h and in preparative RP18 HPLC stepwise elution was performed. All fractions were analyzed by HPLC and ESI-Q-TOF MS analyses.

Fractions 3, 4, and 5, respectively, showed the molecular masses of genistein rhamnosides in MS analyses. Fraction 3 consisted of two separated major peaks (RT 26 min and 30 min) Fraction 4 showed a double peak of 34.7 min and 35.6 min, fraction 5 only the latter product peak at RT 35.6 min. Separate MS analyses of the peaks in negative ion mode revealed that all peaks contained compounds with the identical molecular masses of 416 which corresponded to the size of genistein-O-rhamnosides. NMR analysis of GR1 identified genistein-5,7-di-O-α-L-rhamnoside (Example B-9).

### Biotransformation of the Isoflavone Biochanin A Using PetC

In preparative scale biochanin A (5,7-dihydroxy-3-(4-methoxyphenyl)chromen-4-one, CAS No. 491-80-5) was glycosylated in bioconversion reactions using PetC. The biotransformation was performed following general preparative shake flask growth and bioconversion conditions. The bioconversion of biochanin A was monitored by HPLC. The biochanin A aglycon showed a RT of approx. 53.7 min With reaction progress three product peaks at approx. 32.5′, 36.6′, and 45.6′ accumulated in the bioconversion (table A10). These were termed BR1, BR2, and BR3, respectively. The reaction was stopped by cell harvest after 24 h through centrifugation (13,000 g, 4° C.). The filtered supernatant was loaded to a preparative RP18 column and fractionated by stepwise elution. All fractions were analyzed by HPLC and ESI-Q-TOF MS analyses.

The PetC product BR1 with a RT of 32.5 min was identified by NMR as the 5,7-di-O-α-L-rhamnoside of biochanin A (Example B-4). NMR analysis of BR2 (RT 36.6′) gave the 5-O-α-L-rhamnoside (example B-5). In accordance to 5-O-α-L-rhamnosides of other flavonoids, e.g. HED-5-O-α-L-rhamnoside, BR2 was the most hydrophilic mono-rhamnoside with a slight retardation compared to HEDR1. Taking into account the higher hydrophobicity of the precursor biochanin A (RT 53.5′) due to less hydroxy groups and its C4′-methoxy function in comparison to a C4′-OH of genistein (RT 41′) the retard of BR2 compared to GR2 could be explained.

### Biotransformation of the Flavone Chrysin Using PetC

In preparative scale chrysin (5,7-Dihydroxyflavone, 5,7-Dihydroxy-2-phenyl-4-chromen-4-one, CAS No. 480-40-0) was glycosylated in bioconversion reactions using PetC. The biotransformation was performed following stated preparative shake flask conditions in PBS.

The bioconversion of chrysin was monitored by HPLC analyses. The chrysin aglycon showed a RT of 53.5 min. In PetC bioconversions three reaction product peaks accumulated in the reaction, CR1 at RT 30.6 min, CR2 at RT36.4 min, and CR3 at RT43.4, respectively (table A10). All products were analyzed by HPLC and ESI-Q-TOF MS analyses.

CR1 was further identified by NMR as the 5,7-di-O-α-L-rhamnoside of chrysin (Example B-6) and in NMR analysis CR2 turned out to be the 5-O-α-L-rhamnoside (Example B-7). Like BR2, CR2 was also less hydrophilic than the 5-O-rhamnosides of flavonoids with free OH-groups at ring C, e.g. hesperetin and naringenin, although CR2 was the most hydrophilic mono-rhamnoside of chrysin.

### Biotransformation of the Flavone Diosmetin Using PetC

Diosmetin (5,7-Trihydroxy-4′-methoxyflavone, 5,7-dihydroxy-2-(3-hydroxy-4-methoxyphenyl) chromen-4-one, CAS No. 520-34-3) was glycosylated in bioconversion reactions using PetC. The biotransformation was performed as stated before.

The bioconversion of diosmetin was monitored by HPLC. The diosmetin aglycon showed a RT of 41.5 min using the given method. With reaction progress three peaks of putative reaction products at 26.5′ (DR1), 29.1′ (DR2), and 36′ (DR3) accumulated (table A10).

The product DR2 with a RT of 29.1 min was further identified as the 5-O-α-L-rhamnoside of diosmetin (D5R) (Example B-10). DR1 was shown by ESI-MS analysis to be a di-rhamnoside of diosmetin. In accordance with the 5-O-α-L-rhamnosides of other flavonoids, e.g. hesperetin, DR2 had a similar retention in analytical RP18 HPLC-conditions.

Table A10 summarizes all reaction products of PetC biotransformations with the variety of flavonoid precursors tested.

TABLE A10
Compilation of applied precursors and corresponding rhamnosylated products

| | | | | NMR | Elucidated |
|---|---|---|---|---|---|
| Precursor | Products | RT [min] | ESI-MS | (Part B) | Structure |
| Homoeriodictyol | | 42.4 | 302.27 | | |
| | HEDR1 | 28.1 | 448.11 | B-1 | 5-O-α-L-rhamnoside |
| | HEDR2 | 34.6 | 434.13 | | |
| | HEDR3 | Double | 448.11 | | |
| | | Peak | | | |
| | | 35.8/36.4 | | | |
| Hesperetin | | 41.1 | 302.27 | | |
| | HESdiR | 26.3 | 594.12 | — | 3′,5-di-O-α-L-rhamnoside |
| | HESR1 | 28.2 | 448.15 | B-2 | 5-O-α-L-rhamnoside |
| | HESR | 2 | 448.15 | | |
| Naringenin | | 40.8 | 272.26 | | |
| | NR1 | 27.2 | 418.1 | B-3 | 5-O-α-L-rhamnoside |
| | NR2 | 25.7 | 418.1 | | |
| Biochanin A | | 53.7 | 284.26 | | |
| | BR1 | 32.5 | — | B-4 | 5,7-di-O-α-L-rhamnoside |
| | BR2 | 36.6 | 430.15 | B-5 | 5-O-α-L-rhamnoside |
| | BR3 | 45.6 | 430.15 | — | |
| Chrysin | | 53.0 | 254.24 | | |
| | CR1 | 30.6 | — | B-6 | 5,7-di-O-α-L-rhamnoside |
| | CR2 | 36.4 | 400.14 | B-7 | 5-O-α-L-rhamnoside |
| | CR3 | 43.4 | 400.14 | — | |
| Silibinin | | 39.8 | 482.44 | | |
| | SR1 | 32.5 | 628.15 | B-8 | 5-O-α-L-rhamnoside |
| Genistein | | 40.8 | 270.24 | | |
| | GR1 | 25.9 | — | B-9 | 5,7-di-O-α-L-rhamnoside |
| | GR2 | 30.0 | 416.15 | | |
| | GR3 | 34.7 | 416.15 | | |
| | GR4 | 35.6 | 416.15 | | |
| Diosmetin | | 41.5 | 300.26 | | |
| | DR1 | 26.5 | — | — | Di-O-α-L-rhamnoside |
| | DR2 | 29.1 | 446.15 | B-10 | 5-O-α-L-rhamnoside |
| | DR3 | 36.0 | 446.15 | | |

### Part B: NMRanalyses of the Rhamnosylated Flavonoids

The following Examples were prepared according to the procedure described above in Part A.

### Example B-1: HED-5-O-α-L-rhamnoside

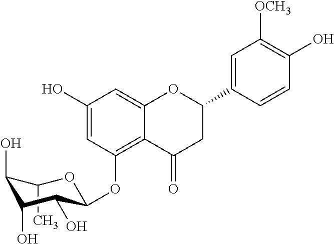

¹H NMR ((600 MHz Methanol-d₄): δ=7.06 (d, J=2.0 Hz, 1H), 7.05 (d, J=2.1 Hz, 1H), 6.91 (dt, J=8.2, 2.1, 0.4 Hz, 1H), 6.90 (ddd, J=8.1, 2.0, 0.6 Hz, 1H), 6.81 (d, J=8.1 Hz, 1H), 6.80 (d, J 8.1 Hz, 1H), 6.32 (d, J=2.3 Hz, 1H), 6.29 (d, J=2.3 Hz, 1H), 6.09 (t, J=2.3 Hz, 2H), 5.44 (d, J=1.9 Hz, 1H), 5.40 (d, J=1.9 Hz, 1H), 5.33 (dd, J=7.7, 2.9 Hz, 1H), 5.31 (dd, J=8.1, 3.0 Hz, 1H), 4.12 (ddd, J 11.2, 3.5, 1.9 Hz, 2H), 4.08 (dd, J=9.5, 3.5 Hz, 1H), 4.05 (dd, J=9.5, 3.5 Hz, 1H), 3.87 (s, 3H), 3.87 (s, 3H), 3.69-3.60 (m, 2H), 3.46 (td, J=9.5, 5.8 Hz, 2H), 3.06-3.02 (m, 1H), 3.02-2.98 (m, 1H), 2.64 (ddd, J=16.6, 15.5, 3.0 Hz, 2H), 1.25 (d, J=6.2 Hz, 3H), 1.23 (d, J=6.3 Hz, 3H).

### Example B-2: Hesperetin-5-O-α-L-rhamnoside

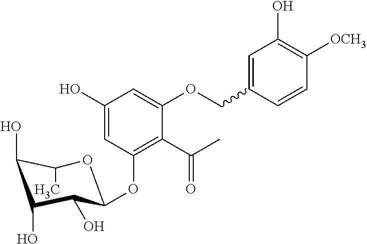

¹H-NMR (400 MHz, DMSO-d₆): δ=1.10 (3H, d, J=6.26 Hz, CH₃), 2.45 (m, H-3(a), superimposed by DMSO), 2.97 (1H, dd, J=12.5, 16.5 Hz, H3(b)), 3.27 (1H, t, 9.49 Hz, H(b)), 3.48 (m, H(a), superimposed by HDO), 3.76 (3H, s, OCH3), 3.9-3.8 (2H, m, H(c), Hd), 5.31 (1H, d, 1.76 Hz, He), 5.33 (1H, dd, 12.5, 2.83 Hz, H2), 6.03 (1H, d, 2.19 Hz, H6/H8), 6.20 (1H, d, 2.19 Hz, H6/H8), 6.86 (1H, dd, 8.2, 2.0 Hz, H6′), 6.90 (1H, d, 2.0 Hz, H2′), 6.93 (1H, d, 8.2 Hz, H5′)

### Example B-3: Naringenin-5-O-α-L-rhamnoside

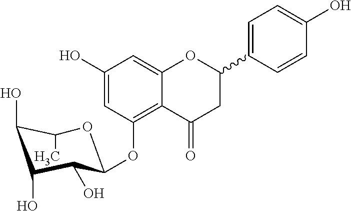

¹H NMR (600 MHz, DMSO-d₆): δ=7.30 (d, J=6.9 Hz, 2H), 7.29 (d, J=6.9 Hz, 2H), 6.79 (d, J=8.6 Hz, 2H), 6.78 (d, J=8.6 Hz, 2H), 6.22 (d, J=2.3 Hz, 1H), 6.20 (d, J=2.2 Hz, 1H), 6.02 (d, J=2.2 Hz, 1H), 6.01 (d, J=2.2 Hz, 1H), 5.38 (dd, J=12.7, 3.1 Hz, 1H), 5.35 (dd, J=13.0, 2.5 Hz, 1H), 5.31 (d, J=1.8 Hz, 1H), 5.27 (d, J=1.9 Hz, 1H), 3.90-3.88 (m, 1H), 3.88-3.85 (m, 1H), 3.85-3.80 (m, 2H), 3.50 (dq, J=9.2, 6.2 Hz, 1H), 3.48 (dq, J=9.1, 6.2 Hz, 1H), 3.29 (t, J=9.8 Hz, 2H), 3.07-2.98 (m, 2H), 2.55-2.48 (m, 2H), 1.12 (d, J=6.2 Hz, 3H), 1.10 (d, J=6.2 Hz, 3H).

¹³C NMR (151 MHz, DMSO-d₆): δ=187.75, 187.71, 164.04, 163.92, 163.80, 158.33, 158.23, 157.48, 157.44, 129.26, 129.24, 129.18, 129.15, 128.07, 128.00, 115.00, 105.19, 105.06, 98.58, 98.44, 97.25, 96.85, 96.77, 96.64, 78.03, 77.97, 71.67, 71.65, 69.98, 69.95, 69.66, 69.64, 44.78, 44.74, 17.80, 17.75.

### Example B-4: Biochanin A-5,7-di-O-α-L-rhamnoside

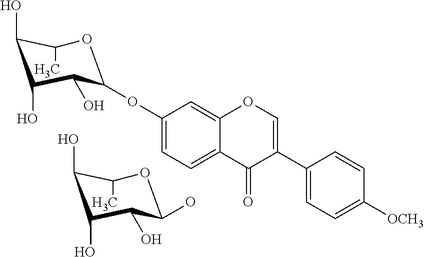

¹H NMR (400 MHz DMSO-d₆): δ=8.21 (s, 1H), 7.43 (d, J=8.5 Hz, 2H), 6.97 (d, J=8.6 Hz, 2H), 6.86 (d, J=1.8 Hz, 1H), 6.74 (d, J=1.8 Hz, 1H), 5.53 (d, J=1.6 Hz, 1H), 5.41 (d, J=1.6 Hz, 1H), 5.15 (s, 1H), 5.00 (s, 1H), 4.93 (s, 1H), 4.83 (s, 1H), 4.70 (s, 1H), 3.93 (br, 1H), 3.87 (br, 1H), 3.85 (br, 1H), 3.77 (s, 3H), 3.64 (dd, J=9.3, 3.0 Hz, 1H), 3.54 (dq, J=9.4, 6.4 Hz, 1H), 3.44 (dq, J=9.4, 6.4 Hz, 1H), 3.34 (br, 1H), 1.13 (d, J=6.1 Hz, 3H), 1.09 (d, J=6.1 Hz, 3H)

### Example B-5: Biochanin A 5-O-α-L-rhamnoside

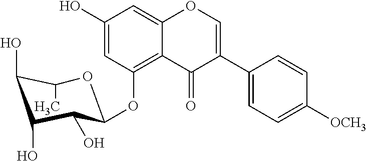

¹H NMR (400 MHz DMSO-d₆): δ=8.21 (s, 1H), 7.42 (d, J=8.7 Hz, 2H), 6.96 (d, J=8.7 Hz 2H), 6.55 (d, J=1.9 Hz, 1H), 6.48 (d, J=1.9 Hz, 1H), 5.33 (d, J=1.7 Hz, 1H), 5.1-4.1 (br, nH), 3.91 (br, 1H), 3.86 (d, J=9.7, 1H), 3.77 (s, 3H), 3.48 (br, superimposed by impurity, 1H), 3.44 (impurity), 3.3 (superimposed by HDO), 1.10 (d, J=6.2 Hz, 3H)

### Example B-6: Chrysin-di-5,7-O-α-L-rhamnoside

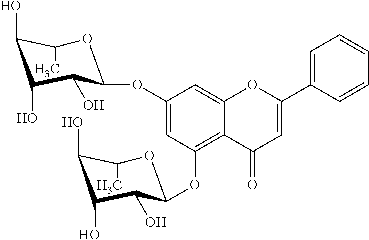

¹H NMR (400 MHz DMSO-d₆): δ=8.05 (m, 2H), 7.57 (m, 3H), 7.08 (s, 1H), 6.76 (d, J=2.3 Hz, 1H), 6.75 (s, 1H), 5.56 (d, J=1.6 Hz, 1H), 5.42 (d, J=1.6 Hz, 1H), 5.17 (s, 1H), 5.02 (s, 1H), 4.94 (s, 1H), 4.86 (s, 1H), 4.71 (s, 1H), 3.97 (br, 1H), 3.88 (dd, J=9.5, 3.1 Hz, 1H), 3.87 (br, 1H), 3.66 (dd, J=9.3, 3.4 Hz, 1H), 3.56 (dq, J=9.4, 6.2 Hz, 1H), 3.47 (dq, J=9.4, 6.2 Hz, 1H), 3.32 (superimposed by HDO, 2H), 1.14 (d, J=6.2 Hz, 3H), 1.11 (d, J=6.2 Hz, 3H)

### Example B-7: Chrysin-5-O-α-L-rhamnoside

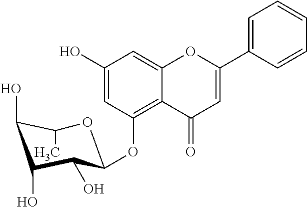

¹H NMR (400 MHz DMSO-d₆): δ=8.01 (m, 2H), 7.56 (m, 3H), 6.66 (s, 1H), 6.64 (d, J=2.1 Hz, 1H), 6.55 (d, J=2.1 Hz, 1H), 5.33 (d, J=1.5 Hz, 1H), 5.01 (s, 1H), 4.85 (d, J=4.7 Hz, 1H), 4.69 (s, 1H), 3.96 (br, 1H), 3.87 (md, J=8.2 Hz, 1H), 3.54 (dq, J=9.4, 6.2 Hz, 1H), 3.3 (superimposed by HDO), 1.11 (d, J=6.1 Hz, 3H)

### Example B-8: Silibinin-5-O-α-L-rhamnoside

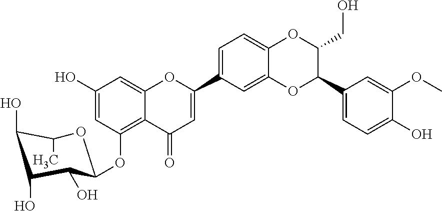

¹H NMR (400 MHz DMSO-d₆): δ=7.05 (dd, J=5.3, 1.9 Hz, 1H), 7.01 (br, 1H), 6.99 (ddd, J=8.5, 4.4, 1.8 Hz, 1H), 6.96 (dd, J=8.3, 2.3 Hz, 1H), 6.86 (dd, J=8.0, 1.8 Hz, 1H), 6.80 (d, J=8.0 Hz, 1H), 6.25 (d, J=1.9 Hz, 1H), 5.97 (dd, J=2.1, 3.7 Hz, 1H), 5.32 (d, J=1.6 Hz, 1H), 5.01 (d, J=11.2 Hz, 1H), 4.90 (d, J=7.3 Hz, 1H), 4.36 (ddd, J=11.2, 6.5, 4.6 Hz, 1H), 4.16 (ddd, J=7.6, 3.0, 4.6 Hz, 1H), 3.89 (m, 1H), 3.83 (br, 1H), 3.77 (d, J=1.8 Hz, 1H), 3.53 (m, 3H), 3.30 (superimposed by HDO, 3H), 1.13 (d, J=6.2 Hz, 3H)

### Example B-9: Genistein-5,7-di-O-α-L-rhamnoside

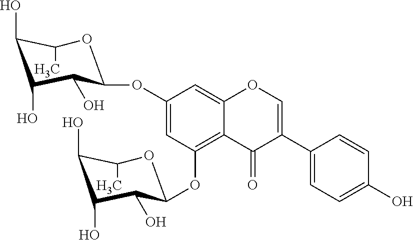

¹H NMR (400 MHz DMSO-d₆): δ=8.16 (s, 1H), 7.31 (d, J=8.4 Hz, 2H), 6.85 (d, J=2.1 Hz, 1H), 6.79 (d, J=8.4 Hz, 2H), 6.73 (d, J=2.1 Hz, 1H), 5.52 (d, J=1.8 Hz, 1H), 5.40 (d, J=1.8 Hz, 1H), 5.14 (d, J=3.8 Hz, 1H), 4.99 (d, J=3.8 Hz, 1H), 4.92 (d, J=5.2 Hz, 1H), 5.83 (d, J=5.2 Hz, 1H), 5.79 (d, J=5.5 Hz, 1H), 4.69 (d, J=5.5 Hz, 1H), 3.93 (br, 1H), 3.87 (br, 1H), 3.85 (br, 1H), 3.64 (br, 1H), 3.44 (m, 2H), 3.2 (superimposed by HDO, 2H), 1.12 (d, J=6.2 Hz, 3H), 1.09 (d, J=6.2 Hz, 3H)

### Example B-10: Diosmetin-5-O-α-L-rhamnoside

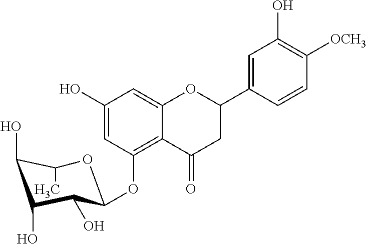

¹H NMR (600 MHz DMSO-d₆): δ=7.45 (dd, J=8.5, 2.3 Hz, 1H), 7.36 (d, J=2.3 Hz, 1H), 7.06 (d, J=8.6 Hz, 1H), 6.61 (d, J=2.3 Hz, 1H), 6.54 (d, J=2.3 Hz, 1H), 6.45 (s, 1H), 5.32 (d, J=1.7 Hz, 1H), 3.96 (dd, J=3.5, 2.0 Hz, 1H), 3.86 (m, 1H), 3.85 (s, 3H), 3.54 (dq, J=9.4, 6.3 Hz, 1H), 3.30 (superimposed by HDO, 1H), 1.11 (d, J=6.2, 3H)

### Part C: Solubility

FIG. 1 illustrates the amounts of Naringenin-5-rhamnoside recaptured from a RP18 HPLC-column after loading of a 0.2 μm filtered solution containing defined amounts up to 25 mM of the same. Amounts were calculated from a regression curve. The maximum water solubility of Naringenin-5-rhamnoside approximately is 10 mmol/L, which is equivalent to 4.2 g/L.

The hydrophilicity of molecules is also reflected in the retention times in a reverse phase (RP) chromatography. Hydrophobic molecules have later retention times, which can be used as qualitative determination of their water solubility.

HPLC-chromatography was performed using a VWR Hitachi LaChrom Elite device equipped with diode array detection under the following conditions:

Column: Agilent Zorbax SB-C18 250×4.6 mm, 5 μM, Flow 1 mL/min
Mobile phases: A: H₂O+0.1% Trifluoro acetic acid (TFA);

### B: ACN+0.1% TFA

Sample injection volume: 500 μL;

### Gradient: 0-5 min: 5% B, 5-15 min: 15% B, 15-25 min: 25% B, 25-25 min: 35% B, 35-45 min: 40%, 45-55 min: 100% B, 55-63 min: 5% B

TABLE B1
contains a summary of the retention times
according to FIGS. 2-9 and Example A-2.

| | N-5-O-α-L- | N-7-O-β-D- | N-4′-O-α-L- |
|---|---|---|---|
| Order of elution | rhamnoside | glucoside | rhamnoside |
| Retention time [min] | 27.3 | 30.9 | 36 |
| Order of elution | HED-5-O-α-L- | HED-4′-O-β-D- | HEDR3 |
| | rhamnoside | glucoside | |
| Retention time [min] | 28.3 | 30.1 | 35.8 |
| Order of elution | HES-5-O-α-L- | HESR2 | HES-7-O-β-D- |
| | rhamnoside | | glucoside |
| Retention time [min] | 28.9 | 36 | 31 |

Generally, it is well known that glucosides of lipophilic small molecules in comparison to their corresponding rhamnosides are better water soluble, e.g. isoquercitrin (quercetin-3-glucoside) vs. quercitrin (quercetin-3-rhamnosides). Table B1 comprehensively shows the 5-O-α-L-rhamnosides are more soluble than α-L-rhamnosides and β-D-glucosides at other positions of the flavonoid backbone. All the 5-O-α-L-rhamnosides eluted below 30 min in RP18 reverse phase HPLC. In contrast, flavanone glucosides entirely were retained at RTs above 30 min independent of the position at the backbone. In case of HED it was shown that among other positions, here C4′ and C7, the differences concerning the retention times of the α-L-rhamnosides were marginal, whereas the C5 position had a strong effect on it. This was an absolutely unexpected finding.

The apparent differences of the solubility are clearly induced by the attachment site of the sugar at the polyphenolic scaffold. In 4-on-5-hydroxy benzopyrans the OH-group and the keto-function can form a hydrogen bond. This binding is impaired by the substitution of an α-L-rhamnoside at C5 resulting in an optimized solvation shell surrounding the molecule. Further, in aqueous environments the hydrophilic rhamnose residue at the C5 position might shield a larger area of the hydrophobic polyphenol with the effect of a reduced contact to the surrounding water molecules. Another explanation would be that the occupation of the C5 position more effectively forms a molecule with a spatial separation a hydrophilic saccharide part and a hydrophobic polyphenolic part. This would result in emulsifying properties and the formation of micelles. An emulsion therefore enhances the solubility of the involved compound.

### Part D: Activity of Rhamnosylated Flavonoids

### Example D-1: Cytotoxicity of flavonoid-5-O-α-L-rhamnosides

To determine the cytotoxicity of flavonoid-5-O-α-L-rhamnosides tests were performed versus their aglycon derivatives in cell monolayer cultures. For this purpose concentrations ranging from 1 μM to 250 μM were chosen. The viability of normal human epidermal keratinocytes (NHEK) was twice evaluated by a MTT reduction assay and morphological observation with a microscope. NHEK were grown at 37° C. and 5% CO₂ aeration in Keratinocyte-SFM medium supplemented with epidermal growth factor (EGF) at 0.25 ng/mL, pituitary extract (PE) at 25 μg/mL and gentamycin (25 μg/mL) for 24 h and were used at the 3rd passage. For cytotoxicity testing, pre-incubated NHEK were given fresh culture medium containing a specific concentration of test compound and incubated for 24 h. After a medium change at same test concentration cells were incubated a further 24 h until viability was determined. Test results are given in Table B2 and illustrated in FIG. 10.

TABLE B2
Cytotoxicity of flavonoid-5-O-α-L-rhamnosides on normal human epidermal
keratinocytes

| | | | [μM] from stock solution at 100 mM in DMSO | | | | | | | |
|---|---|---|---|---|---|---|---|---|---|---|
| Compound | Control | | 1 | 2.5 | 5 | 10 | 25 | 50 | 100 | 250 |
| Hesperetin | | | | | | | | | | |
| Viability (%) | 98 | 98 | 103 | 98 | 107 | 101 | 106 | 106 | 98 | 54 |
| | 102 | 102 | 106 | 109 | 106 | 105 | 109 | 106 | 100 | 59 |
| Mean | 100 | | 105 | 103 | 106 | 103 | 108 | 106 | 99 | 57 |
| sd | 2 | | 2 | 8 | 1 | 3 | 2 | 0 | 1 | 4 |
| Morph. obs. | + | | + | + | + | + | + | + | +/− | +/− |
| Hes-5-Rha | | | | | | | | | | |
| Viability (%) | 95 | 85 | 86 | 87 | 81 | 86 | 89 | 81 | 86 | 91 |
| | 118 | 103 | 108 | 113 | 95 | 103 | 112 | 93 | 108 | 102 |
| Mean | 100 | | 97 | 100 | 88 | 95 | 101 | 87 | 97 | 96 |
| sd | 14 | | 16 | 19 | 10 | 13 | 16 | 9 | 16 | 8 |
| Morph. obs. | + | | + | + | + | + | + | + | + | + |
| Naringenin | | | | | | | | | | |
| Viability (%) | 95 | 96 | 96 | 95 | 93 | 95 | 89 | 85 | 76 | 48 |
| | 104 | 105 | 95 | 92 | 91 | 95 | 94 | 94 | 74 | 47 |
| Mean | 100 | | 95 | 93 | 92 | 95 | 92 | 89 | 75 | 47 |
| sd | 5 | | 1 | 2 | 1 | 0 | 4 | 6 | 2 | 1 |
| Morph. obs. | + | | + | + | + | + | + | + | +/−,* | +/−,* |
| Nar-5-Rha | | | | | | | | | | |
| Viability (%) | 96 | 99 | 91 | 92 | 85 | 94 | 92 | 78 | 82 | 79 |
| | 101 | 104 | 111 | 93 | 88 | 100 | 98 | 91 | 90 | 87 |

| | | | | | | | | | |
|---|---|---|---|---|---|---|---|---|---|
| Mean | 100 | 101 | 93 | 86 | 97 | 95 | 84 | 86 | 83 |
| sd | 3 | 14 | 1 | 2 | 4 | 4 | 9 | 6 | 6 |
| Morph. obs. | + | + | + | + | + | + | + | + | +/− |

Cytotoxicity measurements on monolayer cultures of NHEK demonstrated a better compatibility of the 5-O-α-L-rhamnosides versus their flavonoid aglycons at elevated concentration. Up to 100 μM no consistent differences were observed (FIG. 10). However, at 250 μM concentration of the aglycons hesperetin and naringenin the viability of NHEK was decreased to about 50% while the mitochondrial activity of NHEK treated with the corresponding 5-O-α-L-rhamnosides was still unaffected compared to lower concentrations. In particular these results were unexpected as the solubility of flavonoid aglycons generally is below 100 μM in aqueous phases while that of glycosidic derivatives is above 250 μM. These data clearly demonstrated that the 5-O-α-L-rhamnosides were less toxic to the normal human keratinocytes.

### Example D-2: Anti-Inflammatory Properties

### Anti-Inflammatory Potential

NHEK were pre-incubated for 24 h with the test compounds. The medium was replaced with the NHEK culture medium containing the inflammatory inducers (PMA or Poly I:C) and incubated for another 24 hours. Positive and negative controls ran in parallel. At the endpoint the culture supernatants were quantified of secreted IL-8, PGE2 and TNF-α, by means of ELISA.

### Anti-Inflammatory Effects of 5-O-Rhamnosides in NHEK Cell Cultures

TABLE B3
Inhibition of 5-O-rhamnosides on Cytokine release in human keratinocytes (NHEK)

| | | | | | | | % stim. | | | | | |
|---|---|---|---|---|---|---|---|---|---|---|---|---|
| Compound | | | Cytokine [pg/mL] | | | | control | | | Inhibition | | |
| Conc. | Stimulation | | Type | | Mean | sd | % | sd | p⁽¹⁾ | % | sd | p⁽¹⁾ |
| Non- | Control | | | 96 | 126 | 18 | 8 | 1 | *** | 100 | 1 | *** |
| stimulat | | | | 157 | | | | | | | | |
| | | | | 127 | | | | | | | | |
| Stimulated | Control | | | 1846 | 1569 | 141 | 100 | 9 | — | 0 | 10 | — |
| conditions: | | | | 1480 | | | | | | | | |
| PMA - 1 μg/ml | | | | 1381 | | | | | | | | |
| | Indomethacin | | | 39 | 39 | 0 | 2 | 0 | *** | 106 | 0 | *** |
| | 10⁻⁶M | | | 39 | | | | | | | | |
| | | | | 39 | | | | | | | | |
| | Dexamethasone | | | 1318 | 1437 | 168 | 92 | 11 | — | 9 | 12 | — |
| | 10⁻⁶M | | | 1556 | | | | | | | | |
| | HESR1 | PMA | PGE₂ | 582 | 507 | 107 | 32 | 7 | — | 74 | 7 | — |
| | (HES-5- | | | 431 | | | | | | | | |
| | Rha) | | IL-8 | 3242 | 2843 | 564 | 98 | 19 | — | 34 | 17 | |
| | 100 μM | | | 2445 | | | | | | | | |
| | | poly(I:C) | IL-8 | 2617 | 2793 | 250 | 76 | 7 | | 24 | 7 | |
| | | | | 2970 | | | | | | | | |
| | | | TNFα | 416 | 423 | 9 | 75 | 2 | | 26 | 2 | |
| | | | | 429 | | | | | | | | |
| | NR1 | PMA | PGE₂ | 851 | 1271 | 594 | 81 | 38 | — | 21 | 41 | — |
| | (N-5- | | | 1691 | | | | | | | | |
| | Rha) | | IL-8 | 2572 | 2564 | 12 | 88 | 0 | — | 12 | 0 | — |
| | 100 μM | | | 2555 | | | | | | | | |
| | | poly(I:C) | IL-8 | 3055 | 3154 | 140 | 86 | 4 | | 14 | 4 | |
| | | | | 3253 | | | | | | | | |
| | | | TNFα | 516 | 516 | 0 | 92 | 0 | | 8 | 0 | |
| | | | | 516 | | | | | | | | |

Compared to control experiments the 5-O-rhamnosides showed anti-inflammatory activities on human keratinocytes concerning three different inflammation markers IL-8, TNFα, and PGE2 under inflammatory stimuli (PMA, poly(I:C)). Especially, the activity of HESR1 on PGE2 was remarkable with a 74% inhibition. An anti-inflammatory activity is well documented for flavonoid derivatives. And several authors reported their action via COX, NFκB, and MAPK pathways (Biesalski (2007) Curr Opin Clin Nutr Metab Care 10(6):724-728, Santangelo (2007) Ann Ist Super Sanita 43(4): 394-405). However, the exceptional water solubility of flavonoid-5-O-rhamnosides disclosed here allows much higher intracellular concentrations of these compounds than achievable with their rarely soluble aglycon counterparts. The aglycon solubilities are mostly below their effective concentration. Thus, the invention enables higher efficacy for anti-inflammatory purposes.

Among many other regulatory activities TNFα also is a potent inhibitor of hair follicle growth (Lim (2003) Korean J Dermatology 41: 445-450). Thus, TNFα inhibiting compounds contribute to maintain normal healthy hair growth or even stimulate it.

### Example D-3: Antioxidative Properties

### Antioxidative Effects of 5-O-Rhamnosides in NHEK Cell Cultures

Pre-incubated NHEK were incubated with the test compound for 24 h. Then the specific fluorescence probe for the measurement of hydrogen peroxide (DHR) or lipid peroxides (C₁₁-fluor) was added and incubated for 45 min. Irradiation occurred with in H₂O₂ determination UVB at 180 mJ/cm² (+UVA at 2839 mJ/cm²) or UVB at 240 mJ/cm² (+UVA at 3538 mJ/cm²) in lipid peroxide, respectively, using a SOL500 Sun Simulator lamp. After irradiation the cells were post-incubated for 30 min before flow-cytometry analysis.

TABLE B4
Protection of 5-O-rhamnosides against UV-induced H₂O₂ stress in NHEK cells

| | | | | | | % irradiated | | | | | |
|---|---|---|---|---|---|---|---|---|---|---|---|
| Test | | | H₂O₂ (AU) | | | control | | | Protection | | |
| compound | | Concentration | (DHR GMFI) | Mean | sd | % | sd | p⁽¹⁾ | % | sd | p⁽¹⁾ |
| Non-Irradiated | No DHR | — | 9 | 8.77 | 0 | — | — | — | — | — | — |
| condition | probe | | 8 | | | | | | | | |
| | | | 9 | | | | | | | | |
| | Control | | 311 | 316.33 | 3 | 17 | 0 | ** | 100 | 0 | ** |
| | | | 319 | | | | | | | | |
| | | | 319 | | | | | | | | |
| Irradiated | Control | | 1770 | 1846.83 | 209 | 100 | 11 | — | 0 | 14 | — |
| conditions: | | | 1307 | | | | | | | | |
| 180 mJ/cm² UVB | | | 2388 | | | | | | | | |
| (2839 mJ/cm² UVA) | | | 1182 | | | | | | | | |
| | | | 2169 | | | | | | | | |
| | | | 2265 | | | | | | | | |
| | BHA | 100 μM | 740 | 776 | 29 | 42 | 2 | * | 70 | 2 | * |
| | | | 834 | | | | | | | | |
| | | | 754 | | | | | | | | |
| | Vit. E | 50 μM | 628 | 655 | 17 | 35 | 1 | ** | 78 | 1 | ** |
| | | | 650 | | | | | | | | |
| | | | 687 | | | | | | | | |
| | HESR1 | 100 μM | 1046 | 1152 | 150 | 62 | 8 | — | 45 | 10 | |
| | | | 1258 | | | | | | | | |
| | NR1 | 100 μM | 2531 | 2516.5 | 21 | 136 | 1 | — | −44 | 1 | |
| | | | 2502 | | | | | | | | |

TABLE B5
Protection of 5-O-rhamnosides against UV-induced lipid peroxide in NHEK cells

| | | | | | | | % Irradiated | | | | | |
|---|---|---|---|---|---|---|---|---|---|---|---|---|
| Test | | | C11-fluor (AU) | | | | control | | | Protection | | |
| compound | | Concentration | GMFI | 1/GMFI | Mean | sd | % | sd | p⁽¹⁾ | % | sd | p⁽¹⁾ |
| Non- | No C11- | — | 3 | 3.1E−01 | 3.1E−01 | 1.1E−02 | — | — | — | — | — | — |
| Irradiated | fluor | | 3 | 3.0E−01 | | | | | | | | |
| condition | probe | | 3 | 3.3E−01 | | | | | | | | |
| | Control | — | 9049 | 1.1E−04 | 1.1E−04 | 7.6E−06 | 23 | 2 | *** | 100 | 2 | *** |
| | | | 10874 | 9.2E−05 | | | | | | | | |
| | | | 8504 | 1.2E−04 | | | | | | | | |
| Irradiated | Control | | 2273 | 4.4E−04 | 4.6E−04 | 1.2E−05 | 100 | 3 | — | 0 | 3 | — |
| conditions: | | | 2072 | 4.8E−04 | | | | | | | | |
| 240 mJ/cm² | | | 2166 | 4.6E−04 | | | | | | | | |
| UVB | BHT | 50 μM | 3139 | 3.2E−04 | 3.3E−04 | 8.5E−06 | 72 | 2 | | 37 | 2 | *** |
| (3538 mJ/cm² UVA) | | | 3047 | 3.3E−04 | | | | | | | | |
| | | | 2877 | 3.5E−04 | | | | | | | | |
| | HESR1 | 100 μM | 1671 | 6.0E−04 | 6.4E−04 | 6.3E−05 | 99 | 10 | — | 1 | 12 | |
| | | | 1455 | 6.9E−04 | | | | | | | | |
| | NR1 | 100 μM | 2414 | 4.1E−04 | 4.3E−04 | 2.1E−05 | 93 | 4 | — | 9 | 6 | — |
| | | | 2255 | 4.4E−04 | | | | | | | | |

An anti-oxidative function of the tested flavonoid-5-O-rhamnosides could be observed for HESR1 on mitochondrially produced hydrogen peroxides species and for NR1 on lipid peroxides, respectively. However, it is argued that these parameters are influenced also by different intracellular metabolites and factors, e.g. gluthation. Hence, interpretation of anti-oxidative response often is difficult to address to a single determinant.

### Example D-4: Stimulating Properties of 5-O-rhamnosides

Tests were performed with normal human dermal fibroblast cultures at the 8^{th} passage. Cells were grown in DMEM supplemented with glutamine at 2 mM, penicillin at 50 U/mL and streptomycin (50 μg/mL) and 10% of fetal calf serum (FCS) at 37° C. in a 5% CO₂ atmosphere.

### Stimulation of Flavonoid-5-O-rhamnosides on Syntheses of Procollagen I, Release of VEGF, and Fibronectin Production in NHDF Cells

Fibroblasts were cultured for 24 hours before the cells were incubated with the test compounds for further 72 hours. After the incubation the culture supernatants were collected in order to measure the released quantities of procollagen I, VEGF, and fibronectin by means of ELISA. Reference test compounds were vitamin C (procollagen I), PMA (VEGF), and TGF-β (fibronectin).

TABLE B6
Stimulation of 5-O-rhamnosides on procollagen I synthesis in NHDF cells

| | | Basic data | | | | | | Normalized data | | |
|---|---|---|---|---|---|---|---|---|---|---|
| Treatment | | Procollagen I | | | % | | | % | | |
| Compound | Conc. | (ng/ml) | Mean | sd | Control | sd | p⁽¹⁾ | Stimulation | sd | p⁽¹⁾ |
| Control | — | 1893 | 1667 | 122 | 100 | 7 | — | 0 | 7 | — |
| | | 1473 | | | | | | | | |
| | | 1637 | | | | | | | | |
| Vitamin C | 20 μg/ml | 4739 | 5272 | 323 | 316 | 19 | *** | 216 | 19 | *** |
| | | 5854 | | | | | | | | |
| | | 5225 | | | | | | | | |
| NR1 | 100 μM | 1334 | 1097 | 335 | 66 | 20 | — | −34 | 20 | |
| | | 860 | | | | | | | | |
| HESR1 | 100 μM | 1929 | 1968 | 55 | 118 | 3 | — | 18 | 3 | |
| | | 2007 | | | | | | | | |

TABLE B7
Stimulation of 5-O-rhamnosides on VEGF release in NHDF cells

| | | Basic data | | | | | | Normalized data | | |
|---|---|---|---|---|---|---|---|---|---|---|
| Treatment | | VEGF | Mean VEGF | | % | sd | | % | sd | |
| Compound | Conc. | (pg/ml) | (pg/ml) | sd | Control | (%) | p⁽¹⁾ | Stimulation | (%) | p⁽¹⁾ |
| Control | — | 83 | 72 | 6 | 100 | 9 | — | 0 | 9 | — |
| | | 73 | | | | | | | | |
| | | 61 | | | | | | | | |
| PMA | 1 μg/ml | 150 | 148 | 3 | 205 | 4 | *** | 105 | 4 | *** |
| | | 150 | | | | | | | | |
| | | 143 | | | | | | | | |
| NR1 | 100 μM | 90 | 92 | 3 | 128 | 4 | — | 28 | 4 | |
| | | 94 | | | | | | | | |
| HESR1 | 100 μM | 70 | 73 | 5 | 101 | 6 | — | 1 | 6 | |
| | | 76 | | | | | | | | |

TABLE B8
Stimulation of 5-O-rhamnosides on fibronectin synthesis in NHDF cells

| | | Basic data | | | | | | Normalized data | | |
|---|---|---|---|---|---|---|---|---|---|---|
| Treatment | | Fibronectin | Mean | | % | sd | | % | sd | |
| Compound | Conc. | (ng/ml) | (ng/ml) | sd | Control | (%) | p⁽¹⁾ | Stimulation | (%) | p⁽¹⁾ |
| Control | — | 6017 | 6108 | 86 | 100 | 1 | — | 0 | 1 | — |
| | | 6281 | | | | | | | | |
| | | 6027 | | | | | | | | |
| TGF-β | 10 ng/ml | 10870 | #### | 95 | 181 | 2 | *** | 81 | 2 | *** |
| | | 11178 | | | | | | | | |
| | | 11128 | | | | | | | | |
| NR1 | 100 μM | 6833 | 7326 | 698 | 120 | 11 | — | 20 | 11 | |
| | | 7820 | | | | | | | | |
| HESR1 | 100 μM | 5843 | 5853 | 14 | 96 | 0 | — | −4 | 0 | |
| | | 5864 | | | | | | | | |

Results demonstrated that flavonoid-5-O-rhamnosides can positively affect extracellular matrix components. HESR1 stimulated procollagen I synthesis in NHDF by about 20% at 100 μM. NR1 at 100 μM had a stimulating effect on fibronectin synthesis with an increase of 20% in NHDF. Both polymers are well known to be important extracellular tissue stabilization factors in human skin. Hence substances promoting collagen synthesis or fibronectin synthesis support a firm skin, reduce wrinkles and diminish skin aging. VEGF release was also stimulated approx. 30% by NR1 indicating angiogenic properties of flavonoid-5-O-rhamnosides. Moderate elevation levels of VEGF are known to positively influence hair and skin nourishment through vascularization and thus promote e.g. hair growth (Yano (2001) J Clin Invest 107:409-417, KR101629503B1). Also, Fibronectin was described to be a promoting factor on human hair growth as stated in US 2011/0123481 A1. Hence, NR1 stimulates hair growth by stimulating the release of VEGF as well as the synthesis of fibronectin in normal human fibroblasts.

### Stimulation of Flavonoid-5-O-rhamnosides on MMP-1 Release in UVA-Irradiated NHDF

Human fibroblasts were cultured for 24 hours before the cells were pre-incubated with the test or reference compounds (dexamethasone) for another 24 hours. The medium was replaced by the irradiation medium (EBSS, CaCl₂) 0.264 g/L, MgClSO₄ 0.2 g/L) containing the test compounds) and cells were irradiated with UVA (15 J/cm²). The irradiation medium was replaced by culture medium including again the test compounds incubated for 48 hours. After incubation the quantity of matrix metallopeptidase 1 (MMP-1) in the culture supernatant was measured using an ELISA kit.

TABLE B10
Stimulation of 5-O-rhamnosides on UV-induced MMP-1 release in NHDF cells

| | | | Basic data | | | | | | | | |
|---|---|---|---|---|---|---|---|---|---|---|---|
| | | | | Mean | | % | | | Normalized data | | |
| Treatment | | | MMP-1 | MMP-1 | | Irradiated | sd | | % | sd | |
| Test compound | | Conc. | (ng/ml) | (ng/ml) | sd | control | (%) | p⁽¹⁾ | Protection | (%) | p⁽¹⁾ |
| Non- | Control | — | 28.1 | 25.5 | 1.6 | 36 | 2 | ** | 100 | 4 | ** |
| Irradiated | | | 26.1 | | | | | | | | |
| | | | 22.5 | | | | | | | | |
| Irradiated conditions: | Control | — | 83.7 | 71.0 | 7.1 | 100 | 10 | — | 0 | 16 | — |
| 15 J/cm² UVA | | | 59.1 | | | | | | | | |
| | | | 70.3 | | | | | | | | |
| | Dexamethasone | 10⁻⁷M | 2.5 | 2.9 | 0.2 | 4 | 0 | *** | 150 | 0 | *** |
| | | | 3.1 | | | | | | | | |
| | | | 3.2 | | | | | | | | |
| | NR1 | 100 μM | 211.7 | 240.3 | 40.3 | 338 | 57 | — | −372 | 89 | |
| | | | 268.8 | | | | | | | | |
| | HESR1 | 100 μM | 87.0 | 82.2 | 6.8 | 116 | 10 | — | −25 | 15 | |
| | | | 77.4 | | | | | | | | |

Flavonoid-5-O-rhamnosides showed high activities on MMP-1 levels in NHDF. NR1 caused a dramatic upregulation of MMP-1 biosynthesis nearly 4-fold in UV-irradiated conditions.

MMP-1 also known as interstitial collagenase is responsible for collagen degradation in human tissues. Here, MMP-1 plays important roles in pathogenic arthritic diseases but was also correlated with cancer via metastasis and tumorigenesis (Vincenti (2002) Arthritis Res 4:157-164, Henckels (2013) F₁₀₀₀Research 2:229). Additionally, MMP-1 activity is important in early stages of wound healing (Caley (2015) Adv Wound Care 4: 225-234). Thus, MMP-1 regulating compounds can be useful in novel wound care therapies, especially if they possess anti-inflammatory and VEGF activities as stated above.

NR1 even enables novel therapies against arthritic diseases via novel biological regulatory targets. For example, MMP-1 expression is regulated via global MAPK or NFκB pathways (Vincenti and Brinckerhoff 2002, Arthritis Research 4(3):157-164). Since flavonoid-5-O-rhamnosides are disclosed here to possess anti-inflammatory activities and reduce IL-8, TNFα, and PGE-2 release, pathways that are also regulated by MAPK and NFκB. Thus, one could speculate that MMP-1 stimulation by flavonoid-5-O-rhamnosides is due to another, unknown pathway that might be addressed by novel pharmaceuticals to fight arthritic disease.

Current dermocosmetic concepts to reduce skin wrinkles address the activity of collagenase. Next to collagenase inhibition one contrary concept is to support its activity. In this concept misfolded collagene fibres that solidify wrinkles within the tissue are degraded by the action of collagenases. Simultaneously, new collagene has to be synthesized by the tissue to rebuild skin firmness. In this concept, the disclosed flavonoid-5-O-rhamnosides combine ideal activities as they show stimulating activity of procollagen and MMP-1.

Finally, MMP-1 upregulating flavonoid-5-O-rhamnosides serve as drugs in local therapeutics to fight abnormal collagene syndroms like Dupuytren's contracture.

### Example D-5: Modulation of Transcriptional Regulators by Flavonoid-5-O-Rhamnosides

### NF-κB Activity in Fibroblasts

NIH3T3-KBF-Luc cells were stably transfected with the KBF-Luc plasmid (Sancho (2003) Mol Pharmacol 63:429-438), which contains three copies of NF-κB binding site (from major histocompatibility complex promoter), fused to a minimal simian virus 40 promoter driving the luciferase gene. Cells (1×10⁴ for NIH3T3-KBF-Luc) were seeded the day before the assay on 96-well plate. Then the cells were treated with the test substances for 15 min and then stimulated with 30 ng/ml of TNFα. After 6 h, the cells were washed twice with PBS and lysed in 50 μl lysis buffer containing 25 mM Tris-phosphate (pH 7.8), 8 mM MgCl₂, 1 mM DTT, 1% Triton X-100, and 7% glycerol during 15 min at RT in a horizontal shaker. Luciferase activity was measured using a GloMax 96 microplate luminometer (Promega) following the instructions of the luciferase assay kit (Promega, Madison, Wis., USA). The RLU was calculated and the results expressed as percentage of inhibition of NF-κB activity induced by TNFα (100% activation) (tables B10.1-B10.3). The experiments for each concentration of the test items were done in triplicate wells.

TABLE B10.1
Influence of 5-O-rhamnosides on NF-κB activity in NIH3T3 cells

| | | | | | | RLU | % |
|---|---|---|---|---|---|---|---|
| | | RLU 1 | RLU 2 | RLU 3 | MEAN | specific | Activation |
| | Control | 38240 | 38870 | 34680 | 37263 | 0 | 0 |
| | TNFα 30 ng/ml | 115870 | 120220 | 121040 | 119043 | 81780 | 100.0 |
| +30 ng/ml TNFα | HESR1 10 μM | 186120 | 181040 | 182280 | 183147 | 145883 | 178.4 |
| | HESR1 25 μM | 218940 | 216580 | 213320 | 216280 | 179017 | 218.9 |
| | NR1 10 μM | 134540 | 126580 | 130240 | 130453 | 93190 | 114.0 |
| | NR1 25 μM | 151080 | 151840 | 143870 | 148930 | 111667 | 136.5 |
| | Chrysin 10 μM | 301630 | 274240 | 303950 | 293273 | 256010 | 313.0 |
| | Chrysin 25 μM | 273410 | 272580 | 285980 | 277323 | 240060 | 293.5 |

TABLE B10.2
Influence of 5-O-rhamnosides on NF-κB activity in NIH3T3 cells

| | | | | | | RLU | % |
|---|---|---|---|---|---|---|---|
| | | RLU 1 | RLU 2 | RLU 3 | MEAN | specific | Activation |
| | Control | 23060 | 23330 | 23700 | 23363 | 0 | 0 |
| | TNFα 30 ng/ml | 144940 | 156140 | 160200 | 153760 | 130397 | 100.0 |
| +30 ng/ml TNFα | CR1 10 μM | 157870 | 169000 | 173010 | 166627 | 143263 | 109.9 |
| | CR1 25 μM | 175140 | 183630 | 183960 | 180910 | 157547 | 120.8 |
| | CR2 10 μM | 156600 | 160140 | 151070 | 155937 | 132573 | 101.7 |
| | CR2 25 μM | 170390 | 179220 | 163490 | 171033 | 147670 | 113.2 |
| | Diosmetin 10 μM | 398660 | 411390 | 412940 | 407663 | 384300 | 294.7 |
| | Diosmetin 25 μM | 448530 | 452660 | 451610 | 450933 | 427570 | 327.9 |
| | DR2 10 μM | 211150 | 215320 | 213260 | 213243 | 189880 | 145.6 |
| | DR2 25 μM | 245900 | 241550 | 234880 | 240777 | 217413 | 166.7 |
| | Biochanin A 10 μM | 588070 | 586440 | 579220 | 584577 | 561213 | 430.4 |
| | Biochanin A 25 μM | 570360 | 573190 | 594510 | 579353 | 555990 | 426.4 |
| | BR1 10 μM | 259120 | 247590 | 229500 | 245403 | 222040 | 170.3 |
| | BR1 25 μM | 211660 | 208010 | 203720 | 207797 | 184433 | 141.4 |
| | BR2 10 μM | 205410 | 202640 | 202940 | 203663 | 180300 | 138.3 |
| | BR2 25 μM | 237390 | 235850 | 235350 | 236197 | 212833 | 163.2 |

TABLE B10.3
Influence of 5-O-rhamnosides on NF-κB activity in NIH3T3 cells

| | | | | | | RLU | % |
|---|---|---|---|---|---|---|---|
| | | RLU 1 | RLU 2 | RLU 3 | MEAN | specific | Activation |
| | Control | 32200 | 33240 | 33100 | 32847 | 0 | 0 |
| | TNFα 30 ng/ml | 179150 | 179270 | 184270 | 180897 | 148050 | 100.0 |
| +30 ng/ml | Silibinin 10 μM | 249050 | 238550 | 231180 | 239593 | 206747 | 139.6 |
| TNFα | Silibinin 25 μM | 212420 | 210050 | 200660 | 207710 | 174863 | 118.1 |
| | SR1 10 μM | 269710 | 262180 | 254090 | 261993 | 229147 | 154.8 |
| | SR1 25 μM | 174940 | 171280 | 171730 | 172650 | 139803 | 94.4 |

It was reported that NF-κB activity is reduced by many flavonoids (Prasad (2010) Planta Med 76: 1044-1063). Chrysin was reported to inhibit NF-κB activity through the inhibition of IκBα phosphorylation (Romier(2008) Brit J Nutr 100: 542-551). However, when NIH3T3-KBF-Luc cells were stimulated with TNFα the activity of NF-κB was generally co-stimulated by flavonoids and their 5-O-rhamnosides at 10 μM and 25 μM, respectively.

### STAT3 Activity

HeLa-STAT3-luc cells were stably transfected with the plasmid 4×M67 pTATA TK-Luc. Cells (20×10³ cells/ml) were seeded 96-well plate the day before the assay. Then the cells were treated with the test substances for 15 mM and then stimulated with IFN-γ 25 IU/ml. After 6 h, the cells were washed twice with PBS and lysed in 50₁11 lysis buffer containing 25 mM Tris-phosphate (pH 7.8), 8 mM MgCl₂, 1 mM DTT, 1% Triton X-100, and 7% glycerol during 15 mM at RT in a horizontal shaker. Luciferase activity was measured using GloMax 96 microplate luminometer (Promega) following the instructions of the luciferase assay kit (Promega, Madison, Wis., USA). The RLU was calculated and the results were expressed as percentage of inhibition of STAT3 activity induced by IFN-γ (100% activation) (tables B11.1-B11.3). The experiments for each concentration of the test items were done in triplicate wells.

TABLE B11.1
STAT3 activation by flavonoids and their 5-O-rhamnosides in HeLa cells

| | | | | | | RLU | |
|---|---|---|---|---|---|---|---|
| | | RLU 1 | RLU 2 | RLU 3 | MEAN | specific | % Activation |
| | Control | 2060 | 2067 | 1895 | 2007 | 0 | 0 |
| | IFNγ 25 U/ml | 12482 | 15099 | 15993 | 14525 | 12517 | 100 |
| +IFNγ 25 U/ml | HESR1 25 μM | 13396 | 12243 | 12859 | 12833 | 10825 | 86.48 |
| | HESR1 50 μM | 14303 | 13124 | 11985 | 13137 | 11130 | 88.92 |
| | NR1 25 μM | 10925 | 8301 | 8752 | 9326 | 7319 | 58.47 |
| | NR1 50 μM | 18272 | 6426 | 7599 | 10766 | 8758 | 69.97 |
| | Chrysin 25 μM | 28031 | 22367 | 17504 | 22634 | 20627 | 164.78 |
| | Chrysin 50 μM | 27912 | 3531 | 16304 | 15916 | 13908 | 111.11 |
| | C57dR 25 μM | 11316 | 1954 | 8493 | 7254 | 5247 | 41.92 |
| | C57dR 50 μM | 9196 | 2358 | 6307 | 5954 | 3946 | 31.53 |
| | C5R 25 μM | 7897 | 2398 | 5326 | 5207 | 3200 | 25.56 |
| | C5R 50 μM | 6897 | 7665 | 10507 | 8356 | 6349 | 50.72 |
| | Diosmetin 25 μM | 16337 | 14303 | 17066 | 15902 | 13895 | 111.00 |
| | Diosmetin 50 μM | 9189 | 7751 | 7857 | 8266 | 6258 | 50.00 |
| | D5R 25 μM | 12137 | 10269 | 9275 | 10560 | 8553 | 68.33 |
| | D5R 50 μM | 13005 | 10547 | 10143 | 11232 | 9224 | 73.69 |

TABLE B11.2
STAT3 activation by flavonoids and their 5-O-rhamnosides in HeLa cells

| | | | | | | RLU | |
|---|---|---|---|---|---|---|---|
| | | RLU 1 | RLU 2 | RLU 3 | MEAN | specific | % Activation |
| | Control | 1875 | 1815 | 1815 | 1835 | 0 | 0 |
| | IFNγ 25 U/ml | 9659 | 9851 | 10116 | 9875 | 8040 | 100 |
| +IFNγ 25 U/ml | Biochanin A 25 μM | 9732 | 9023 | 8911 | 9222 | 7387 | 91.87 |
| | Biochanin A 50 μM | 6804 | 12097 | 11786 | 10229 | 8394 | 104.40 |
| | BR1 25 μM | 8162 | 12819 | 11157 | 10713 | 8878 | 110.41 |
| | BR1 50 μM | 12336 | 11620 | 12104 | 12020 | 10185 | 126.67 |
| | BR2 25 μM | 11157 | 10163 | 10660 | 10660 | 8825 | 109.76 |
| | BR2 50 μM | 7983 | 9023 | 11110 | 9372 | 7537 | 93.74 |
| | Silibinin 25 μMI | 12389 | 11170 | 11210 | 11590 | 9755 | 121.32 |
| | Silibinin 50 μM | 12157 | 11885 | 10540 | 11527 | 9692 | 120.55 |

TABLE B11.3
STAT3 activation by flavonoids and their 5-O-rhamnosides in HeLa cells

| | | | | | RLU | % Acti- |
|---|---|---|---|---|---|---|
| | RLU 1 | RLU 2 | RLU 3 | MEAN | specific | vation |
| Control | 2312 | 2233 | 2173 | 2239 | 0 | 0 |
| IFNγ 25 U/ml | 11375 | 10852 | 11269 | 11165 | 9158 | 100 |
| SR1 25 μM + | 9507 | 11653 | 10203 | 10454 | 8447 | 92.24 |
| IFNγ 25 U/ml | | | | | | |
| SR1 50 μM + | 10090 | 11355 | 10938 | 10794 | 8787 | 95.95 |
| IFNγ 25 U/ml | | | | | | |

STAT3 is a transcriptional factor of many genes related to epidermal homeostasis. Its activity has effects on tissue repair and injury healing but also is inhibiting on hair follicle regeneration (Liang (2012) J Neurosci32:10662-10673). STAT3 activity may even promote melanoma and increases expression of genes linked to cancer and metastasis (Cao(2016) Sci. Rep. 6, 21731).

### Example D-6: Alteration of Glucose Uptake into Cells by Flavonoid 5-O-Rhamnosides

### Determination of Glucose Uptake in Keratinocytes

HaCaT cells (5×10⁴) were seeded in 96-well black plates and incubated for 24 h. Then, medium was removed and the cells cultivated in OptiMEM, labeled with 50 μM 2-NBDG (2-[N-(7-nitrobenz-2-oxa-1,3-diazol-4-yl) amino]-2-deoxy-D-glucose and treated with the test substances or the positive control, Rosiglitazone, for 24 h. Medium was removed and the wells were carefully washed with PBS and incubated in PBS (100 μl/well). Finally the fluorescence was measured using the Incucyte FLR software, the data were analyzed by the total green object integrated intensity (GCU×μm2×Well) of the imaging system IncuCyte HD (Essen BioScience). The fluorescence of Rosiglitazone is taken as 100% of glucose uptake, and the glucose uptake was calculated as (% Glucose uptake)=100(T−B)/(R−B), where T (treated) is the fluorescence of test substance-treated cells, B (Basal) is the fluorescence of 2-NBDG cells and P (Positive control) is the fluorescence of cells treated with Rosiglitazone. Results of triplicate measurements are given in tables B12.1 and B12.2.

TABLE B12.1
Influence of flavonoid 5-O-rhamnosides on Glucose uptake in HaCaT cells

| | | | | | | | % |
|---|---|---|---|---|---|---|---|
| | | | | | | RFU | Glucose |
| | | Measure 1 | Measure 2 | Measure 3 | Mean | specific | uptake |
| | Control | 8945 | 6910 | 3086 | 6314 | 0 | 0.0 |
| | 2NBDG 50 μM | 176818 | 359765 | 312467 | 283017 | 276703 | 0.0 |
| +2NBDG 50 μM | Rosiglitazone | 776381 | 707003 | 1141924 | 875103 | 868789 | 100.0 |
| | 80 μM | | | | | | |
| | HESR1 25 μM | 756943 | 549324 | 384251 | 563506 | 557192 | 64.1 |
| | HESR1 50 μM | 501977 | 642949 | 529620 | 558182 | 551868 | 63.5 |
| | NR1 25 μM | 493970 | 1160754 | 649291 | 768005 | 761691 | 87.7 |
| | NR1 50 μM | 278134 | 256310 | 257198 | 263881 | 257567 | 29.6 |
| | CR1 25 μM | 291406 | 358114 | 628963 | 426161 | 419847 | 48.3 |
| | CR1 50 μM | 619992 | 595330 | 174412 | 463245 | 456931 | 52.6 |
| | CR2 25 μM | 427937 | 431593 | 390512 | 416681 | 410367 | 47.2 |
| | CR2 50 μM | 771478 | 1100390 | 923151 | 931673 | 925359 | 106.5 |
| | DR2 25 μM | 632398 | 940240 | 197738 | 590125 | 583811 | 67.2 |
| | DR2 50 μM | 2958363 | 1297231 | 2493030 | 2249541 | 2243227 | 258.2 |

TABLE B12.2
Influence of flavonoid 5-O-rhamnosides on Glucose uptake in HaCaT cells

| | | | | | | | % |
|---|---|---|---|---|---|---|---|
| | | | | | | RFU | Glucose |
| | | Measure 1 | Measure 2 | Measure 3 | Mean | specific | uptake |
| | Control | 44637 | 49871 | 4750 | 33086 | 0 | 0.0 |
| | 2NBDG 50 μM | 492141 | 470496 | 873235 | 611957 | 578871 | 0.0 |
| +2NBDG 50 μM | Rosiglitazone | 923011 | 1440455 | 1584421 | 1315962 | 1282877 | 100.0 |
| | 80 μM | | | | | | |
| | BR1 25 μM | 730362 | 661244 | 400131 | 597246 | 564160 | 44.0 |
| | BR1 50 μM | 899548 | 626443 | 743535 | 756509 | 723423 | 56.4 |
| | BR2 25 μM | 998132 | 1149619 | 935073 | 1027608 | 994522 | 77.5 |
| | BR2 50 μM | 1657600 | 1788604 | 1619334 | 1688513 | 1655427 | 129.0 |
| | SR1 25 μM | 579565 | 3067153 | 4212718 | 2619812 | 2586726 | 201.6 |
| | SR1 50 μM | 2064420 | 3541782 | 2654102 | 2753435 | 2720349 | 212.1 |

1. A method for the production of rhamnosylated flavonoids, the method comprising
(a) contacting/incubating a glycosyl transferase with a flavonoid; and
(b) obtaining a rhamnosylated flavonoid,
wherein the glycosyl transferase
(a) comprises the amino acid sequence of SEQ ID NO: 1;
(b) comprises amino acid sequences having at least 80% sequence identity with SEQ ID NOs: 3, 5, 7, 9, 11, 13, 15, 17, 19, 21, 23, 25, 56, 58, 61;
(c) is encoded by a polynucleotide comprising the nucleic acid sequences of SEQ ID NOs: 2, 4, 6, 8, 10, 12, 14, 16, 18, 20, 22, 24, 26, 27, 28, 29, 30, 31, 32, 33, 34, 35, 36 37, 38, 57, 59, 60, 62, or 63;
(d) is encoded by a polynucleotide having at least 80% sequence identity with SEQ ID NOs: 2, 4, 6, 8, 10, 12, 14, 16, 18, 20, 22, 24, 26, 27, 28, 29, 30, 31, 32, 33, 34, 35, 36 37, 38, 57, 59, 60, 62, or 63; or
(e) is encoded by a polynucleotide hybridizable under stringent conditions with a polynucleotide comprising SEQ ID NOs: 2, 4, 6, 8, 10, 12, 14, 16, 18, 20, 22, 24, 26, 27, 28, 29, 30, 31, 32, 33, 34, 35, 36 37, 38, 57, 59, 60, 62, or 63,
and
wherein the flavonoid is a compound or a solvate of the following Formula (I) 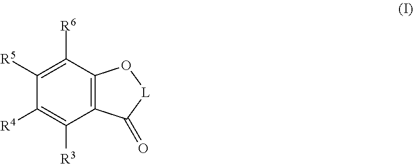
wherein: [symbol]
is a double bond or a single bond;
L is 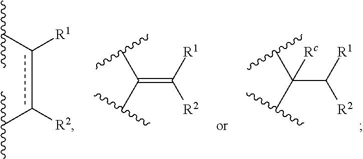
R¹ and R² are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; wherein R² is different from OH;
or R¹ and R² are joined together to form, together with the carbon atom(s) that they are attached to, a carbocyclic or heterocyclic ring being optionally substituted with one or more substituents R^{e}; wherein each R^{e} is independently selected from C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
R⁴, R⁵ and R⁶ are independently selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
or alternatively, R⁴ is selected from hydrogen, C₁₋₅; alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c}; and
R⁵ and R⁶ are joined together to form, together with the carbon atoms that they are attached to, a carbocyclic or heterocyclic ring being optionally substituted with one or more substituents R^{c};
or alternatively, R⁴ and R⁵ are joined together to form, together with the carbon atoms that they are attached to, a carbocyclic or heterocyclic ring being optionally substituted with one or more substituents R^{c}; and
R⁶ is selected from hydrogen, C₁₋₅ alkyl, C₂₋₅ alkenyl, C₂₋₅ alkynyl, heteroalkyl, cycloalkyl, heterocycloalkyl, aryl, heteroaryl, —R^{a}—R^{b}, —R^{a}—OR^{b}, —R^{a}—OR^{d}, —R^{a}—OR^{a}—OR^{b}, —R^{a}—OR^{a}—OR^{d}, —R^{a}—SR^{b}, —R^{a}—SR^{a}—SR^{b}, —R^{a}—NR^{b}R^{b}, —R^{a}-halogen, —R^{a}—(C₁₋₅ haloalkyl), —R^{a}—CN, —R^{a}—CO—R^{b}, —R^{a}—CO—O—R^{b}, —R^{a}—O—CO—R^{b}, —R^{a}—CO—NR^{b}R^{b}, —R^{a}—NR^{b}—CO—R^{b}, —R^{a}—SO₂—NR^{b}R^{b} and —R^{a}—NR^{b}—SO₂—R^{b}; wherein said alkyl, said alkenyl, said alkynyl, said heteroalkyl, said cycloalkyl, said heterocycloalkyl, said aryl and said heteroaryl are each optionally substituted with one or more groups R^{c};
each R^{a} is independently selected from a single bond, C₁₋₅ alkylene, C₂₋₅ alkenylene, arylene and heteroarylene; wherein said alkylene, said alkenylene, said arylene and said heteroarylene are each optionally substituted with one or more groups R^{c};
each R^{b} is independently selected from hydrogen, C₁₋₅ alkyl, C₂₋₅ alkenyl, C₂₋₅ alkynyl, heteroalkyl, cycloalkyl, heterocycloalkyl, aryl and heteroaryl; wherein said alkyl, said alkenyl, said alkynyl, said heteroalkyl, said cycloalkyl, said heterocycloalkyl, said aryl and said heteroaryl are each optionally substituted with one or more groups R^{c},
each R^{c} is independently selected from C₁₋₅ alkyl, C₂₋₅ alkenyl, C₂₋₅ alkynyl, —(C₀₋₃ alkylene)-OH, —(C₀₋₃ alkylene)-O—R^{d}, —(C₀₋₃ alkylene)-O(C₁₋₅ alkyl), —(C₀₋₃ alkylene)-O-aryl, —(C₀₋₃ alkylene)-O(C₁₋₅ alkylene)-OH, —(C₀₋₃ alkylene)-O(C₁₋₅ alkylene)-O—R^{d}, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S-aryl, —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-CN, —(C₀₋₃ alkylene)-CHO, alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl); wherein said alkyl, said alkenyl, said alkynyl and the alkyl or alkylene moieties comprised in any of the aforementioned groups R^{c} are each optionally substituted with one or more groups independently selected from halogen, —CF₃, —CN, —OH, —O—R^{d}, —O—C₁₋₄ alkyl and —S—C₁₋₄ alkyl;
each R^{d} is independently selected from a monosaccharide, a disaccharide and an oligosaccharide; and
R³ is rhamnoslyated by said method.

2. The method of claim 1, wherein the flavonoid is contacted/incubated with said glycosyl transferase at a final concentration above its solubility in aqueous solutions.

3. The method of claim 1, wherein the method further comprises a step of providing a host cell transformed with said glycosyl transferase.

4. The method of claim 3, wherein said host cell is incubated prior to contacting/incubating said host cell with a flavonoid.

5. The method of claim 3, wherein said host cell is Escherichia coli.

6. The method of claim 1, wherein contacting and/or incubating is/are done at a temperature from about 20° C. to about 37° C., preferably at a temperature from about 24° C. to about 30° C., and more preferably at a temperature of about 28° C.

7. The method of claim 1, wherein contacting/incubating is/are done at a pH of about 6.5 to about 8.5, preferably at a pH of about 7 to about 8, and more preferably at a pH of about 7.4.

8. The method of claim 1, wherein contacting/incubating is/are done at a concentration of dissolved oxygen (DO) of about 30% to about 50%.

9. The method of claim 1, wherein, when the concentration of dissolved oxygen is above about 50%, a nutrient is added, preferably wherein the nutrient is glucose, sucrose, maltose or glycerol.

10. The method of claim 1, wherein contacting/incubating is/are done in a complex nutrient medium.

11. The method of claim 1, wherein contacting/incubating is/are done in minimal medium.

12. The method of claim 3, wherein the method further comprises a step of harvesting said incubated host cell prior to contacting/incubating said host cell with a flavonoid.

13. The method of claim 12, wherein harvesting is done using a membrane filtration method, preferably a hollow fibre membrane device, or centrifugation.

14. The method of claim 12, wherein the method further comprises solubilization of the harvested host cell in a buffer prior to contacting/incubating said host cell with a flavonoid, preferably wherein the buffer is phosphate-buffered saline (PBS), preferably supplemented with a carbon and energy source, preferably glycerol, glucose, maltose, and/or sucrose, and growth additives, preferably vitamins including biotin and/or thiamin.

15. The method of claim 1, wherein the flavonoid is a flavanone, flavone, isoflavone, flavonol, flavanonol, chalcone, flavanol, anthocyanidine, aurone, flavan, chromene, chromone or xanthone.

16. The method of claim 1, wherein rhamnosylating is the addition of —O-(rhamnosyl) at position R³ of Formula (I) of claim 1, wherein said rhamnosyl is substituted at one or more of its —OH groups with one or more groups independently selected from C₁₋₅ alkyl, C₂₋₅ alkenyl, C₂₋₅ alkynyl, a monosaccharide, a disaccharide and an oligosaccharide.

17. The method of claim 1, wherein the flavonoid is contacted/incubated with said glycosyl transferase at a final concentration above about 200 μM.

18. The method of claim 1, wherein the flavonoid is contacted/incubated with said glycosyl transferase at a final concentration above about 500 μM.

19. The method of claim 1, wherein the flavonoid is contacted/incubated with said glycosyl transferase at a final concentration above about 1 mM.

20. The method of claim 1, wherein contacting and/or incubating is/are done at a temperature from about 24° C. to about 30° C., preferably at a temperature of about 28° C.


SEQUENCE LISTING (79 sequences)

SEQ ID NO: 1 - protein, 376 aa, Artificial Sequence
  variable sequence of glycosyl transferase
  UNSURE 1..20: Xaa = any amino acid
  VARIANT 21: Lys = Arg
  UNSURE 26..27: Xaa = any amino acid
  UNSURE 29: Xaa = any amino acid
  VARIANT 34: Asn = Ser
  UNSURE 38: Xaa = any amino acid
  VARIANT 39: Leu = Ile
  UNSURE 41..46: Xaa = any amino acid
  UNSURE 48: Xaa = any amino acid
  VARIANT 53: Tyr = Phe
  UNSURE 54..84: Xaa = any amino acid
  VARIANT 85: Phe = Tyr or Leu
  VARIANT 87: Glu = Asp
  UNSURE 89..99: Xaa = any amino acid
  UNSURE 102: Xaa = Ala, Ile, Leu, Met, Phe, Pro, Trp or Val
  UNSURE 103..104: Xaa = any amino acid
  UNSURE 105: Xaa = Ala, Ile, Leu, Met, Phe, Pro, Trp or Val
  UNSURE 107..108: Xaa = any amino acid
  UNSURE 110..111: Xaa = any amino acid
  VARIANT 113: Tyr = Phe
  UNSURE 114: Xaa = Ala, Ile, Leu, Met, Phe, Pro, Trp or Val
  UNSURE 115: Xaa = any amino acid
  UNSURE 117..123: Xaa = any amino acid
  VARIANT 124: Phe = Trp
  UNSURE 127: Xaa = any amino acid
  UNSURE 128..130: Xaa = Ala, Ile, Leu, Met, Phe, Pro, Trp or Val
  UNSURE 131: Xaa = any amino acid
  VARIANT 132: Asp = Glu
  UNSURE 133..134: Xaa = any amino acid
  UNSURE 136..139: Xaa = any amino acid
  UNSURE 141..155: Xaa = any amino acid
  UNSURE 158: Xaa = any amino acid
  UNSURE 160: Xaa = Asn, Cys, Gln, Gly, Ser, Thr or Tyr
  UNSURE 161..163: Xaa = any amino acid
  VARIANT 165: Pro = Ala
  UNSURE 167: Xaa = any amino acid
  UNSURE 169: Xaa = any amino acid
  UNSURE 171..172: Xaa = any amino acid
  UNSURE 174..178: Xaa = any amino acid
  VARIANT 180: Lys = Arg
  UNSURE 181..229: Xaa = any amino acid
  UNSURE 232: Xaa = any amino acid
  VARIANT 233: Gly = Cys
  UNSURE 234: Xaa = any amino acid
  VARIANT 235: Pro = Lys
  VARIANT 238: Glu = Asp
  UNSURE 240: Xaa = any amino acid
  UNSURE 242..280: Xaa = any amino acid
  UNSURE 285: Xaa = Ala, Ile, Leu, Met, Phe, Pro, Trp or Val
  VARIANT 287: Lys = Arg
  UNSURE 288..290: Xaa = any amino acid
  UNSURE 292..294: Xaa = Ala, Ile, Leu, Met, Phe, Pro, Trp or Val
  VARIANT 301: Arg = Lys
  UNSURE 302..305: Xaa = any amino acid
  UNSURE 308..309: Xaa = Ala, Ile, Leu, Met, Phe, Pro, Trp or Val
  UNSURE 314..329: Xaa = any amino acid
  VARIANT 331: Glu = Asp
  UNSURE 337..338: Xaa = any amino acid
  VARIANT 339: Val = Ile
  UNSURE 342..343: Xaa = any amino acid
  VARIANT 346: Tyr = Phe
  VARIANT 347: Ile = Val
  VARIANT 348: Thr = Ser
  VARIANT 352: Tyr = Phe
  VARIANT 356: Met = Leu
  UNSURE 358: Xaa = any amino acid
  UNSURE 360: Xaa = any amino acid
  VARIANT 361: Asn = His
  UNSURE 362: Xaa = any amino acid
  UNSURE 365: Xaa = Ala, Ile, Leu, Met, Phe, Pro, Trp or Val
  UNSURE 367: Xaa = any amino acid
  UNSURE 370: Xaa = Ala, Ile, Leu, Met, Phe, Pro, Trp or Val
XXXXXXXXXXXXXXXXXXXXKILFAXXPXDGHFNPLTXLAXXXXXXGXDVRWYXXXXXXXXXXXXXXXXXXXXXXXXXXXXXXXFPERXXXXXXXXXXXFDXXXXFXXRXXEYXXDXXXXXXXFPFXXXXXDXXFXXXXFXXXXXXXXXXXXXXXPLXEXXXXLPPXGXGXXPXXXXXGKXXXXXXXXXXXXXXXXXXXXXXXXXXXXXXXXXXXXXXXXXXXXXXXXXLQXGXPGFEYXRXXXXXXXXXXXXXXXXXXXXXXXXXXXXXXXXXXXXXXXTQGTXEKXXXKXXXPTLEAFRXXXXLVXXTTGGXXXXXXXXXXXXXXXXIEDFIPFXXVMPXXDVYITNGGYGGVMLXIXNXLPXVXAGXHEGKNE

SEQ ID NO: 2 - DNA, 1380 bp, Artificial Sequence
  variable sequence of glycosyl transferase
  unsure 1..60: /replace=“a, t, g or c”
  variation 61..63: /replace=“mgr”
  unsure 75..81: /replace=“a, t, g, or c”
  unsure 84..87: /replace=“a, t, g or c”
  unsure 93: /replace=“a, t, g or c”
  variation 100..102: /note=“tcn - n can be any of t, c, g or a” /replace=“agy” /replace=“tcn”
  unsure 105: /replace=“a, t, g or c”
  unsure 111..114: /replace=“t, g, a or c”
  variation 115..117: /replace=“ath”
  unsure 120..138: /replace=“a, t, g or c”
  unsure 141: /replace=“a, t, g or c”
  unsure 150: /replace=“a, t, g or c”
  variation 157..159: /replace=“tty”
  unsure 160..207: /replace=“a, t, g or c”
  variation 208..210: /replace=“tay” /replace=“ytr”
  unsure 213: /replace=“a, t, g or c”
  variation 214..216: /replace=“gay”
  unsure 220..297: /replace=“a, t, g or c”
  unsure 304..315: /replace=“a, t, g or c”
  unsure 319..324: /replace=“a, t, g or c”
  unsure 329..333: /replace=“a, t, g or c”
  variation 337..339: /replace=“tty”
  unsure 340..345: /replace=“a, t, g or c”
  unsure 349..369: /replace=“a, t, g or c”
  variation 370..372: /replace=“tgg”
  unsure 375: /replace=“a, t, g or c”
  unsure 379..393: /replace=“a, t, g or c”
  variation 394..396: /replace=“gar”
  unsure 397..402: /replace=“a, t, g or c”
  unsure 406..417: /replace=“a, t, g or c”
  unsure 421..465: /replace=“a, t, g or c”
  unsure 468: /replace=“a, t, g or c”
  unsure 472..474: /replace=“a, t, g or c”
  unsure 481..489: /replace=“a, t, g or c”
  variation 493..495: /note=“n at position 495 = a, t, g or c” /replace=“gcn”
  unsure 498..501: /replace=“a, t, g or c”
  unsure 504..507: /replace=“a, t, g or c”
  unsure 510..516: /replace=“a, t, g or c”
  unsure 519..534: /replace=“a, t, g or c”
  unsure 537: /replace=“a, t, g or c”
  variation 538..540: /replace=“mgr”
  unsure 541..687: /replace=“a, t, g or c”
  unsure 694..696: /replace=“a, t, g or c”
  variation 697..699: /note=“n at position 699 = a, t, g or c” /replace=“tgy”
  unsure 700..702: /replace=“a, t, g or c”
  variation 703..705: /note=“n at position 705 = a, t, g or c” /replace=“aar”
  unsure 708: /replace=“a, t, g or c”
  variation 712..714: /replace=“gay”
  unsure 718..720: /replace=“a, t, g or c”
  unsure 724..840: /replace=“a, t, g or c”
  unsure 843: /replace=“a, t, g or c”
  unsure 849: /replace=“a, t, g or c”
  unsure 852..855: /replace=“a, t, g or c”
  unsure 862..870: /replace=“a, t, g or c”
  variation 871..873: /replace=“mgr”
  unsure 874..882: /replace=“a, t, g or c”
  unsure 885: /replace=“a, t, g or c”
  unsure 888: /replace=“a, t, g or c”
  unsure 897: /replace=“a, t, g or c”
  variation 901..903: /replace=“aar”
  unsure 904..915: /replace=“a, t, g or c”
  unsure 921..927: /replace=“a, t, g or c”
  unsure 930: /replace=“a, t, g or c”
  unsure 933: /replace=“a, t, g or c”
  unsure 936: /replace=“a, t, g or c”
  unsure 939..987: /replace=“a, t, g or c”
  variation 991..993: /replace=“gay”
  unsure 999: /replace=“a, t, g or c”
  unsure 1005: /replace=“a, t, g or c”
  unsure 1008..1014: /replace=“a, t, g or c”
  variation 1015..1017: /note=“n at position 1017 = a,t, g or c” /replace=“ath”
  unsure 1023..1029: /replace=“a, t, g or c”
  unsure 1035: /replace=“a, t, g or c”
  variation 1036..1038: /replace=“tty”
  variation 1039..1041: /note=“n on position 1041 = a, t, g or c” /replace=“gtn”
  variation 1042..1044: /note=“n on position 1043 = a, t, g or c” /replace=“tcn” /replace=“agy”
  unsure 1050: /replace=“a, t, g or c”
  unsure 1053: /replace=“a, t, g or c”
  variation 1054..1056: /replace=“tty”
  unsure 1059: /replace=“a, t, g or c”
  unsure 1062: /replace=“a, t, g or c”
  unsure 1065: /replace=“a, t, g or c”
  variation 1066..1067: /replace=“ytr”
  unsure 1072..1074: /replace=“a, t, g or c”
  unsure 1078..1080: /replace=“a, t, g or c”
  variation 1081..1083: /replace=“cay”
  unsure 1084..1086: /replace=“a, t, g or c”
  unsure 1092..1095: /replace=“a, t, g or c”
  unsure 1098..1101: /replace=“a, t, g or c”
  unsure 1104: /replace=“a, t, g or c”
  unsure 1107..1110: /replace=“a, t, g or c”
  unsure 1119: /replace=“a, t, g or c”
  unsure 1129..1380: /replace=“a, t, g or c”
nnnnnnnnnnnnnnnnnnnnnnnnnnnnnnnnnnnnnnnnnnnnnnnnnnnnnnnnnnnnaarathytrttygcnnnnnnnccnnnngayggncayttyaayccnytracnnnnytrgcnnnnnnnnnnnnnnnnnnnggntgtgaygtnmgrtggtaynnnnnnnnnnnnnnnnnnnnnnnnnnnnnnnnnnnnnnnnnnnnnnnnttyccngarmgrnnnnnnnnnnnnnnnnnnnnnnnnnnnnnnnnnnnnnnnnnnnnnnnnnnnnnnnnnnnnnnnnnnnnnnnnnnnnnnttygaynnnnnnnnnnnnttynnnnnnmgrnnnnnngartaynnnnnngaynnnnnnnnnnnnnnnnnnnnnttyccnttynnnnnnnnnnnnnnngaynnnnnnttynnnnnnnnnnnnttynnnnnnnnnnnnnnnnnnnnnnnnnnnnnnnnnnnnnnnnnnnnnccnytrnnngaragynnnnnnnnnytrccnccnnnnggnnnnggnnnnnnnccnnnnnnnnnnnnnnnnggnaarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnytrcarnnnggnnnnccnggnttygartaynnnmgrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacncarggnacnnnngaraarnnnnnnnnnaarnnnnnnnnnccnacnytrgargcnttymgrnnnnnnnnnnnnytrgtnnnnnnnacnacnggnggnnnnnnnnnnnnnnnnnnnnnnnnnnnnnnnnnnnnnnnnnnnnnnnnnathgargayttnathccnttnnnnnnngtnatgccnnnnnnngaygtntayathacnaayggnggntayggnggngtnatgytrnnnathnnnaaynnnytrccnnnngtnnnngcnggnnnncaygarggnaaraaygarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 3 - protein, 459 aa, Artificial Sequence
  glycosyltransferase GTC
MSNLFSSQTNLASVKPLKGRKILFANFPADGHFNPLTGLAVHLQWLGCDVRWYTSNKYADKLRRLNIPHFPFRKAMDIADLENMFPERDAIKGQVAKLKFDIINAFILRGPEYYVDLQEIHKSFPFDVMVADCAFTGIPFVTDKMDIPVVSVGVFPLTETSKDLPPAGLGITPSFSLPGKFKQSILRSVADLVLFRESNKVMRKMLTEHGIDHLYTNVFDLMVKKSTLLLQSGTPGFEYYRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 4 - DNA, 1380 bp, Artificial Sequence
  glycosyltransferase GTC
atgagtaatttattttcttcacaaacgaaccttgcatctgtaaaacccctgaaaggcaggaaaatactttttgccaacttcccggcagatgggcattttaatccattgacaggactggctgttcacttacaatggctgggttgtgatgtacgctggtacacttccaataaatatgcagacaaactgcgaagattgaatattccgcattttcctttcagaaaagctatggatatagctgacctggagaatatgtttccggagcgtgatgccattaaaggccaggtagccaaactgaagttcgacataatcaatgcttttattcttcgcgggccggaatactatgttgacctgcaggagatacataaaagttttccatttgacgtaatggtcgctgattgcgcttttacaggaattccttttgtaacagataaaatggatatacctgttgtttctgtaggtgtgttccctcttaccgaaacatcgaaagatcttcctcccgccggcctcgggattacgccttccttttctttacccggaaaatttaaacaaagcatactacggtcggtggctgacctggtcttattccgcgagtccaataaagtaatgagaaaaatgctgaccgaacatggcattgatcatctctatacaaatgtatttgacctgatggtaaaaaaatcaacgctgctattgcaaagcggaacaccgggttttgaatattaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 5 - protein, 440 aa, Artificial Sequence
  glycosyltransferase GTD
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGENVRFVGALLPYSESKSRQPWFDQKLLQYGRIVLVTQGTVEHDINKILVPTLEAFKNSETLVIATTGGNGTAELRARFPFENLIIEDFIPFDDVMPRADVYVTNGGYGGTLLSIHNQLPMVAAGVHEGKNEVCSRIGHFGCGINLETETPTPDQIRESVHKILSNDIFKKNVFRISTHLDVDANEKSAGHILDLLEERVVCG

SEQ ID NO: 6 - DNA, 1323 bp, Artificial Sequence
  glycosyltransferase GTD
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagcgacctgggcgaaaatgtccgttttgtcggcgcattgctgccgtactcggaaagtaaatcccggcagccctggtttgatcagaaacttttacaatatggcaggattgtgctggttacccagggcactgttgagcacgatatcaacaagatacttgtacccacgctggaagctttcaaaaattctgagacgctggtaattgccacaacaggcggtaatgggacagcggaattgcgcgcgcgttttcctttcgaaaacctgatcatcgaagatttcattccgtttgacgatgtgatgcccagagcagacgtttatgttaccaatggtggctatggaggcaccttgctcagcatacataatcagttgccaatggtagcggcgggcgtgcatgagggtaaaaatgaagtttgctcacgtatcggccacttcggctgtgggattaatctggaaacggaaacacctaccccagatcagatacgcgaaagtgtccacaaaatcctgtctaatgacatcttcaaaaagaatgtcttcaggatttcgacgcacttggatgtggatgcgaatgaaaaaagcgcgggtcacattcttgacttgttggaagagcgggttgtttgcggttaa

SEQ ID NO: 7 - protein, 441 aa, Artificial Sequence
  glycosyltransferase GTF
MTTKKILFATMPMDGHFNPLTGLAVHLHNQGHDVRWYVGGHYGAKVKKLGLIHYPYHKAQVINQENLDEVFPERQKIKGTVPRLRFDLNNVFLLRAPEFITDVTAIHKSFPFDLLICDTMFSAAPMLRHILNVPVAAVGIVPLSETSKELPPAGLGMEPATGFFGRLKQDFLRFMTTRILFKPCDDLYNEIRQRYNMEPARDFVFDSFIRTADLYLQSGVPGFEYKRSKMSANVRFVGPLLPYSSGIKPNFAHAAKLKQYKKVILATQGTVERDPEKILVPTLEAFKDTDHLVVITTGGSKTAELRARYPQKNVIIEDFIDFNLIMPHADVYVTNSGFGGVMLSIQHGLPMVAAGVHEGKNEIAARIGYFKLGMNLKTETPTPDQIRTSVETVLTDQTYRRNLARLRTEFAQYDPMALSERYINELLAKQPRKQHEAVEAI

SEQ ID NO: 8 - DNA, 1326 bp, Artificial Sequence
  glycosyltransferase GTF
atgacaactaaaaaaatcctgtttgccaccatgccaatggatggccacttcaaccccctgactggtctggctgttcatttgcataaccagggtcacgacgtacgctggtacgtgggcggacactacggtgccaaagtgaaaaagctgggcctgattcattacccttaccataaagcccaggttatcaatcaggagaatctggacgaggttttccctgaacgtcagaagatcaaagggaccgtaccccggctgcgctttgacctcaacaatgtcttcctgctgcgcgctcccgaattcattaccgacgttacggccatccacaaatcattcccattcgatctgctcatatgcgacaccatgttctcagcggctcccatgctgcgccatattctgaacgttccggtagcggccgtaggcattgtgcccctgagtgaaacctcgaaagaactgccaccggccggcctgggtatggagcctgctaccggtttctttgggcggctgaagcaggacttcctgcgctttatgactacccgtatcctcttcaagccctgcgacgatttgtacaacgagatccggcagcgctataacatggaaccagcccgtgattttgtcttcgactcgtttatccgcaccgccgatttgtacctgcaaagtggtgtaccgggctttgaatacaaacggagcaagatgagtgctaacgtccggtttgtcggcccgcttctcccctactccagcggtattaagccaaactttgcccatgcggccaaactgaagcagtataaaaaggtaattctggccacgcagggcacggtagaacgcgatccggagaagattctggtgccgacgctcgaagcgttcaaagacaccgatcacctggtcgtcataacaacgggcggttctaaaacggccgagttgcgcgcccggtatccgcagaaaaatgtcatcatcgaagacttcattgactttaacctcatcatgccccatgccgacgtatacgtaaccaattcgggtttcggcggagtgatgctgagcattcagcatggcctgccaatggtagctgccggtgttcacgagggtaaaaacgagattgcagcccgcattggctatttcaaactggggatgaatctgaagacagaaacccctacgccggaccagatccggacaagcgtcgaaacggttctgaccgatcagacctaccgccggaacttagcccggttgcggacggagttcgctcagtacgacccaatggcgttgagtgagcgatatatcaacgagctgctggccaaacaaccgcgcaagcaacacgaagccgtagaagcaatctaa

SEQ ID NO: 9 - protein, 454 aa, Segetibacter koreensis
  GT sequence
MKYISSIQPGTKILFANFPADGHFNPLTGLAVHLKNIGCDVRWYTSKTYAEKIARLDIPFYGLQRAVDVSAHAEINDVFPERKKYKGQVSKLKFDMINAFILRSTEYYEDILEIYEEFPFQLMIADITFGAIPFVEEKMNIPVISISVVPLPETSKDLAPSGLGITPSYSFFGKIKQSFLRFIADELLFAQPTKVMWGLLAQHGIDAGKANIFDILIQKSTLVLQSGTPGFEYKRSDLSSHVHFIGPLLPYTKKKERESWYNEKLSHYDKVILVTQGTIEKDIEKLIVPTLEAFKNSDCLVIATTGGAYTEELRKRYPEENIIIEDFIPFDDVMPYADVYVSNGGYGGVLLSIQHQLPMVVAGVHEGKNEINARVGYFDLGINLKTERPTVLQLRKSVDAVLQSDSYAKNVKRLGKEFKQYDPNEICEKYVAQLLENQISYKEKANSYQAEVLV

SEQ ID NO: 10 - DNA, 1365 bp, Segetibacter koreensis
  GT sequence
atgaaatatatttcatcgatacaaccgggaacaaaaatattatttgccaatttccctgccgatggtcacttcaatccgctgacaggattggctgttcatttaaaaaatattgggtgcgatgtgcgttggtacacttcaaagacatatgccgaaaaaattgccaggttagatatacctttttatggtttgcaaagagccgtagatgtaagtgcccatgcggaaatcaacgacgtttttcccgaaaggaaaaaatacaaaggccaggtaagcaagttgaaatttgatatgataaacgccttcattctgcgctctacggaatattatgaagacatattggaaatatacgaggaatttccttttcagttaatgattgctgacatcactttcggcgctattccttttgtagaagaaaaaatgaatattccggttatttccatcagcgttgttccgcttcccgaaacctcaaaagatctggctccctccggccttggtatcaccccttcttattcgttttttggcaaaataaaacagagctttttacgctttattgccgacgaattactttttgcgcaacccactaaagtaatgtggggccttttggcccaacatggaattgatgcggggaaagccaacatatttgacatacttatacaaaaatcaacactggtactacaaagcggcactccgggttttgaatacaagagaagtgacttaagcagtcatgtgcattttattggtccgctgctgccttacacaaaaaagaaagaaagagaaagctggtacaatgaaaagttaagccactacgataaagttattcttgtaacacaaggcacaattgaaaaagatattgagaagcttattgtgccaactcttgaagcatttaaaaactccgattgcctcgttattgctactactggcggtgcctatactgaagagttgagaaaacgttaccccgaggaaaatataattatagaagattttatcccttttgatgatgtaatgccttatgcagacgtatatgtttcaaacgggggatatggcggagttcttttatctatacaacatcaactgcctatggtagtggctggtgtacatgaaggaaaaaatgagattaatgcaagagtgggatattttgatttgggcattaatcttaagaccgaaagacctaccgtacttcaattaagaaaaagtgttgacgcagtcttacaaagtgattcatacgcgaagaatgtaaaacggcttggtaaagaattcaaacaatatgatccgaatgaaatatgtgaaaaatatgtagcgcaactgctggaaaatcaaatttcttataaagaaaaagcaaatagctaccaggccgaagttttggtttaa

SEQ ID NO: 11 - protein, 447 aa, Flavihumibacter solisilvae
  GT sequence
MNHKHSRKILMANVPADGHFNPLTGIAVHLKQQGYDVRWYGSDVYSKKAAKLGIPYFPFSKALEVNSENAEEVFPERKRINSKIGKLNFDLQNFFVRRAPEYYADLIDIHREFPFDLLIADCMFTAIPFVKELMQIPVLSIGIAPLLESSRDLAPYGLGLHPARSWAGKFRQAGLRWVADNILFRKSINVMYDLFEEYNIPHNGENFFDMGVRKASLFLQSGTPGFEYNRSDLSEHIRFIGALLPYAGERKEEPWFDSRLNKFDRVILVTQGTVERDVTKIIVPVLKAFRDSNYLVVATTGGNGTKLLREQYKADNIIIEDFIPFTDIMPYTDVYVTNGGYGGVMLGIENQLPLVVAGVHEGKNEINARIGYFRLGIDLRNERPTPEQMRNAIEKVIANGEYRRNVQALAREFKTYAPLELTERFVTELLLSRRHKLVPVNDDALIY

SEQ ID NO: 12 - DNA, 1344 bp, Flavihumibacter solisilvae
  GT sequence
atgaatcacaaacattccaggaagatcctgatggccaacgtgcctgcggatggccactttaatccgctgaccggcatcgcggttcacctgaagcagcagggctacgatgtacgctggtatggctcggatgtttacagcaaaaaagccgcaaaactgggtattccttattttcctttcagcaaggctcttgaagtaaacagcgaaaatgccgaagaggtctttccggaaagaaaacgcattaacagcaagattggcaagctgaattttgatctgcagaacttctttgttcgccgcgcaccggaatattatgctgacctgatcgacattcaccgcgagttcccttttgacctgctgatcgctgactgtatgtttactgccataccgtttgttaaggaactcatgcagattcctgtgctgtcgatcggaattgcgccactgctggaatcttcccgcgacctggcaccgtatggcctgggccttcatcctgcccgcagctgggccggcaagtttcgccaggcaggcttacgctgggttgcagacaatatccttttccgcaaatccatcaacgtcatgtatgacctttttgaagagtataatatcccgcacaacggggagaatttctttgacatgggtgtaagaaaagcttccctgttcctccagagcggaacaccgggatttgaatataaccgcagcgacctgagtgaacatatccgtttcatcggcgcattgcttccttacgccggagaaagaaaagaagagccctggttcgacagccgcctgaacaaatttgaccgggtgatcctggttacccagggaactgtggaacgtgatgtgacaaagatcattgtgccggtactgaaagccttccgtgacagtaactacctcgtggtagccactaccggcggcaatggaaccaaattgctgcgggagcaatacaaggcagataatatcatcatcgaggattttattcctttcactgatatcatgccctatacggatgtatacgttaccaatggtggttatggtggtgtaatgctggggatagaaaaccagcttccacttgttgttgcaggcgttcacgaagggaaaaatgagatcaatgcaagaataggctatttcaggcttggtatagacctgcgcaacgaaagaccgacaccggaacagatgcgcaatgccattgaaaaagtcattgcaaacggtgaatatcgcaggaatgtgcaggcactggcccgcgaattcaaaacctacgcaccgcttgaattaacggaaaggtttgtgacagaactgctgctcagcaggcgacataaactggttccggtaaacgacgatgcgcttatttactaa

SEQ ID NO: 13 - protein, 463 aa, Cesiribacter andamanensis
  GT sequence
METSQKGGTQSPKPFRRILFANCPADGHFNPLIPLAEFLKQQGHDVRWYSSRLYADKISRMGIPHYPFKKALEFDTHDWEGSFPERSKHKSQVGKLRFDLEHVFIRRGPEYFEDIRDLHQEFPFDVLVAEISFTGIAFIRHLMHKPVIAVGIFPNIASSRDLPPYGLGMRPASGFLGRKKQDLLRFLTDKLVFGKQNELNRQILRSWGIEAPGHLNLFDLQTQHASVVLQNGTPGFEYTRSDLSPNLVFAGPLLPLVKKVREDLPLQEKLRKYKNVILVTQGTAEQNTEKILAPTLEAFKDSTWLVVATTGGAGTEALRARYPQENFLIEDYIPFDQIMPNADVYVSNGGFGGVLQAISHQLPMVVAGVHEGKNEICARVGYFKLGLDLKTETPKPAQIRAAVEQVLQDPQYRHKVQALSAEFRQYNPQQLCEHWVQRLTGGRRAAAPAPQSAGGQLLSLTLN

SEQ ID NO: 14 - DNA, 1392 bp, Cesiribacter andamanensis
  GT sequence
atggaaacttcacaaaaaggcgggactcagtcacccaaaccattcagaagaattctttttgccaactgcccggccgacgggcactttaatccgctcattccactggcggaattcctcaagcagcaggggcatgatgtgcgctggtactcctcccgcctgtatgccgataagatttcgcgcatgggcattccccattatccttttaaaaaggcgcttgaatttgacacccacgactgggaagggagctttcccgagcgcagcaaacacaaaagccaggtaggcaagctgcgcttcgatctggagcatgtgttcattcgccgcggccctgagtactttgaagatattcgagacctccaccaggagtttccctttgatgtgctggtggccgagatcagctttaccggtattgcattcatccgccacctgatgcacaagccggtgattgcggtgggcatttttcccaacatcgcatcttcgcgcgacttgcctccctatgggctgggcatgcgtcctgctagcgggtttctgggtagaaaaaagcaagacctgctgcgctttcttaccgacaagctggtgtttggaaaacagaacgagctgaatcggcagattctccgcagctggggaattgaggcccccgggcaccttaacctgtttgacctgcagacgcagcatgcctcggtggttttgcagaacggaaccccgggttttgagtacacccgcagcgacctgagtcccaacctggtatttgcaggccccctgttgccgttggtgaaaaaagtgcgggaagatctacccctgcaggagaagctcaggaagtacaaaaacgtaattctggtaacccagggcactgccgagcaaaataccgaaaagattctggcgcccacactggaagcctttaaagacagcacctggctggtggtggcaaccacaggaggagcgggcaccgaggcgctgagggccaggtatccccaggagaatttcctgatcgaagattatattccttttgatcagatcatgcccaatgccgatgtatatgtatcgaacggaggctttggaggcgtcctgcaggccatttcacaccaactgcccatggtagtggcaggggtacatgagggtaaaaatgagatctgtgcccgggtgggctattttaagctggggctcgacctgaagacggaaacccccaaaccagcccagataagagcggcggtagagcaggtgctgcaagacccccagtaccgccacaaggtgcaggccctgagtgctgaattccggcaatacaatccacaacagctgtgcgagcactgggtgcagcgcctgacaggcggacgtagagcggctgcacccgcacctcagtcggctggcgggcagctactttccctgacgctgaactaa

SEQ ID NO: 15 - protein, 450 aa, Niabella aurantiaca
  GT sequence
MYTKTANTTNAAAPLHGGEKKKILFANIPADGHFNPLTGLAVRLKKAGHDVRWYTGASYAPRIEQLGIPFYLFNKAKEVTVHNIDEVFPERKTIRNHVKKVIFDICTYFIERGTEFYEDIKDINKSFDFDVLICDSAFTGMSFVKEKLNKHAVAIGILPLCASSKQLPPPIMGLTPAKTLAGKAVHSFLRFLTNKVLFKKPHALINEQYRRAGMLTNGKNLFDLQIDKATLFLQSCTPGFEYQRAHMSRHIHFIGPLLPSHSDAPAPFHFEDKLHQYAKVLLVTQGTFEGDVRKLIVPAIEAFKNSRHLVVVTTAGWHTHKLRQRYKAFANVVIEDFIPFSQIMPFADVFISNGGYGGVMQSISNKLPMVVAGIHEGKNEICARVGYFKTGINMRTEHPKPEKIKTAVNEILSNPLYRKSVERLSKEFSEYDPLALCEKFVNALPVLQKP

SEQ ID NO: 16 - DNA, 1353 bp, Niabella aurantiaca
  GT sequence
atgtacacaaaaacagcaaacacaaccaatgccgctgctcccttacacggcggtgaaaaaaagaaaatcttatttgccaacatccctgccgacgggcatttcaaccctctaacgggattagccgttcggctcaaaaaagcagggcatgatgtccgctggtacaccggcgccagctatgcaccccgtatcgaacagctgggcattcccttctatctttttaacaaggcaaaagaggtaaccgttcacaacattgacgaagtatttcccgaaaggaaaacgatccggaatcatgtaaagaaagtcatctttgatatctgcacgtattttatcgaacgcggaacagaattttatgaagacataaaggacatcaataaaagtttcgatttcgacgtgctgatctgcgacagcgcttttaccggtatgtcgttcgtaaaagaaaaactaaacaagcatgcagtagccatcggcatcctccctttatgtgcctcttcgaaacagctacccccgcccatcatgggacttacaccggccaaaaccctggcaggaaaagccgtgcattcgtttttgcgttttcttaccaataaagtattgtttaaaaagccccacgcgctgatcaacgaacaataccgccgtgcaggcatgctgaccaatggcaaaaacctgtttgatctgcagatcgataaggcaacactgtttttacaaagctgtaccccggggtttgaataccaacgcgcgcatatgagccggcatatccattttataggccctttactgccctcccatagtgatgcccctgccccattccattttgaagacaaactgcatcagtatgcaaaagtgctgctggtaacgcagggaacctttgaaggagatgtgcgcaagctgatcgtgcccgcaattgaagcctttaaaaacagccgccacctggtggtggtaacaacggccggatggcatacccataaactgcgccagcggtataaagcatttgccaatgttgttattgaagactttattccgttcagccagatcatgccttttgccgatgtattcatttcaaacggtggttacggcggtgtgatgcaaagcataagcaataagctgccaatggtagtggccggcatacacgaagggaaaaacgaaatatgtgcccgggtgggatattttaaaacaggcatcaatatgcgcacggaacatcccaaaccggaaaaaataaaaacagctgtgaacgagatcctgagcaaccccctttaccggaaaagcgtggaacggctttcgaaggaattttcggagtacgacccgttggccctttgtgaaaaattcgtcaacgctttacccgtccttcagaaaccatag

SEQ ID NO: 17 - protein, 441 aa, Spirosoma radiotolerans
  GT sequence
MITPQRILFATMPMDGHFSPLTGLAVHLSNLGHDVRWYVGGEYGEKVRKLKLHHYPFVNARTINQENLEREFPERAALKGSIARLRFDIKQVFLLRAPEFVEDMKDIYQTWPFTLVVHDVAFIGGSFIKQLLPVKTVAVGVVPLTESDDYLPPSGLGRQPMRGIAGRWIQHLMRYMVQQVMFKPINVLHNQLRQVYGLPPEPDSVFDSIVRSADVYLQSGVPSFEYPRKRISANVQFVGPLLPYAKGQKHPFIQAKKALQYKKVILVTQGTIERDVQKIIVPTLEAFKNEPTTLVIVTTGGSQTSELRARFPQENFIIDDFIDFNAVMPYASVYVTNGGYGGVMLALQHNLPIVVAGIHEGKNEIAARIDYCKVGIDLKTETPSPTRIRHAVETVLTNDMYRQNVRQMGQEFSQYQPTELAEQYINALLIQEKSSRLAVVA

SEQ ID NO: 18 - DNA, 1326 bp, Spirosoma radiotolerans
  GT sequence
atgatcacaccccaacgcattttgtttgctaccatgccaatggatggccattttagtcctctcaccggtcttgccgttcacttaagtaaccttggccacgatgtccgctggtatgtgggcggtgagtacggcgaaaaagtacggaagcttaagttgcaccattatccattcgtgaacgcccgaaccatcaatcaggaaaatctggagcgtgagtttccggaacgggccgcccttaagggttcgattgcccggctacggttcgatattaagcaggtgtttctgcttcgtgctccggaattcgttgaggatatgaaagatatctaccagacgtggccgttcactctggtagtacatgatgtagccttcattgggggctcgttcattaagcaactattgcccgttaaaaccgtggcggtaggcgtagtacccctcacggagtcggacgattacctgccgccgtctggtctgggcaggcaacccatgcgcggcatagctggccgctggattcagcatctgatgcgctacatggtgcagcaggttatgttcaaacccatcaatgtcctgcacaatcaacttcgacaggtctatggtctgccgcctgagccggactccgtgttcgattcgatcgtacgttctgccgatgtttatctccaaagtggcgtacccagctttgagtaccctcgcaaacggataagtgccaatgttcagtttgtggggccgctgctcccctacgccaaaggtcaaaagcacccgtttatacaggcaaaaaaagcgttgcagtacaaaaaagttattttagtaactcaggggacgatagagcgggatgtccaaaaaatcattgtaccaaccctggaagcttttaaaaatgagcctactacgctggtgatcgtcacaactggtggctcccaaacgagtgagttgcgtgcgcgttttccgcaggaaaatttcattattgatgactttatcgattttaatgcggttatgccctatgccagtgtgtatgtaacaaacgggggctatggcggggtaatgcttgcgctgcaacacaacctgccgattgtcgtcgcgggaattcacgagggtaaaaacgagattgcagcccgcattgattactgtaaggtaggcatagacctgaagactgagacgcccagccccacccgcattcgccatgccgtcgaaactgtattgaccaatgacatgtaccggcagaatgtccgtcaaatggggcaagagttcagtcagtatcaaccaactgaactggcggaacaatacatcaatgcgcttttaatacaagagaaaagctcccggctggccgttgtggcctag

SEQ ID NO: 19 - protein, 440 aa, Fibrella aestuarina
  GT sequence
MNPQRILFATMPFDGHFSPLTNLAVHLSQLGHDVRWFVGGHYGQKVTQLGLHHYPYVKTRTVNQENLDQLFPERATIKGAIARIRFDLGQIFLLRVPEQIDDLRAIYDEWPFDLIVQDLGFVGGTFLRELLPVKVVGVGVVPLTESDDWVPPTSLGMKPQSGRVGRLVSRLLNYLVQDVMLKPANDLHNELRAQYGLRPVPGFIFDATVRQADLYLQSGVPGFEFPRKRISPNVRFIGPMLPYSRANRQPFEQAIKTLAYKRVVLVTQGTVERNVEKIIVPTLEAYKKDPDTLVIVTTGGSGTLALRKRYPQANFIIEDFIDFNAVMPYVSVYVTNGGYGGVMLALQHKLPIVAAGVHEGKNEIAARIGYCQVGVDLRTETPTPDQIRRAVATILGDETYRRQVRRLSDEFGRYNPNQLAEQYINELLAQSVGEPVAALS

SEQ ID NO: 20 - DNA, 1323 bp, Fibrella aestuarina
  GT sequence
atgaatccccaacgcatcctcttcgccaccatgccattcgacgggcactttagccccctcaccaacctggccgttcaccttagccaactcgggcacgatgtgcgctggtttgtgggtgggcattacggccagaaagtaacgcagctgggcctgcaccattacccgtacgtgaaaacgcgcaccgtcaatcaggaaaatctggatcagctcttccccgaacgggccaccatcaaaggcgccattgcccgcatccgtttcgacctgggccagattttcctgcttcgtgtgcccgaacagatcgacgacctcagggcgatttacgacgaatggccgtttgacctcattgtgcaggatctgggctttgtggggggtacgttcctgcgcgagctgctgccggtgaaggtagtgggcgtgggcgtggtgccactcaccgaatccgacgactgggtgcccccgaccagcctgggcatgaaaccgcagtcgggccgggtgggccggctggtaagtcggctgctcaactacctggtgcaggacgttatgctgaagcccgccaatgacctgcacaacgagttaagggcgcagtacggccttcggccggtgccgggttttatctttgatgccaccgttcggcaggccgatctgtacctgcaaagcggcgtgccgggttttgaatttccccgtaagcgcatcagccccaacgtgcggttcatcgggcccatgctgccctacagccgggcaaacaggcagccgtttgagcaggccatcaaaacgctggcctataagcgggtggtgctcgtcacgcaggggaccgtcgagcggaacgtggagaagatcatcgtgcccacgctggaagcctacaaaaaagatcccgatacgctggtgattgtgaccaccggcggctcaggtacgttggcgttgcggaaacggtacccacaggccaattttatcatcgaagactttatcgatttcaacgccgtgatgccctacgtgagtgtgtacgtgaccaacggcgggtatggcggcgtgatgctggcgctgcaacacaagctcccgattgtggcggcgggcgtgcatgaaggcaaaaacgaaatcgccgcccggatcggctactgccaggtgggtgtcgacctgcgcaccgaaacgcccacccccgaccagattcgccgggcggtggccaccatcctgggcgacgaaacctaccggcgtcaggtacgtcggttgagcgacgagtttggccggtataaccctaatcaactggccgaacagtacatcaacgagctactggcccagtcggtgggggagcccgttgccgccctgtcgtga

SEQ ID NO: 21 - protein, 434 aa, Aquimarina macrocephali
  GT sequence
MTRMSQKKILFACIPADGHFNPMTAIAIHLKTKGYDVRWYTGEGYKNTLHRIGIPYLPFQNAQELKIEEIDKMYPDRKMLKGIAHIKFDIINLFINRMKGYYEDIAEIHQVFPFDILVCDNTFPGSIVKKKLNIPIASIGVVPLALSAPDLPLYGIGHQPATTFFGKRKQNFIKLMADKLIFDETKVVYNQLLRSLDLSEEENLTIFDIAPLQSDVFLQNGIPEIDYPRYSLPESIKYVGALQVQTNNNNNQKLKKDWSAILDTSKKIILVSQGTVEKNLDKLIIPSLEAFKDSDYIVLVATGYTDTKGLQKRYPQQHFYIEDFIAYDAVMPHIDVFIMNGGYGSALLSIKHGVPMITAGVNEGKNEICSRMDYSGVGIDLKTEKPRAVTIQNATERILGTDKYLDTIQKIQQRMNSYNTLDICEQHISRLISE

SEQ ID NO: 22 - DNA, 1305 bp, Aquimarina macrocephali
  GT sequence
atgacacgaatgtcccaaaaaaaaattcttttcgcttgtatacctgcagacggtcattttaatcctatgacagctatagctattcatctaaaaacaaaagggtatgatgtaagatggtatactggggagggctataaaaacacactacacagaatagggataccttatttaccgttccaaaatgcgcaggagcttaaaattgaggagatagataaaatgtatccagatcgaaaaatgctaaaaggaatcgcacatattaagttcgatattattaatctgtttattaatagaatgaaagggtactatgaagatatcgcagagatacatcaagtttttccgtttgatattttggtatgtgacaacacttttcccgggtctattgttaagaaaaaacttaatatcccaattgctagtataggagttgtgcctttagcactttctgcacctgatcttccattatacggcattggtcatcagcctgctacaacttttttcggtaagagaaaacagaactttataaaactaatggcagataaactcatttttgatgaaacaaaagtagtatataatcaattattacgctcattggatttatccgaagaagaaaatctaactatttttgatatagctccattacaatcggatgtttttttgcaaaacggaattcctgagatcgattatccaaggtatagtcttcccgaatccataaaatacgttggagcactacaagtacagaccaacaataacaacaatcaaaagttaaaaaaggactggagtgctattttagatacgtcaaaaaaaatcatattagtatctcagggaaccgtagaaaaaaatcttgacaagcttattattccttctttagaagcttttaaagactcagattacatagtactggtagctactggttataccgacactaaaggtttacaaaaacgataccctcagcagcatttttatatcgaagatttcatagcctatgatgctgtaatgccacatatagatgtctttatcatgaatggaggatatggcagtgctttactaagtattaaacacggtgtaccaatgattaccgctggggttaacgaaggtaaaaatgaaatctgttcccgaatggattattctggagtcggtattgatctaaaaacagaaaaaccacgagcagtcacaatacaaaatgcaactgaaagaatattaggtacagataaatatttagacactatacagaaaatacaacagcgtatgaattcttataacacattagatatctgcgaacaacatatctcccgtcttatttcagaataa

SEQ ID NO: 23 - protein, 452 aa, Artificial Sequence
  Chimera 1
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 24 - DNA, 1359 bp, Artificial Sequence
  Chimera 1
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 25 - protein, 447 aa, Artificial Sequence
  Chimera 2
MSNLFSSQTNLASVKPLKGRKILFANFPADGHFNPLTGLAVHLQWLGCDVRWYTSNKYADKLRRLNIPHFPFRKAMDIADLENMFPERDAIKGQVAKLKFDIINAFILRGPEYYVDLQEIHKSFPFDVMVADCAFTGIPFVTDKMDIPVVSVGVFPLTETSKDLPPAGLGITPSFSLPGKFKQSILRSVADLVLFRESNKVMRKMLTEHGIDHLYTNVFDLMVKKSTLLLQSGTPGFEYYRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFDDVMPRADVYVTNGGYGGTLLSIHNQLPMVAAGVHEGKNEVCSRIGHFGCGINLETETPTPDQIRESVHKILSNDIFKKNVFRISTHLDVDANEKSAGHILDLLEERVVCG

SEQ ID NO: 26 - DNA, 1344 bp, Artificial Sequence
  Chimera 2
atgagtaatttattttcttcacaaacgaaccttgcatctgtaaaacccctgaaaggcaggaaaatactttttgccaacttcccggcagatgggcattttaatccattgacaggactggctgttcacttacaatggctgggttgtgatgtacgctggtacacttccaataaatatgcagacaaactgcgaagattgaatattccgcattttcctttcagaaaagctatggatatagctgacctggagaatatgtttccggagcgtgatgccattaaaggccaggtagccaaactgaagttcgacataatcaatgcttttattcttcgcgggccggaatactatgttgacctgcaggagatacataaaagttttccatttgacgtaatggtcgctgattgcgcttttacaggaattccttttgtaacagataaaatggatatacctgttgtttctgtaggtgtgttccctcttaccgaaacatcgaaagatcttcctcccgccggcctcgggattacgccttccttttctttacccggaaaatttaaacaaagcatactacggtcggtggctgacctggtcttattccgcgagtccaataaagtaatgagaaaaatgctgaccgaacatggcattgatcatctctatacaaatgtatttgacctgatggtaaaaaaatcaacgctgctattgcaaagcggaacaccgggttttgaatattaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagatttcattccgtttgacgatgtgatgcccagagcagacgtttatgttaccaatggtggctatggaggcaccttgctcagcatacataatcagttgccaatggtagcggcgggcgtgcatgagggtaaaaatgaagtttgctcacgtatcggccacttcggctgtgggattaatctggaaacggaaacacctaccccagatcagatacgcgaaagtgtccacaaaatcctgtctaatgacatcttcaaaaagaatgtcttcaggatttcgacgcacttggatgtggatgcgaatgaaaaaagcgcgggtcacattcttgacttgttggaagagcgggttgtttgcggttaa

SEQ ID NO: 27 - DNA, 1380 bp, Artificial Sequence
  codon optimized GTC sequence
atgtcaaacctgttctcatctcaaacaaacctggcctcggtaaaaccgttaaaaggtcgtaaaatccttttcgcaaattttcccgctgatggacactttaatccgttaactgggttagcagtccatttacaatggcttggttgcgatgtgcgttggtacacttcaaataagtacgccgataagcttcgtcgccttaacatccctcacttcccttttcgtaaggccatggatattgctgacttagaaaacatgtttcctgagcgtgatgccatcaaaggacaggtcgcaaaactgaagttcgacattattaatgctttcattctgcgcggccctgagtactacgtcgacttacaagaaattcataaatcctttccctttgacgttatggtcgctgattgcgcgtttacgggaatcccgttcgtaactgacaaaatggatattcccgtcgtatcggtcggggtctttccactgaccgagacttctaaagatttgcctccggccggattgggtattactccctcgttttccttgccaggtaagttcaagcaatcgattttacgcagtgtggccgatttggtgttatttcgtgagagcaataaggtcatgcgcaaaatgttgactgagcatggtattgaccacctttacacaaacgtatttgatcttatggttaaaaaatcaacgttactgttgcagtcagggactccgggcttcgagtattaccgtagtgatcttggtaagaatattcgttttatcggaagcttgcttccctatcagagcaaaaaacagactactgcttggagtgatgagcgtctgaatcgctatgaaaaaatcgtcgtagtcactcagggaactgtagagaaaaacatcgaaaagattttggtgccaacccttgaggctttccgcgacactgacctgcttgtgatcgcgacgacgggaggttcaggaaccgctgaattgaaaaaacgttaccctcagggcaacttaatcattgaggacttcattccatttggtgacattatgccatacgctgatgtatatatcaccaatggtggttacggcggagttatgcttggcatcgaaaatcaactgccccttgtcgtagccgggatccacgaaggaaagaacgagatcaacgcacgtattgggtactttgagcttggaatcaatctgaaaacggagtggccgaagccagagcagatgaaaaaagcgattgacgaagttatcggtaataagaagtacaaagagaatatcacaaaactggcgaaggaattctcaaactaccatcctaacgaattgtgcgcccaatacatctctgaagtcttacagaagaccggccgcttgtacatttcgtccaagaaggaagaagaaaagatttactaa

SEQ ID NO: 28 - DNA, 1323 bp, Artificial Sequence
  Codon optimized GTD sequence
atgaccaaatacaaaaatgagttgaccggcaaacgtattttgtttggaaccgtgcctggagatggacatttcaaccccttaacaggcttagccaagtacctgcaagaactgggctgcgatgtacgctggtatgcatctgatgtatttaagtgcaaactggagaagctgagcatccctcactatgggttcaagaaggcttgggatgtaaatggagtaaatgttaatgaaattcttccggagcgtcaaaagctgaccgaccctgcggaaaagctgagtttcgaccttatccacatttttggaaatcgcgctcctgaatattacgaggacatcttggaaattcacgagagttttcctttcgacgtcttcatcgccgactcctgcttcagtgctattcccttagtttccaagcttatgtctattcctgtcgtggcagtaggggtgatcccgctggcagaagagagtgtggacttagcaccatacggaactggcctgccgccagctgcgacagaagagcagcgcgccatgtatttcggcatgaaggacgcacttgccaacgtggtgttcaaaacagccattgactcgttttccgccattttagatcgttatcaagtgcctcacgagaaagcgatcttatttgatactcttattcgtcaaagcgatttgtttttgcaaatcggagccaaagctttcgagtatgaccgcagcgatttgggggaaaacgtgcgtttcgttggagccctgctgccttattcggagagcaaaagtcgtcaaccctggttcgatcaaaagttgttacaatatgggcgcattgtcttggtcactcaggggacggtggaacatgatattaataagattctggttcctactttagaggcatttaaaaactcggaaaccctggtcatcgcgacaacaggaggaaatggtacagcagaattacgtgcgcgctttcccttcgaaaacttgatcattgaggatttcattccgttcgacgacgtgatgccccgcgcggatgtatatgtcaccaatggaggctatggtggcacgctgctttcaattcacaaccaacttccgatggttgcagccggggtccatgagggcaaaaatgaggtgtgttcccgtatcgggcactttggctgtgggatcaatctggagacggagacgccgacaccagatcagattcgtgaatcagttcataaaatcctgtcgaacgacattttcaagaaaaacgtttttcgtatttcaactcatttggacgtcgatgctaacgagaaaagcgccggtcatatcttggatctgttggaggagcgtgtcgtttgtgggtaa

SEQ ID NO: 29 - DNA, 1326 bp, Artificial Sequence
  Codon optimized GTF sequence
atgacgaccaagaagattcttttcgcaactatgcctatggacggtcatttcaatcctctgacagggcttgcggtgcacttgcataaccaaggtcatgatgtccgctggtacgtcggcggacattatggcgcaaaggttaaaaaattaggattaattcattatccctatcacaaggctcaagtcattaatcaagaaaatctggacgaagtcttcccggagcgtcaaaagatcaaaggcactgtaccacgtttacgtttcgatcttaataatgtgttcttgctgcgcgctcccgaatttattaccgatgtcactgcgattcacaaatcgtttccttttgacctgctgatctgtgataccatgttctcggcggctccaatgttacgccacattttgaatgtacccgtcgcagcggtgggtattgtgccattgtcagaaacttccaaggaactgccgccagcggggttggggatggagccggcgacaggattctttggacgtttgaagcaggatttcttacgtttcatgaccactcgtatcctttttaagccgtgcgacgatttatacaacgagatccgccagcgctacaacatggagcccgcccgcgattttgtctttgactccttcatccgtacggcggatctgtacctgcagtcaggcgtccctggatttgagtacaagcgctcaaagatgtcggcgaatgtgcgtttcgtcggacccttactgccctatagttcagggatcaagcctaattttgcccatgccgctaaattgaaacagtacaaaaaggtcatcttagccacccagggaacagtcgagcgtgaccctgagaaaatcttagtaccaactcttgaagctttcaaggacaccgatcatctggttgtgattacgaccggaggctcgaagacagcggagctgcgcgctcgttaccctcagaagaacgtgattatcgaggatttcattgactttaacttaatcatgcctcatgcagatgtttacgtcaccaactctggttttggtggtgtgatgctttccattcagcatggtttgccaatggtagctgcaggagttcacgaggggaagaacgaaattgctgctcgcattgggtatttcaaattagggatgaacttgaaaaccgaaacgccgacacccgaccagatccgtacaagtgtagagactgttttgacggaccaaacctatcgtcgcaacttagcgcgtttacgcacggaattcgctcaatacgacccaatggcactgtcagaacgctatattaacgagttgcttgcgaagcagccacgcaaacagcatgaggcagtagaagcgatttaa

SEQ ID NO: 30 - DNA, 1365 bp, Segetibacter koreensis
  Codon optimized GT sequence
atgaaatatatcagctccattcagcccggcacaaaaattttattcgcaaactttccggctgacggacacttcaatccattgacgggcttggcagtgcacttgaaaaatattggctgtgatgtccgttggtacaccagtaaaacctatgccgagaagatcgctcgcctggatatcccattttatggactgcagcgtgcagttgatgtatctgcccacgcagagattaatgacgtgtttcctgaacgcaagaagtacaagggacaagtttcaaaattgaagtttgatatgatcaatgcgtttattctgcgcagtacagagtattacgaggacattttagaaatttatgaggagtttcctttccagcttatgatcgccgacattaccttcggcgcgattcccttcgttgaagaaaaaatgaacattccagtaatctccatttcggttgtaccgttacctgaaacgtcgaaagatcttgccccgagtggcctgggaattacgccatcatactcgttctttggtaaaattaagcaatcgttcttacgtttcattgccgacgagcttttattcgcgcaacctaccaaggtcatgtggggtttattagcccaacacggaattgacgcggggaaagctaacatctttgacatcttgatccaaaagagtacgctggtattgcagagtggtacgccagggtttgagtacaaacgttctgatttgagctcccacgtgcatttcatcggcccgctgttaccctacactaagaagaaggaacgcgaatcatggtataatgaaaaattgtctcattatgataaggtcattttggtaacccaggggacaatcgaaaaagatattgagaaattaattgtaccgactttggaggcctttaagaattccgattgcctggtgattgcgacgacgggtggggcttacactgaagaattgcgcaaacgctatcctgaggaaaatattatcatcgaagacttcattccgtttgacgacgtaatgccgtatgccgacgtttacgtttcgaacggaggctatggtggcgtattgttatcaattcaacaccaactgcctatggtcgtcgcaggagttcatgagggtaaaaacgagatcaatgcgcgtgtaggttactttgaccttggcatcaatttgaagaccgagcgccccaccgttcttcaattgcgcaagagtgtagatgccgtgttacagtccgacagttatgcgaaaaacgtgaagcgtctgggaaaggagtttaagcaatacgatcctaatgaaatctgcgaaaagtacgtagcgcaactgcttgagaatcaaatcagctacaaggagaaggcgaattcctatcaggccgaagttctggtttaa

SEQ ID NO: 31 - DNA, 1344 bp, Flavihumibacter solisilvae
  Codon optimized GT sequence
atgaatcataagcattcgcgtaagatcctgatggcgaacgttcccgccgatggtcattttaatcccctgactggaattgcggtccaccttaagcagcaaggctacgatgtacgttggtatggatcagacgtttatagcaaaaaggcggccaaattagggattccgtatttcccattttcaaaagcgttggaagttaattcagaaaatgcagaagaagtgttccccgaacgtaagcgcatcaattcgaagattggaaaattaaatttcgatttgcagaatttctttgttcgtcgtgcgccagagtactatgccgatcttattgatattcaccgtgagtttcctttcgacttgttaattgcggattgtatgttcacagcgatcccttttgttaaggagttgatgcagatcccggtgctgtctattggcattgcgccattgcttgaatcatcgcgtgatttggctccgtacggattgggtctgcatccggctcgtagctgggcggggaaattccgtcaagcgggactgcgctgggttgctgataacatcctttttcgtaaatcaatcaatgttatgtacgacctgttcgaggaatataatattcctcacaatggagaaaactttttcgacatgggcgttcgtaaagcttcactgttcctgcaatcgggtacgccgggttttgagtacaatcgcagcgatttatctgagcatatccgcttcatcggagcacttcttccgtacgctggtgaacgcaaggaggaaccctggttcgacagtcgcctgaacaaattcgaccgtgtcattctggttacacaagggactgttgaacgtgacgttacaaaaatcatcgtaccagtgttgaaagccttccgtgattcgaattacttggttgtcgcgacgactgggggaaatggtacaaagcttcttcgtgagcagtataaggctgacaatatcattatcgaggacttcattccatttaccgatattatgccctatactgatgtttacgtaactaacgggggctacggtggagtgatgttaggaatcgaaaatcaattacctttagtagtggcaggtgtgcacgaggggaagaatgagatcaatgcccgcatcgggtatttccgcttaggcattgatctgcgtaatgaacgtcccacccccgaacaaatgcgtaacgcgattgaaaaagtaatcgcaaacggagaatatcgtcgcaacgtccaagcgcttgcacgtgagtttaaaacatacgctcccttggagttgaccgagcgtttcgtcacagaactgttgttgtcacgtcgccacaaattggtccccgtcaacgatgacgctttgatctactaa

SEQ ID NO: 32 - DNA, 1392 bp, Cesiribacter andamanensis
  Codon optimized GT sequence
atggagacgagtcaaaaaggaggaacgcagtcgccaaagcccttccgccgtatcttatttgcaaattgtcctgcggatgggcatttcaaccctttaattcctttggctgagtttttgaagcaacaaggtcatgacgtacgctggtatagctcgcgtttatatgcggataagatttcacgtatgggcatcccgcactacccattcaaaaaggcgctggaatttgacacccacgattgggaaggcagctttccagaacgtagcaagcataagtcgcaagtaggcaagttacgttttgatctggaacatgtcttcatccgtcgcgggcccgaatactttgaggatattcgcgatttacaccaggagtttcctttcgatgttttagtggcagaaatcagctttacggggatcgcatttatccgccatctgatgcacaagcccgtgatcgcagtcggcattttcccgaacattgcttcctcacgcgacttacctccatacggactgggcatgcgtccagcttctggatttttgggtcgtaagaaacaggacttactgcgttttttaaccgacaagttggtcttcggtaagcaaaatgagttaaaccgtcaaattcttcgctcatggggcatcgaggctcctggccacctgaatctttttgacctgcagacacagcacgcgtctgtagttcttcagaatggtacccctggatttgagtacacccgttccgatctgagcccaaacttggtatttgctgggcctctgctgcctcttgtcaaaaaggtgcgcgaagatttgccgttgcaggagaaattgcgcaaatataaaaacgtcatcctggtgacacaggggaccgctgaacagaacacagaaaagatcttagctcccacccttgaagcattcaaagactccacttggcttgtcgtggcgacaactggcggagcggggaccgaagctttacgcgctcgctatccacaagaaaatttcttaatcgaggactatattcccttcgatcagatcatgccaaacgcggatgtttatgtgtcgaatggggggttcgggggtgtgcttcaggcgatctcacatcagcttccgatggtggtggccggcgtacacgagggtaaaaatgagatttgcgcccgcgtgggttacttcaaattgggacttgatctgaagaccgagaccccgaagcctgctcaaattcgcgcagcggtagaacaagttcttcaagatccacagtaccgccataaggttcaggcgttgtcagccgaattccgccaatataacccgcagcaattatgcgaacattgggtgcaacgtttaacggggggccgccgtgccgccgcccccgccccgcagtccgccgggggccagttattgagtttgacccttaattaa

SEQ ID NO: 33 - DNA, 1353 bp, Niabella aurantiaca
  Codon optimized GT sequence
atgtatacaaaaaccgcgaacacgaccaacgcggcagcgccattacacggaggcgaaaagaaaaagattttgtttgcaaacatcccagcagatgggcacttcaacccgttgacgggactggctgtccgccttaaaaaagcgggccacgatgtgcgctggtatacgggggcgtcgtatgcaccccgcatcgagcaactggggattcctttttatttattcaacaaagccaaagaagttacagttcataatattgatgaagtattcccagaacgtaaaacgatccgcaatcacgtcaaaaaagtcatcttcgatatctgtacttactttatcgaacgtgggaccgaattctatgaagatattaaagatatcaacaagagcttcgacttcgatgttcttatttgcgatagtgcctttacgggaatgtcctttgtaaaagaaaaattaaataagcatgcagtcgcaattggcattttgcccctttgcgcttcgtctaaacagctgccccccccaattatggggttaactccggcgaagaccctggcaggaaaggctgtgcactcgttccttcgctttcttactaacaaggtattgtttaaaaagccgcatgccttaatcaacgagcagtatcgtcgcgcgggaatgctgacgaacggtaagaacttattcgatttgcagattgataaagctacattattcttgcaatcctgcaccccaggcttcgaataccaacgcgctcatatgtctcgccatatccatttcatcggcccattattgccgtcacactcggatgcgcctgcaccatttcactttgaagacaaacttcatcagtacgctaaggtactgttggtgactcaaggcacattcgaaggtgacgttcgcaagcttattgttcctgcaattgaagcgttcaaaaattcgcgccatttggtagtcgtcacaacggcgggctggcacacccataagctgcgtcagcgctataaagccttcgcgaatgttgttattgaagatttcattcccttctcccaaatcatgccatttgcagacgtcttcattagtaacggtggatatggtggtgtaatgcagtccatttcaaataaactgcctatggtggttgctgggattcatgagggtaaaaatgaaatctgcgctcgcgtgggttatttcaagaccggaattaacatgcgtaccgagcatccaaaaccggaaaagattaaaaccgcagtaaatgagattctttctaatccgttgtatcgcaaatcagtggaacgtctgagtaaagagttctccgaatatgaccccttagcgttatgcgaaaagttcgtcaacgctcttcccgtcttacagaagccctag

SEQ ID NO: 34 - DNA, 1326 bp, Spirosoma radiotolerans
  Codon optimized GT sequence
atgatcactccacagcgcattttgtttgcgacgatgccgatggacggtcatttttctcccctgacgggtcttgccgtgcacttatcgaatttagggcacgatgttcgctggtacgtgggcggagagtatggcgaaaaggtgcgcaagttgaagttgcaccattatccctttgtcaacgctcgcacaattaatcaagagaatcttgagcgtgaattccctgagcgcgccgcgttaaagggtagcattgcccgtcttcgttttgacatcaagcaggtttttctgttgcgtgcaccagaattcgtggaagatatgaaagatatttaccaaacctggccctttacacttgtggttcacgacgtcgcctttattggtggaagctttattaaacagttgttacccgtaaaaacagtagcggttggagtcgtgccacttactgaatcggatgattacttaccaccctccggtcttggccgccaaccgatgcgcggaatcgccggtcgctggatccaacatctgatgcgctacatggttcagcaagtcatgtttaagccaatcaacgtcctgcataaccaacttcgtcaggtctatggtctgcctccggaaccggacagtgtctttgacagtatcgtgcgctctgccgatgtgtacttgcagtccggcgtaccgtcttttgagtatccacgcaagcgtatctcagctaatgttcaatttgtgggccctctgcttccgtatgctaaaggacaaaaacacccctttattcaggccaaaaaagccttgcagtacaagaaggttattctggtaactcaaggtactattgaacgcgatgtgcaaaaaattatcgtcccgacgctggaggcatttaagaacgaaccaacaactttggtcatcgtaacaaccgggggttcccagactagcgagctgcgtgcgcgctttccacaagagaatttcattatcgacgacttcattgattttaatgcagtaatgccatacgcgagcgtttacgtcactaatgggggctatggtggtgttatgttagctctgcaacacaacttgccgattgttgtagcgggaatccatgaaggaaagaacgagattgctgcccgcattgattactgcaaggtcggtatcgacctgaagactgagacccctagtccgacacgcattcgtcacgcggtggagactgttttgaccaatgacatgtaccgtcaaaatgttcgccagatggggcaggaattttcgcagtaccaacctactgagttagctgaacaatacattaatgcactgctgatccaggagaaatcaagccgtttggcagttgtagcctag

SEQ ID NO: 35 - DNA, 1323 bp, Fibrella aestuarina
  Codon optimized GT sequence
atgaatccccagcgcattcttttcgccacgatgcccttcgacggacacttctctccacttactaatttggccgttcacctttcacagctgggacacgacgtccgttggttcgtgggcgggcactacggtcagaaagtaacgcagttagggttacaccactatccctacgtaaaaacccgcaccgttaaccaggagaatctggatcaattgttccctgagcgtgccacaattaaaggcgccattgcccgtattcgtttcgatttaggacaaatctttctgcttcgtgttcctgaacagatcgacgatttgcgtgcgatttatgacgaatggcccttcgatcttatcgtacaagacttggggttcgtcggtggcacatttttacgtgagcttttacccgtgaaagttgtgggggtgggcgtcgtaccgttaactgagtcggatgattgggtaccccctacttcattaggtatgaagccccaatccggtcgcgtgggacgtttagtgtcgcgtcttttaaattatcttgttcaggacgtgatgctgaagcccgctaacgacttacacaatgaattgcgcgcgcagtacggactgcgccccgtgcccggcttcatttttgatgcaactgttcgtcaggcagacttataccttcagagcggggtaccaggatttgaatttcctcgcaaacgcatttcaccgaacgtacgttttatcggacccatgttaccctattcccgcgctaatcgtcaaccatttgaacaggcgatcaaaacacttgcgtacaaacgcgtggtgttggtaactcaaggaacagtagagcgcaacgtcgagaagattatcgttccaacgcttgaggcgtataagaaagatccagataccttagtgatcgtaactaccggtggctcgggtacgcttgcattacgtaaacgttacccacaagctaattttatcattgaagactttattgactttaacgcagtaatgccctacgtcagcgtttacgtaaccaacggcggctatgggggagtcatgttggctttgcagcataaattgcctattgtggccgcgggagtgcatgaagggaagaatgagatcgctgcgcgtattgggtactgtcaggtgggcgtcgatcttcgtaccgagactccgactcccgatcaaattcgtcgtgccgttgctacaattctgggagatgagacttaccgccgccaagtccgtcgtctgagcgacgagttcggtcgctataacccaaaccaacttgcggagcagtatattaacgaattgcttgctcaatcggttggggaacccgttgccgcgctgagctga

SEQ ID NO: 36 - DNA, 1305 bp, Aquimarina macrocephali
  Codon optimized GT sequence
atgacgcgcatgagtcagaagaagattttattcgcttgcattcccgcagacggccattttaatccaatgacggctatcgcaatccatttaaaaaccaagggatacgacgtacgctggtataccggggaggggtataaaaacacgttgcaccgcattggcatcccctatcttcccttccaaaacgcgcaagagctgaagattgaggaaattgacaaaatgtacccggatcgtaagatgttgaagggcattgcacacattaagttcgacatcatcaatttgttcatcaaccgcatgaagggttactatgaggatatcgccgagattcaccaagtttttccatttgatattttggtgtgtgacaatacgttccccgggtccattgttaagaagaagttgaatatccccattgcgtcgatcggagtggtccccctggccttatcagcaccagacttaccgttatacggaattggtcatcagccggctactacgttcttcggaaagcgtaaacaaaattttatcaaacttatggcagacaagttgatcttcgacgaaactaaggttgtatataaccagctgcttcgttccttggatctgtcggaggaggaaaaccttacaatcttcgatattgcccccttacagtctgatgtattcttacagaacggcatccccgagatcgactacccccgctattccttaccagagtccattaagtacgtgggagcgctgcaagtccagactaataacaacaataatcaaaagctgaagaaggattggagcgcgattttggatacatcaaaaaagatcatcctggttagccagggaacagtagaaaaaaacctggacaaacttattatccccagtttagaagcgttcaaagacagcgattatattgtactggtggctacgggttacactgacacaaaaggtttgcaaaaacgttatccgcagcaacacttttatatcgaagatttcattgcctatgacgccgtcatgcctcatattgatgtctttatcatgaacggcggttatggatcggcactgttgagcattaagcatggtgtcccgatgattacggcaggcgtgaatgaggggaaaaacgaaatctgttcacgcatggattattcaggtgttggaatcgacctgaagacagaaaagcctcgtgccgttacaatccaaaacgccacagaacgcattttagggacggacaagtacctggacacgattcagaagattcagcaacgtatgaactcctacaatacattagacatttgcgagcagcacatctcgcgcctgatttcggagtaa

SEQ ID NO: 37 - DNA, 1359 bp, Artificial Sequence
  Codon optimized sequence of Chimera 1
atgaccaaatacaaaaatgagttgacaggcaaacgtattcttttcggtacagttcccggtgatggacactttaacccattaacaggcttagctaaatatttgcaggaattagggtgtgacgtgcgttggtatgcttcggatgtcttcaagtgcaagttagaaaaacttagtatccctcattatggatttaaaaaagcatgggacgttaatggcgtaaatgttaacgaaatcctgcctgaacgtcaaaaattgaccgatcccgctgaaaagttaagtttcgatctgatccatatttttggtaaccgcgcgcccgagtactacgaggacattcttgaaattcatgagagttttccctttgacgtctttattgctgatagttgcttttcggcaattcccttggtgtctaaattgatgagcattccagtagtagcggtcggggtgattcctttggccgaagagtctgtcgatcttgccccatacggtactggattaccgccggcagccacggaagagcaacgtgctatgtactttggcatgaaagatgcacttgcaaacgtcgtgttcaaaactgcaattgacagcttttccgccatcctggaccgctaccaggtgccccatgaaaaggcaatcctgttcgataccttgatccgtcagtccgatcttttccttcaaatcggtgctaaggcttttgaatacgatcgtagcgacttggggaagaatattcgctttattggtagcttacttccttatcagtcgaagaagcaaacgacagcctggagtgacgagcgtttgaaccgctacgagaaaatcgtggtcgtgacccagggaactgttgaaaagaatattgaaaaaatcttagtgccgacattggaggccttccgcgatacggatttgctggtaatcgctacaactggtgggtccggtactgctgagttaaagaaacgttaccctcaggggaacttaattatcgaagatttcatccccttcggagatatcatgccatatgcggatgtctacatcacgaatggagggtacggtggagttatgttgggcattgagaatcaactgccgttagtcgtagcggggatccacgaggggaagaacgaaattaacgcacgcattgggtacttcgagttgggaattaacttaaaaactgaatggcctaagcccgaacaaatgaaaaaggccatcgacgaagtaattggtaacaaaaaatataaggagaacatcacgaaacttgctaaggagttctcaaactaccacccaaacgaattatgcgcacagtacatctctgaagtattgcagaagaccggtcgtctgtacatctcgtcgaagaaggaggaagaaaagatctactaa

SEQ ID NO: 38 - DNA, 1344 bp, Artificial Sequence
  Codon optimized sequence of Chimera 2
atgtccaaccttttttcgtcccagacgaatcttgccagcgtaaaacctttaaaagggcgcaagattctttttgcaaattttcccgccgacgggcatttcaatcctcttacaggcttggccgtacacttacaatggcttgggtgtgacgtgcgttggtatacttcaaacaagtatgccgacaagctgcgtcgtttgaatatcccgcattttccttttcgcaaagccatggacattgctgaccttgagaacatgtttccagagcgcgacgccatcaaggggcaagtcgctaaattgaaattcgatatcattaacgcatttatcctgcgcggtccggagtattacgtagatttgcaggagattcacaagtcatttccattcgatgtcatggttgctgattgcgcctttacaggaattccattcgtcacagacaaaatggatatccccgtggtctcggtaggcgtatttcccttaaccgagaccagcaaagatcttccacccgcagggttggggatcactccatccttctcccttcctggaaagttcaagcaaagcattcttcgctcggttgccgacttagtcttattccgcgaatctaataaagttatgcgcaaaatgttgacggaacatggcattgaccatctttatactaatgtgttcgacttgatggtcaaaaaaagcaccctgttactgcaaagcgggacgccgggttttgaatattaccgcagcgatctgggcaagaatatccgctttatcggctcccttcttccgtatcaatctaagaaacagacaaccgcatggagcgatgagcgtctgaaccgctatgaaaagattgtcgttgtcacccaagggaccgtcgaaaaaaatattgagaaaatcttggttcctaccttagaggcatttcgtgacactgatcttttagtgatcgcaaccacaggtggtagcggaacagcagagttaaaaaagcgctacccccaaggaaatcttatcattgaagatttcattccgtttgacgacgttatgcctcgcgccgatgtatacgtgactaatggaggatacggaggtacgttactgtctatccataatcagctgccaatggtcgccgccggcgttcacgaaggcaagaatgaagtatgttcccgtattgggcattttggatgtggaatcaatttggaaaccgaaaccccaacccctgaccagattcgcgagtcagttcacaaaatcttgtcaaatgacatcttcaagaagaatgtattccgcattagcacacatttggatgtcgatgcgaacgagaaaagcgccgggcacattttagacttactggaagaacgcgtagtatgtggataa

SEQ ID NO: 39 - DNA, 29 bp, Artificial Sequence
  GTC-Ndel-for
catatgagtaatttattttcttcacaaac

SEQ ID NO: 40 - DNA, 27 bp, Artificial Sequence
  GTC-BamHI-rev
ggatccttagtatatcttttcttcttc

SEQ ID NO: 41 - DNA, 28 bp, Artificial Sequence
  GTF_XhoI_for
ctcgagatgacgaaatacaaaaatgaat

SEQ ID NO: 42 - DNA, 25 bp, Artificial Sequence
  GTF_BamHI_rev
ggatccttaaccgcaaacaacccgc

SEQ ID NO: 43 - DNA, 29 bp, Artificial Sequence
  GTL_XhoI_for
ctcgagatgacaactaaaaaaatcctgtt

SEQ ID NO: 44 - DNA, 26 bp, Artificial Sequence
  GTL_BamHI_rev
ggatccttagattgcttctacggctt

SEQ ID NO: 45 - protein, 20 aa, Artificial Sequence
  partial sequence of SEQ ID NO. 1
  VARIANT 1: Lys = Arg
  UNSURE 6..7: Xaa = any amino acid
  UNSURE 9: Xaa = any amino acid
  VARIANT 14: Asn = Ser
  UNSURE 18: Xaa = any amino acid
  VARIANT 19: Leu = Ile
KILFAXXPXDGHFNPLTXLA

SEQ ID NO: 46 - protein, 7 aa, Artificial Sequence
  partial sequence of SEQ ID NO. 1
  UNSURE 2: Xaa = any amino acid
  VARIANT 7: Tyr = Phe
GXDVRWY

SEQ ID NO: 47 - protein, 4 aa, Artificial Sequence
  partial sequence of SEQ ID NO 1
  VARIANT 1: Phe = Tyr or Leu
  VARIANT 3: Glu = Asp
FPER

SEQ ID NO: 48 - protein, 17 aa, Artificial Sequence
  partial sequence of SEQ ID NO 1
  UNSURE 3: Xaa = Ala, Ile, Leu, Met, Phe, Pro or Val
  UNSURE 4..5: Xaa = any amino acid
  UNSURE 6: Xaa = Ala, Ile, Leu, Met, Phe, Pro or Val
  UNSURE 8..9: Xaa = any amino acid
  UNSURE 11..12: Xaa = any amino acid
  VARIANT 14: Tyr = Phe
  UNSURE 15: Xaa = Ala, Ile, Leu, Met, Phe, Pro or Val
  UNSURE 16: Xaa = any amino acid
FDXXXXFXXRXXEYXXD

SEQ ID NO: 49 - protein, 17 aa, Artificial Sequence
  partial sequence of SEQ ID NO. 1
  VARIANT 1: Phe = Trp
  UNSURE 4: Xaa = any amino acid
  UNSURE 5..7: Xaa = Ala, Ile, Leu, Met, Phe, Pro or Val
  UNSURE 8: Xaa = any amino acid
  VARIANT 9: Asp = Glu
  UNSURE 10..11: Xaa = any amino acid
  UNSURE 13..16: Xaa = any amino acid
FPFXXXXXDXXFXXXXF

SEQ ID NO: 50 - protein, 25 aa, Artificial Sequence
  partial sequence of SEQ ID NO 1
  UNSURE 3: Xaa = any amino acid
  UNSURE 5: Xaa = Asn, Cys, Gln, Gly, Ser, Thr or Tyr
  UNSURE 6..8: Xaa = any amino acid
  VARIANT 10: Phe = Ala
  UNSURE 12: Xaa = any amino acid
  UNSURE 14: Xaa = any amino acid
  UNSURE 16..17: Xaa = any amino acid
  UNSURE 19..23: Xaa = any amino acid
  VARIANT 25: Lys = Arg
PLXEXXXXLPPXGXGXXPXXXXXGK

SEQ ID NO: 51 - protein, 12 aa, Artificial Sequence
  partial sequence of SEQ ID NO. 1
  UNSURE 3: Xaa = any amino acid
  UNSURE 5: Xaa = any amino acid
  UNSURE 6: Xaa = Phe or Lys
  UNSURE 11: Xaa = any amino acid
LQXGXXGFEYXR

SEQ ID NO: 52 - protein, 21 aa, Artificial Sequence
  partial sequence of SEQ ID NO 1
  UNSURE 5: Xaa = Ala, Ile, Leu, Met, Phe, Pro, Trp or Val
  VARIANT 7: Lys = Arg
  UNSURE 8..10: Xaa = any amino acid
  UNSURE 12..14: Xaa = Ala, Ile, Leu, met, Phe, Pro, Trp or Val
  VARIANT 21: Arg = Lys
TQGTXEKXXXKXXXPTLEAFR

SEQ ID NO: 53 - protein, 8 aa, Artificial Sequence
  partial sequence of SEQ ID NO 1
  UNSURE 3..4: Xaa = Ala, Ile, Leu, Met, Phe, Pro, Trp or Val
LVXXTTGG

SEQ ID NO: 54 - protein, 47 aa, Artificial Sequence
  partial sequence of SEQ ID NO 1
  VARIANT 2: Glu = Asp
  UNSURE 8..9: Xaa = any amino acid
  VARIANT 10: Val = Ile
  UNSURE 13..14: Xaa = any amino acid
  VARIANT 17: Tyr = Phe
  VARIANT 18: Ile = Val
  VARIANT 19: Thr = Ser
  VARIANT 23: Tyr = Phe
  VARIANT 27: Met = Leu
  UNSURE 29: Xaa = any amino acid
  UNSURE 31: Xaa = any amino acid
  VARIANT 32: Asn = His
  UNSURE 33: Xaa = any amino acid
  UNSURE 36: Xaa = Ala, Ile, Leu, Met, Phe, Pro, Trp or Val
  UNSURE 38: Xaa = any amino acid
  UNSURE 41: Xaa = Ala, Ile, Leu, Met, Phe, Pro, Trp or Val
IEDFIPFXXVMPXXDVYITNGGYGGVMLXIXNXLPXVXAGXHEGKNE

SEQ ID NO: 55 - protein, 6 aa, Artificial Sequence
  partial sequence of SEQ ID NO. 1
HEGKNE

SEQ ID NO: 56 - protein, 464 aa, Artificial Sequence
  Chimera 1 frameshift
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGKNIRFIGSLLPYQSKKQTTAWSDERLNRYEKIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKQAGFISAVKRKKKRYTKDPAANKARKEA

SEQ ID NO: 57 - DNA, 1395 bp, Artificial Sequence
  Chimera 1 frameshift
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagtgatctgggaaaaaatatccgtttcattggttcattattaccctaccagtcaaaaaaacaaacaactgcatggtctgatgaaagactgaacaggtatgaaaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaacaggcaggctttatatcagcagtaaaaaggaagaagaaaagatatactaaggatccggctgctaacaaagcccgaaaggaagcgtag

SEQ ID NO: 58 - protein, 452 aa, Artificial Sequence
  Chimera 3
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGENVRFVGALLPYSESKSRQPWFDQKLLQYGRIVLVTQGTVEHDINKILVPTLEAFKNSETLVIATTGGNGTAELRARFPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 59 - DNA, 1359 bp, Artificial Sequence
  Chimera 3
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagcgacctgggcgaaaatgtccgttttgtcggcgcattgctgccgtactcggaaagtaaatcccggcagccctggtttgatcagaaacttttacaatatggcaggattgtgctggttacccagggcactgttgagcacgatatcaacaagatacttgtacccacgctggaagctttcaaaaattctgagacgctggtaattgccacaacaggcggtaatgggacagcggaattgcgcgcgcgttttcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 60 - DNA, 1359 bp, Artificial Sequence
  Codon-optimized nucleotide sequence of Chimera 3 (optimized for E. coli)
atgaccaaatacaaaaatgagttgaccggcaaacgtattttgtttggaaccgtgcctggagatggacatttcaaccccttaacaggcttagccaagtacctgcaagaactgggctgcgatgtacgctggtatgcatctgatgtatttaagtgcaaactggagaagctgagcatccctcactatgggttcaagaaggcttgggatgtaaatggagtaaatgttaatgaaattcttccggagcgtcaaaagctgaccgaccctgcggaaaagctgagtttcgaccttatccacatttttggaaatcgcgctcctgaatattacgaggacatcttggaaattcacgagagttttcctttcgacgtcttcatcgccgactcctgcttcagtgctattcccttagtttccaagcttatgtctattcctgtcgtggcagtaggggtgatcccgctggcagaagagagtgtggacttagcaccatacggaactggcctgccgccagctgcgacagaagagcagcgcgccatgtatttcggcatgaaggacgcacttgccaacgtggtgttcaaaacagccattgactcgttttccgccattttagatcgttatcaagtgcctcacgagaaagcgatcttatttgatactcttattcgtcaaagcgatttgtttttgcaaatcggagccaaagctttcgagtatgaccgcagcgatttgggggaaaacgtgcgtttcgttggagccctgctgccttattcggagagcaaaagtcgtcaaccctggttcgatcaaaagttgttacaatatgggcgcattgtcttggtcactcaggggacggtggaacatgatattaataagattctggttcctactttagaggcatttaaaaactcggaaaccctggtcatcgcgacaacaggaggaaatggtacagcagaattacgtgcgcgctttcctcagggcaacttaatcattgaggacttcattccatttggtgacattatgccatacgctgatgtatatatcaccaatggtggttacggcggagttatgcttggcatcgaaaatcaactgccccttgtcgtagccggcatccacgaaggaaagaacgagatcaacgcacgtattgggtactttgagcttggaatcaatctgaaaacggagtggccgaagccagagcagatgaaaaaagcgattgacgaagttatcggtaataagaagtacaaagagaatatcacaaaactggcgaaggaattctcaaactaccatcctaacgaattgtgcgcccaatacatctctgaagtcttacagaagaccggccgcttgtacatttcgtccaagaaggaagaagaaaagatttactaa

SEQ ID NO: 61 - protein, 452 aa, Artificial Sequence
  Chimera 4
MTKYKNELTGKRILFGTVPGDGHFNPLTGLAKYLQELGCDVRWYASDVFKCKLEKLSIPHYGFKKAWDVNGVNVNEILPERQKLTDPAEKLSFDLIHIFGNRAPEYYEDILEIHESFPFDVFIADSCFSAIPLVSKLMSIPVVAVGVIPLAEESVDLAPYGTGLPPAATEEQRAMYFGMKDALANVVFKTAIDSFSAILDRYQVPHEKAILFDTLIRQSDLFLQIGAKAFEYDRSDLGENVRFVGALLPYSESKSRQPWFDQKLLQYGQIVVVTQGTVEKNIEKILVPTLEAFRDTDLLVIATTGGSGTAELKKRYPQGNLIIEDFIPFGDIMPYADVYITNGGYGGVMLGIENQLPLVVAGIHEGKNEINARIGYFELGINLKTEWPKPEQMKKAIDEVIGNKKYKENITKLAKEFSNYHPNELCAQYISEVLQKTGRLYISSKKEEEKIY

SEQ ID NO: 62 - DNA, 1359 bp, Artificial Sequence
  Chimera 4
atgacgaaatacaaaaatgaattaacaggtaaaagaatactctttggtaccgttcccggagacggtcattttaatccccttaccgggcttgctaaatatttacaggaattagggtgcgatgtcaggtggtatgcttctgatgttttcaaatgcaagcttgaaaaattgtcgataccacattatggcttcaaaaaagcatgggatgtcaacggtgtgaatgtaaacgagatcctgccggagcgacaaaaattaacagatcccgccgaaaaactgagctttgacttgatccacattttcggaaaccgggcacctgagtattatgaggatattctcgaaatacacgaatcgttcccattcgatgtgttcattgctgacagctgcttttccgcgattccgttagttagcaagctgatgagcatccccgttgttgccgttggcgtaattcctctggcggaagaatctgttgatctggcgccttatggaacaggattgccgcctgccgcgacggaggagcaacgtgcgatgtattttggtatgaaagatgctttggccaacgttgttttcaaaactgccattgactctttttcggccattctggaccggtaccaggtaccgcacgaaaaagcaattttattcgatacattgatccgtcaatccgacttgtttctgcaaattggcgcaaaagcatttgagtatgaccgcagcgacctgggcgaaaatgtccgttttgtcggcgcattgctgccgtactcggaaagtaaatcccggcagccctggtttgatcagaaacttttacaatatggcaaaattgtggtggtgacacagggcactgttgaaaagaatattgaaaagatcctcgtgcccactctggaagcctttagggatacagacttattggtaatagccacaacgggtggaagtggtacagctgagttgaaaaaaagatatcctcaaggcaacctgatcatcgaagattttattccctttggcgatatcatgccttatgcggatgtatatattaccaatggaggatatggtggtgtaatgctgggtatcgaaaaccaattgccattggtagtagcgggtattcatgaagggaaaaatgagatcaatgcaaggataggatactttgaactgggaattaacctgaaaaccgaatggcctaaaccggaacagatgaaaaaagccatagatgaagtgatcggcaacaaaaaatataaagagaatataacaaaattggcaaaagaattcagcaattaccatcccaatgaactatgcgctcagtatataagcgaagtattacaaaaaacaggcaggctttatatcagcagtaaaaaggaagaagaaaagatatactaa

SEQ ID NO: 63 - DNA, 1362 bp, Artificial Sequence
  Codon-optimized nucleotide sequence of chimera 4 (optimized for E. coli)
atgaccaaatacaaaaatgagttgaccggcaaacgtattttgtttggaaccgtgcctggagatggacatttcaaccccttaacaggcttagccaagtacctgcaagaactgggctgcgatgtacgctggtatgcatctgatgtatttaagtgcaaactggagaagctgagcatccctcactatgggttcaagaaggcttgggatgtaaatggagtaaatgttaatgaaattcttccggagcgtcaaaagctgaccgaccctgcggaaaagctgagtttcgaccttatccacatttttggaaatcgcgctcctgaatattacgaggacatcttggaaattcacgagagttttcctttcgacgtcttcatcgccgactcctgcttcagtgctattcccttagtttccaagcttatgtctattcctgtcgtggcagtaggggtgatcccgctggcagaagagagtgtggacttagcaccatacggaactggcctgccgccagctgcgacagaagagcagcgcgccatgtatttcggcatgaaggacgcacttgccaacgtggtgttcaaaacagccattgactcgttttccgccattttagatcgttatcaagtgcctcacgagaaagcgatcttatttgatactcttattcgtcaaagcgatttgtttttgcaaatcggagccaaagctttcgagtatgaccgcagcgatttgggggaaaacgtgcgtttcgttggagccctgctgccttattcggagagcaaaagtcgtcaaccctggttcgatcaaaagttgttacaatatgggcgcaaaatcgtcgtagtcactcagggaactgtagagaaaaacatcgaaaagattttggtgccaacccttgaggctttccgcgacactgacctgcttgtgatcgcgacgacgggaggttcaggaaccgctgaattgaaaaaacgttaccctcagggcaacttaatcattgaggacttcattccatttggtgacattatgccatacgctgatgtatatatcaccaatggtggttacggcggagttatgcttggcatcgaaaatcaactgccccttgtcgtagccggcatccacgaaggaaagaacgagatcaacgcacgtattgggtactttgagcttggaatcaatctgaaaacggagtggccgaagccagagcagatgaaaaaagcgattgacgaagttatcggtaataagaagtacaaagagaatatcacaaaactggcgaaggaattctcaaactaccatcctaacgaattgtgcgcccaatacatctctgaagtcttacagaagaccggccgcttgtacatttcgtccaagaaggaagaagaaaagatttactaa

SEQ ID NO: 64 - DNA, 38 bp, Artificial Sequence
  GTSopt_pET_fw
gggaattccatatgatgaaatatatcagctccattcag

SEQ ID NO: 65 - DNA, 33 bp, Artificial Sequence
  GTSopt_pET_rv
cgggatccttaaaccagaacttcggcctgatag

SEQ ID NO: 66 - DNA, 59 bp, Artificial Sequence
  Bridge_P1_pETGTD
gcggccatatcgacgacgacgacaagcatatgacgaaatacaaaaatgaattaacaggt

SEQ ID NO: 67 - DNA, 51 bp, Artificial Sequence
  Bridge_P1_pETGTD
ggaagaagaaaagatatactaaggatccggctgctaacaaagcccgaaagg

SEQ ID NO: 68 - DNA, 26 bp, Artificial Sequence
  Chim_P1_D_Nde_for
catatgacgaaatacaaaaatgaatt

SEQ ID NO: 69 - DNA, 20 bp, Artificial Sequence
  Chim_P1_D_rev
gcggtcatactcaaatgatt

SEQ ID NO: 70 - DNA, 21 bp, Artificial Sequence
  Chim_P1_C_for
agtgatctgggaaaaaatatc

SEQ ID NO: 71 - DNA, 29 bp, Artificial Sequence
  Chim_P1_C_Bam_rev
ggatccttagtatatcttttcttcttcct

SEQ ID NO: 72 - DNA, 33 bp, Artificial Sequence
  GTDopt_pEt_fw
gggaattccatatgatgaccaaatacaaaaatg

SEQ ID NO: 73 - DNA, 33 bp, Artificial Sequence
  Chim3_pET_rv
cgggatccttagtaaatcttttcttcttccttc

SEQ ID NO: 74 - DNA, 28 bp, Artificial Sequence
  1r-Chim3-opt-o(Chim3-opt)
tgccctgaggaaagcgcgcacgtaattc

SEQ ID NO: 75 - DNA, 28 bp, Artificial Sequence
  2f-Chim3-opt-o(Chim3-opt)
tgcgcgctttcctcagggcaacttaatc

SEQ ID NO: 76 - DNA, 40 bp, Artificial Sequence
  1f-Assembly-o(Vec)
tgacgataaggatcgatggggatccatgaccaaatacaaa

SEQ ID NO: 77 - DNA, 43 bp, Artificial Sequence
  1r-Assembly-o(Vec)
tatggtaccagctgcagatctcgagttagtaaatcttttcttc

SEQ ID NO: 78 - DNA, 32 bp, Artificial Sequence
  1r-Chim4_GTD-o(Chim4_GTC)
cgattttgcgcccatattgtaacaacttttga

SEQ ID NO: 79 - DNA, 28 bp, Artificial Sequence
  2f-Chim4_GTC-o(Chim4_GTD)
acaatatgggcgcaaaatcgtcgtagtc